(19) United States
(12) Patent Application Publication
(10) Pub. No.: US 2004/0229233 A1
(43) Pub. Date: Nov. 18, 2004

(54) Human housekeeping genes and human-tissue specific genes

(75) Inventors: Hiroyuki Aburatani, Tokyo (JP); Shogo Yamamoto, Tokyo (JP)

Correspondence Address: OLIFF & BERRIDGE, PLC, P.O. BOX 19928, ALEXANDRIA, VA, 22320, US

(73) Assignee: NGK INSULATORS, LTD., Nagoya-shi (JP)

(21) Appl. No.: 10/684,422

(22) Filed: Oct. 15, 2003

Related U.S. Application Data
(60) Provisional application No. 60/418,614, filed on Oct. 16, 2002.

(51) Int. Cl. C12Q 1/68; C07H 21/04; C12N 9/00

(52) U.S. Cl. 435/6; 435/69.1; 435/320.1; 435/325; 435/183; 536/23.2

(57) ABSTRACT

A human housekeeping gene which is a gene expressed commonly over 35 different human tissues selected from the group consisting of genes Nos.1 to 1189 on FIGS. 1 to 39, and a set of housekeeping genes consisting of two or more of the genes selected from said group.

### TECHNICAL FIELD

The present invention relates to a gene, which is expressed commonly over multiple different human tissues (housekeeping gene) and a tissue-specific gene, which is expressed specifically in each of the multiple different human tissues (tissue-specific gene).

### BACKGROUND ART

In a medical biological research field or an industrial field utilizing the result thereof, the significance of a DNA microarray is becoming greater (for a DNA microarray, see for example U.S. Pat. No. 5,474,796, Schena, M. et al., Proc. Natl. Acad. Sci. 93:10614-10619, 1996; PCT WO95/251116; PCT WO95/35505; Heller, R. A. et al., Proc. Natl. Acad. Sci. 94:2150-2155, 1997; U.S. Pat. No. 5,605,662 and the like).

For example, a DNA microarray capable of detecting an overall expression profile is utilized for identifying a pathogenic gene for the purpose of diagnosing or treating various diseases.

A research of a cancer subtype expression fingerprint employing a DNA microarray and the response thereof to a therapeutic option (see for example Golub et al., Science 286:531-537, 1999; Van'T Veer et al., Nature 415:530-536, 2002; Stauston et al., Proc. Natl. Acad. Sci. USA 98:10787-10792, 2001; Shipp et al., Nature Medicine 8:68-74, 2002; Yeoh et al., Cancer Cell 1:133-143, 2002) is also conducted extensively. For instance, the document of PCT WO99/50456 discloses a set of oligonucleotide probes, in which the respective probes hybridize with multiple genes regulated by p53 gene, and provides a cancer diagnosis using a DNA microarray fitted with this probe set.

Any of these prior art findings mainly focuses on a disease sample in a certain tissue. However, an understanding how agene is expressed in a normal tissue is very important to study the basic molecular biology similarly to the findings of pathogenic genes (see for example Warrington et al., Physiol Genomics 2:143-147, 2000; Butte et al., Physiol Genomics 7:95-96, 2001; Vaculescu et al., Nat Genet 23:387-388, 1999). In addition, such a normal human tissue expression database is very useful also as reference data in using a disease-associated gene in a diagnosis or therapy.

With this regard, Hsiao et al. reported 451 genes, which are expressed commonly in 19 normal human tissues as housekeeping genes (Hsiao et al., Physiol Genomics 7:97-104, 2001).

However, the human tissues are not limited to these 19 types, and it is also known that the gene expression profile varies depending also on the development stage. Accordingly, a further large number of the tissues should be researched for the purpose of identifying human housekeeping genes more accurately.

### DISCLOSURE OF INVENTION

The inventors analyzed as a large number of human tissues as possible for the expression genes, and finally became successful in identifying 1189 housekeeping gene populations expressed commonly over 35 different human tissues, and further listed these genes up sequentially in an order from high similarity of expression frequency in each tissue.

Further more, in each of the 35 human tissues, the inventorse also identified a population of tissue-specific genes, which are not expressed in other tissues.

The invention is based on a novel gene population as described above. That is to say, the invention provides a human housekeeping gene expressed commonly over 35 different human tissues, which gene is selected from the group consisting of genes Nos.1 to 1189 on FIGS. 1 to 39. One of the preferred embodiments of the housekeeping genes is a gene selected from genes Nos.1 to 200 on FIGS. 1 to 7, and the base sequences of the cDNAs that are transcription products of each gene, are any of SEQ ID Nos.1 to 200.

The invention also provides a transcription product of a housekeeping gene described above. It is preferred that the transcription product is an RNA or cDNA, and that a concrete example of the cDNA is a DNA fragment having any of the base sequences of SEQ ID Nos.1 to 200.

The invention also provides an oligonucleotide probe which hybridizes with the above-mentioned housekeeping gene or the above-mentioned transcription product (RNA or cDNA). In this case, the cDNA preferably has the base sequence of any of SEQ ID Nos.1 to 200.

The invention also provides a set of at least two housekeeping genes expressed commonly over 35 different human tissues, wherein each gene the set is selected from the group consisting of genes Nos.1 to 1189 on FIGS. 1 to 39. In this set of gene, each gene is preferably selected predominantly from the gene No. 1 on FIGS. 1 to 39. An example of the preferred embodiments of this gene set is a set of genes selected from genes Nos.1 to 200 on FIGS. 1 to 7, and the base sequences of the cDNAs which are the transcription products of each gene is SEQ ID Nos.1 to 200.

The invention also provides a set of transcription products of each gene of the set of genes described above. This set of transcription products are preferably a set of RNAs or cDNAs, and a typical example of the cDNAs is DNA fragments having the base sequence of any of SEQ ID Nos.1 to 200.

The invention also provides a set of oligonucleotide probes, each of which hybridizes with each gene of the set of genes described above, or with each RNA or cDNA of the set of transcription products described above. In this case, the cDNA is preferably one having the base sequence of any of SEQ ID Nos.1 to 200.

The invention also provides a DNA microarray carrying the set of transcription products or the set of probes described above.

The invention also provides the following tissue-specific genes and/or sets of genes each consisting of two or more of them.

A human whole brain-specific gene selected from the group consisting of genes Nos.1 to 294 on FIGS. 41 to 48, or a set of genes consisting of two or more of human whole brain-specific genes selected from said group.

A human amygdaloid body-specific gene, which is the gene No.295 on FIG. 48.

A human caudate nucleus-specific gene selected from the group consisting of genes Nos.296 to 303 on FIG. 48, or a set of genes consisting of two or more of human caudate nucleus-specific genes selected from said group.

A human callosum-specific gene selected from the group consisting of genes Nos.304 to 305 shown on FIGS. 48 to 49, or a set of genes consisting of two or more of human callosum-specific genes selected from said group.

A human hippocampus-specific gene, which is the gene No.306 on FIG. 49.

A human cerebellum-specific gene selected from the group consisting of genes Nos.307 to 353 on FIGS. 49 to 50, or a set of genes consisting of two or more of human cerebellum-specific genes selected from said group.

A human thalamus-specific gene selected from the group consisting of genes Nos.354 to 358 on FIG. 50, or a set of genes consisting of two or more of human thalamus-specific genes selected from said group.

A human pituitary gland-specific gene selected from the group consisting of genes Nos.359 to 383 on FIGS. 50 to 51, or a set of genes consisting of two or more of human pituitary gland-specific genes selected from said group.

A human spinal cord-specific gene selected from the group consisting of genes Nos.384 to 387 on FIG. 51, or a set of genes consisting of two or more of human spinal cord-specific genes selected from said group.

A human salivary gland-specific gene selected from the group consisting of genes Nos.388 to 401 on FIG. 51, or a set of genes consisting of two or more of human salivary gland-specific genes selected from said group.

A human thymus-specific gene selected from the group consisting of genes Nos.402 to 437 on FIGS. 51 to 52, or a set of genes consisting of two or more of human thymus-specific genes selected from said group.

A human thyroid gland-specific gene selected from the group consisting of genes Nos.438 to 457 on FIGS. 52 to 53, or a set of genes consisting of two or more of human thyroid gland-specific genes selected from said group.

A human trachea-specific gene selected from the group consisting of genes Nos.458 to 467 on FIG. 53, or a set of genes consisting of two or more of human trachea-specific genes selected from said group.

A human lung-specific gene selected from the group consisting of genes Nos.468 to 491 on FIG. 53, or a set of genes consisting of two or more of human lung-specific genes selected from said group.

A human chest-specific gene selected from the group consisting of genes Nos.492 to 505 on FIGS. 53 to 54, or asset of genes consisting of two or more of human chest-specific genes selected from said group.

A human skin-specific gene selected from the group consisting of genes Nos.506 to 577 on FIGS. 54 to 56, or a set of genes consisting of two or more of human skin-specific genes selected from said group.

A human skeletal muscle-specific gene selected from the group consisting of genes Nos.578 to 650 on FIGS. 56 to 58, or asset of genes consisting of two or more of human skeletal muscle-specific genes selected from said group.

A human heart-specific gene selected from the group consisting of genes Nos.651 to 679 on FIG. 58, or a set of genes consisting of two or more of human heart-specific genes selected from said group.

A human liver-specific gene selected from the group consisting of genes Nos.680 to 852 on FIGS. 58 to 63, or a set of genes consisting of two or more of human liver-specific genes selected from said group.

A human spleen-specific gene selected from the group consisting of genes Nos.853 to 875 on FIGS. 63 to 64, or a set of genes consisting of two or more of human spleen-specific genes selected from said group.

A human kidney-specific gene selected from the group consisting of genes Nos.876 to 907 on FIG. 64, or a set of genes consisting of two or more of human kidney-specific genes selected from said group.

A human adrenal gland-specific gene selected from the group consisting of genes Nos.908 to 935 on FIGS. 64 to 65, or a set of genes consisting of two or more of human adrenal gland-specific genes selected from said group.

A human pancreas-specific gene selected from the group consisting of genes Nos.936 to 964 on FIGS. 65 to 66, or a set of genes consisting of two or more of human pancreas-specific genes selected from said group.

A human stomach-specific gene selected from the group consisting of genes Nos.965 to 985 on FIG. 66, or a set of genes consisting of two or more of human stomach-specific genes selected from said group.

A human small intestine-specific gene selected from the group consisting of genes Nos.986 to 1021 on FIGS. 66 to 67, or a set of genes consisting of two or more of human small intestine-specific genes selected from said group.

A human large intestine-specific gene selected from the group consisting of genes Nos.1022 to 1034 on FIGS. 67 to 68, or a set of genes consisting of two or more of human large intestine-specific genes selected from said group.

A human urinary bladder-specific gene selected from the group consisting of genes Nos.1035 to 1044 on FIG. 68, or a set of genes consisting of two or more of human urinary bladder-specific genes selected from said group.

A human prostate-specific gene selected from the group consisting of genes Nos.1045 to 1052 on FIG. 68, or a set of genes consisting of two or more of human prostate-specific genes selected from said group.

A human testis-specific gene selected from the group consisting of genes Nos.1053 to 1459 on FIGS. 68 to 79, or a set of genes consisting of two or more of human testis-specific genes selected from said group.

A human ovary-specific gene selected from the group consisting of genes Nos.1460 to 1466 on FIG. 79, or a set of genes consisting of two or more of human ovary-specific genes selected from said group.

A human placenta-specific gene selected from the group consisting of genes Nos.1467 to 1561 on FIGS. 79 to 82, or a set of genes consisting of two or more of human placenta-specific genes selected from said group.

A human uterus-specific gene selected from the group consisting of genes Nos.1562 to 1572 on FIG. 82, or a set of genes consisting of two or more of human uterus-specific genes selected from said group.

A human bone marrow-specific gene selected from the group consisting of genes Nos.1573 to 1647 on FIGS. 82 to 84, or a set of genes consisting of two or more of human bone marrow-specific genes selected from said group.

A human fetal brain-specific gene selected from the group consisting of genes Nos.1648 to 1678 on FIGS. 84 to 85, or a set of genes consisting of two or more of human fetal brain-specific genes selected from said group.

A human fetal liver-specific gene selected from the group consisting of genes Nos.1679 to 1704 on FIG. 85, or a set of genes consisting of two or more of human fetal liver-specific genes selected from said group.

The invention also provides a transcription product of any of the above-mentioned tissue-specific genes and a set of transcription products thereof, as well as an oligonucleotide probe and a set thereof.

The invention also provides a DNA microarray carrying the set of tissue-specific genes' transcription products or the set of probe.

Thus, the inventors employed a GeneChip U133 microarray produced by Affymetrix to search 20708 genes in 35 normal tissues (FIG. 40) and finally identified 1189 genes (housekeeping genes) shown in FIGS. 1 to 39. At the same time, the inventors identified (a group of) genes which are expressed specifically in respective 35 normal tissues (FIGS. 41 to 85).

In FIGS. 1 to 31, the left column represents gene numbers, Column A represents GeneChip U133 probe sets, Column B represents gene names, Column C represents UniGene IDs, Column D represents GeneBank (URL:http://www.ncbi.nlm.nih.gov/) register numbers, Column E represents gene abbreviations, Column F represents gene-locating chromosome numbers, Column G represents statistically obtained mean values, and Column H represents coefficients of variance (CV). A smaller “CV value” corresponds to a higher similarity in the gene expression degree in each tissue, and in FIGS. 1 to 39 a gene is listed up in an order from smaller CV value (i.e. higher similarity of gene expression)

In FIGS. 41 to 85, the left column represents gene numbers, Column A represents tissues, Column B represents GeneChip U133 probe sets, Column C represents gene names, Column D represents UniGene IDs, Column E represents GeneBank register numbers, Column F represents gene abbreviations, Column G represents gene-locating chromosome numbers, Column H represents tissue-specific scores, Column I represents expression frequency values in respective tissues, Column J represents logarithmic expression values in respective tissues, Column K represents mean values in other tissues, and Column L represents standard deviations.

These data have been published since Apr. 16, 2002 (updated on Jul. 17, 2002) in SBM database published by the inventors via Internet (URL:http://www2.genome.rcast.u-tokyo.ac jp/database/).

This invention is based on the gene populations as described above. As used herein, the term “gene” means a DNA sequence encoding the entire transcription product (including such as intron, expression regulatory sequence and the like), preferably an isolated and purified DNA sequence.

The term “transcription product” means an RNA molecule transcribed from a gene, as well as a protein or peptide translated from this RNA molecule. A cDNA synthesized artificially from RNA (mRNA) is also included in this transcription product.

The term “normal human tissue” means a human body tissue of a human (including a fetus) having no certain detected disease and the like.

The term “gene expression” is defined as that a gene transcription product (RNA) is present in a significantly larger amount (P<0.05, detection value P determined by statistical analysis mentioned below). Accordingly, any of the “housekeeping gene” is a gene whose detection value P is less than 0.05 over the entire 35 tissues. The term “tissue-specific gene” represents a gene whose detection value P in the single specific tissue is less than 0.05 and that in any of other 34 tissues is 0.05 or more.

The term “oligonucleotide probe” means a nucleotide sequence which hybridizes with an intended DNA fragment or RNA fragment on the basis of the sequence complementarily, and which has 6 to 100 nucleotide length. Nevertheless, it may have an appropriate length within the ranges, for example, from 100 to 200 nucleotides, 200 to 500 nucleotides.

Other terms and concepts employed in the invention are described in the following embodiments. Various gene engineering technologies and the like employed for conducting the invention can readily and surely be performed by those skilled in the art based for example on a known reference (for example, in Sambrook and Maniatis, Molecular Cloning, A Laboratory Manual, Cold Spring Harbor Laboratory Press, New York, 1989; Ausubel, F. M. et al., Current Protocols in Molecular Biology, John Wiley & Sons, New York, N.Y., 1995) except for those otherwise specified for their sources.

### BRIEF DESCRIPTION OF DRAWINGS

FIGS. 1 to 39 show the lists of the housekeeping genes provided by the invention.

FIG. 40 shows the list of the normal human tissues subjected in the invention.

FIGS. 41 to 85 show the lists of the tissue-specific genes provided by the invention.

### BEST MODE FOR CARRYING OUT THE INVENTION

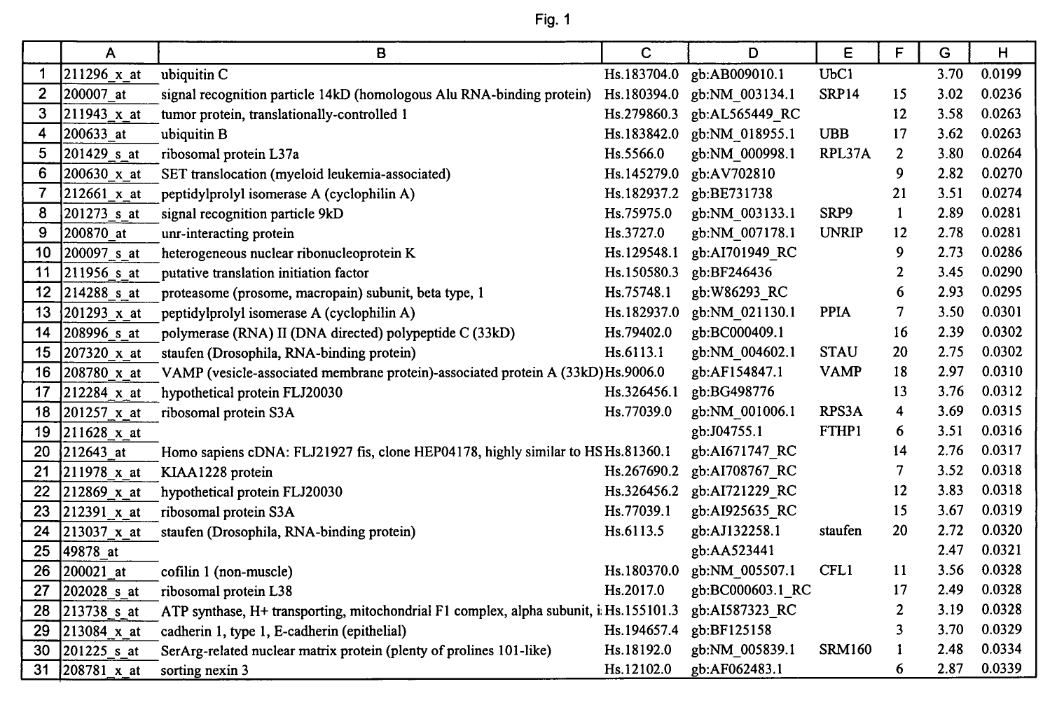

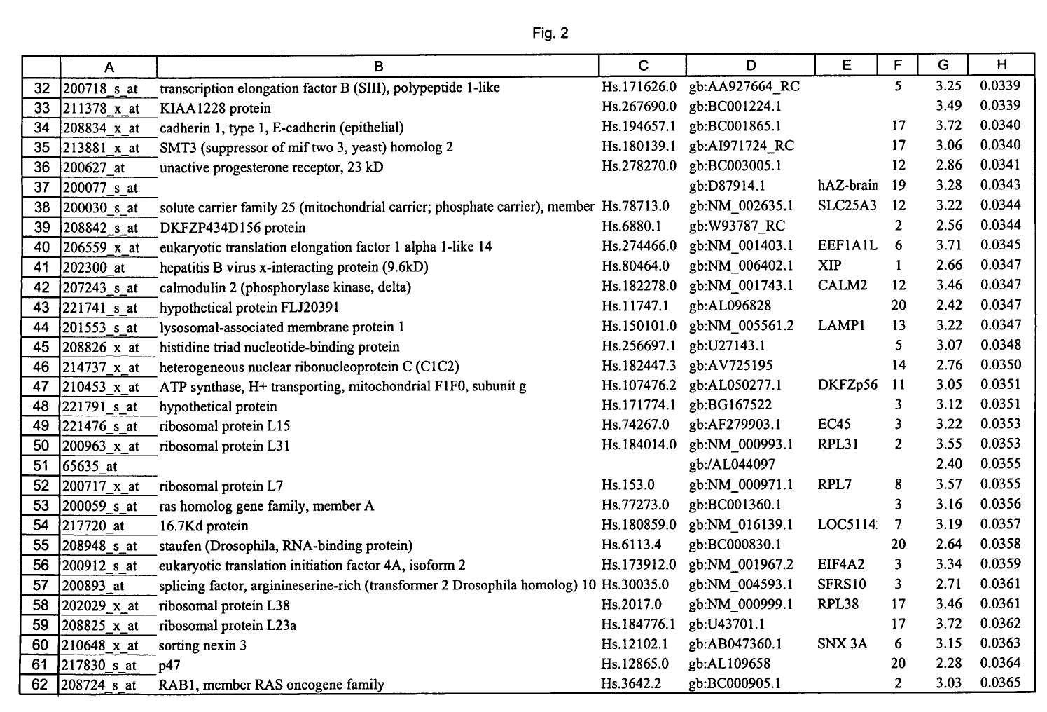

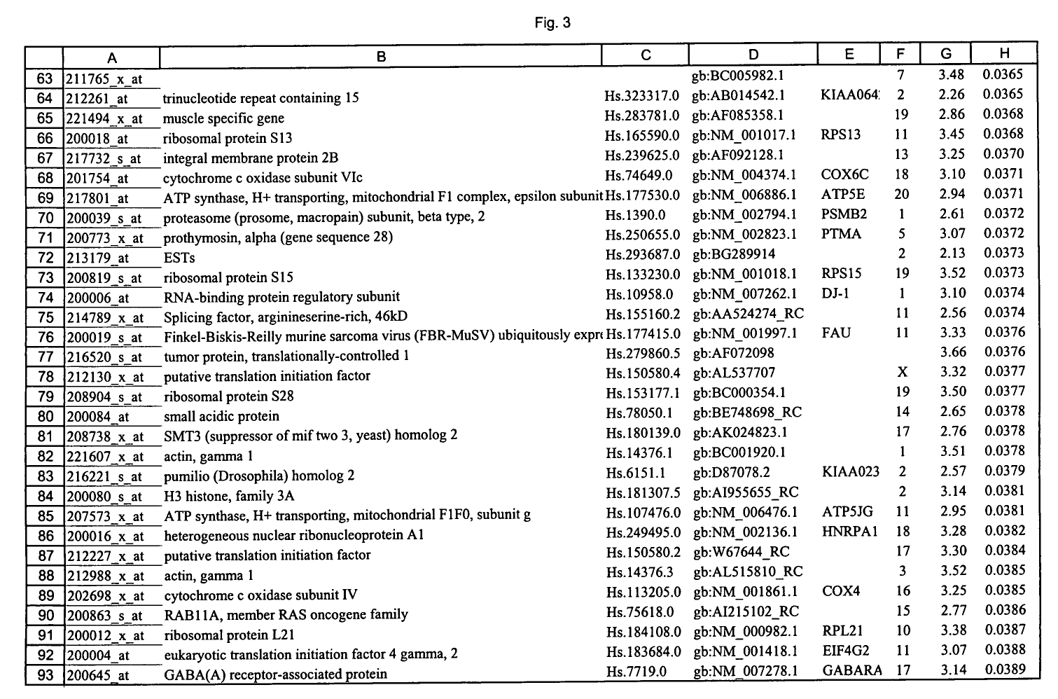

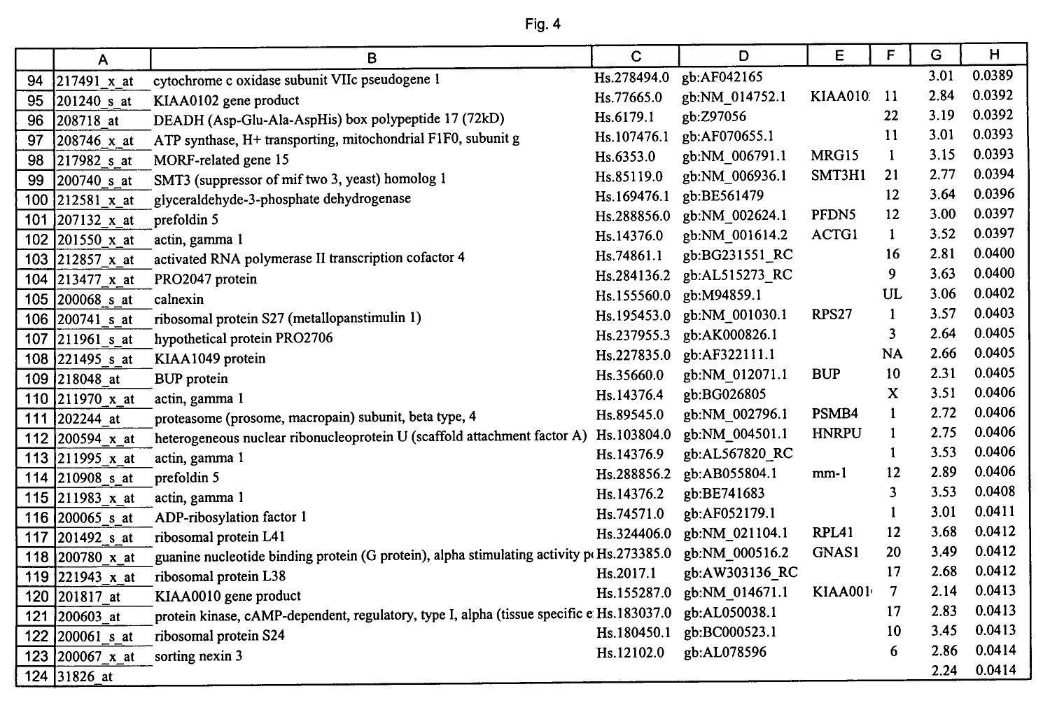

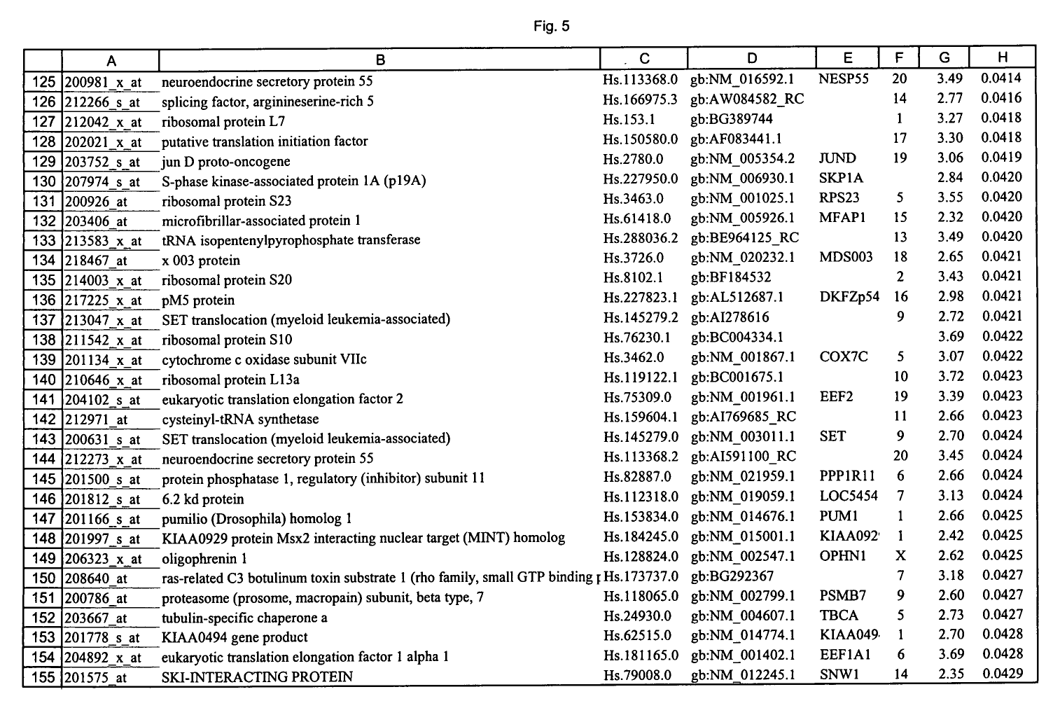

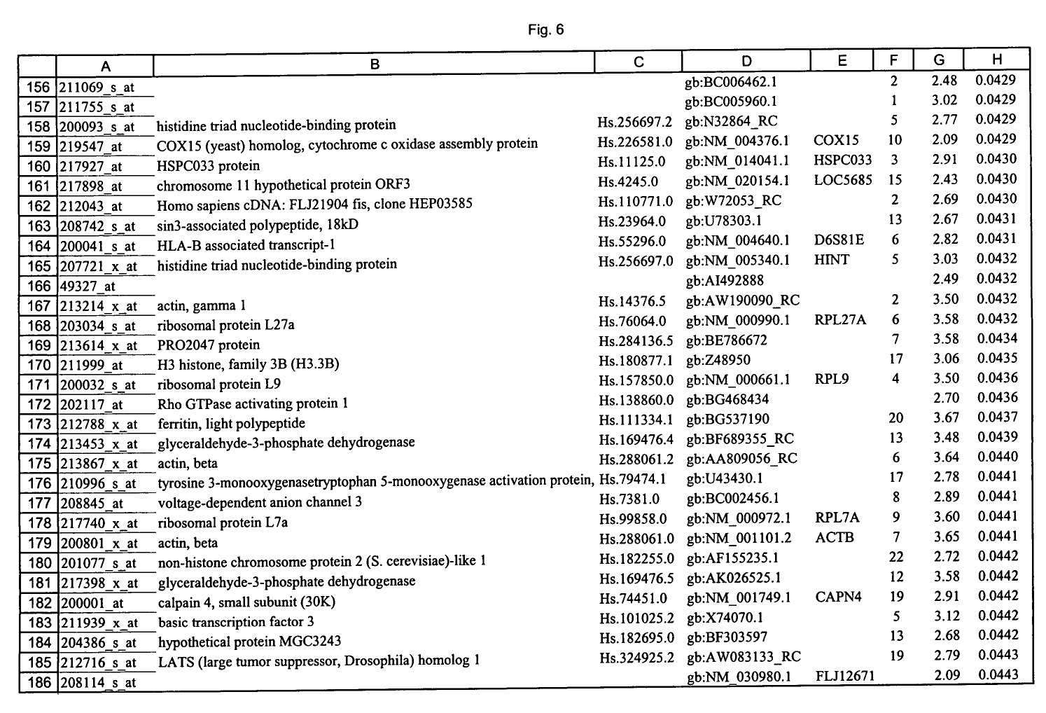

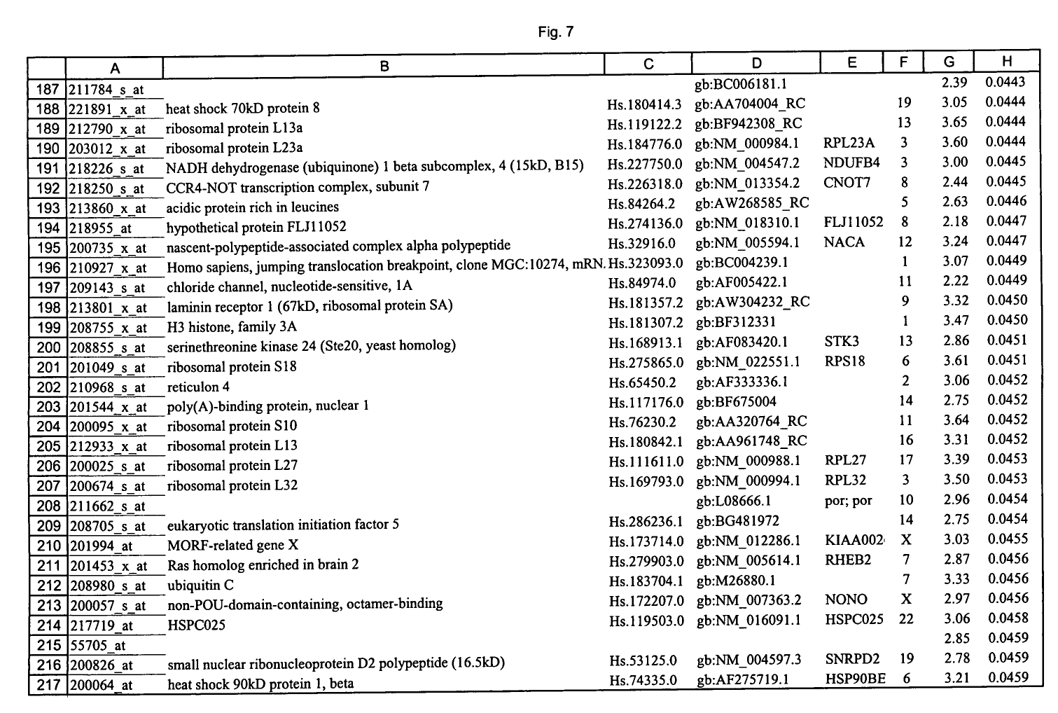

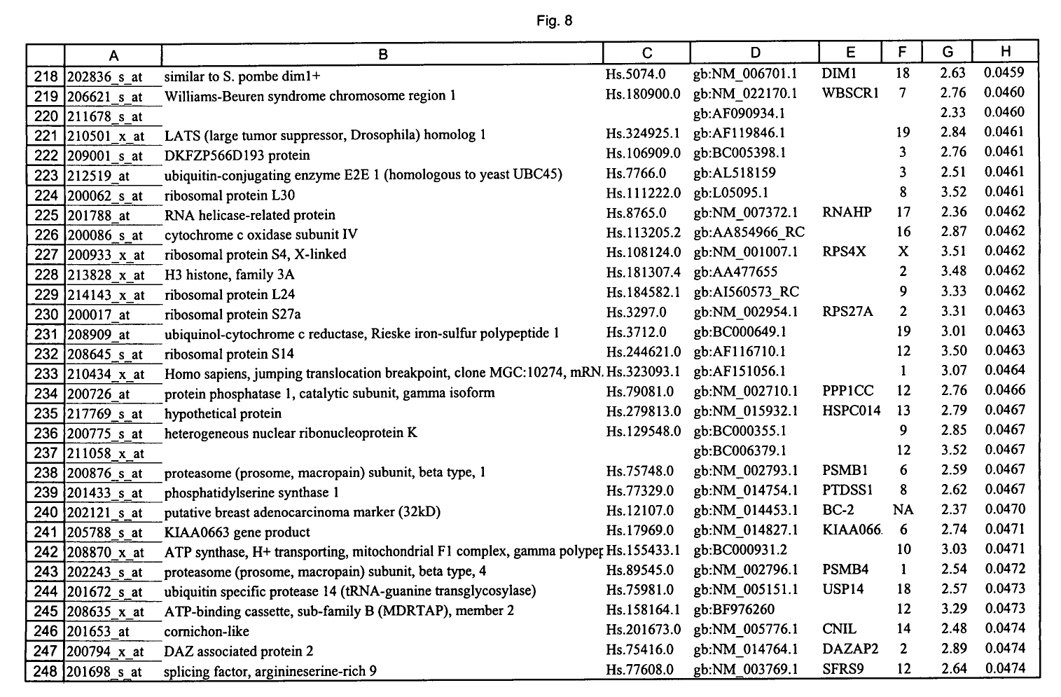

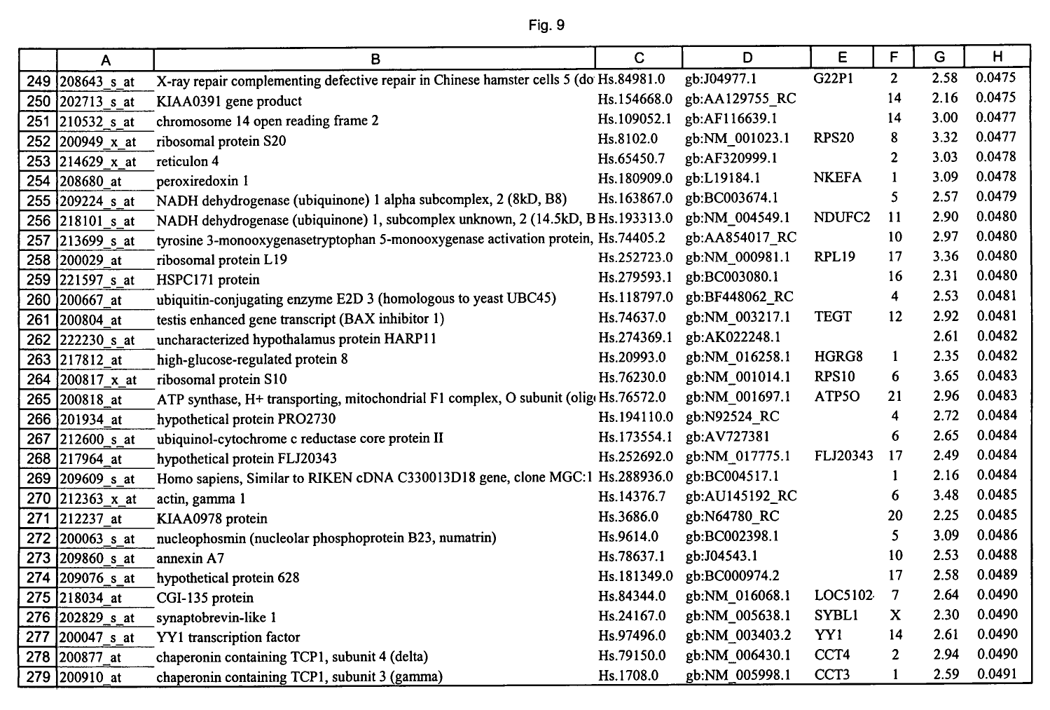

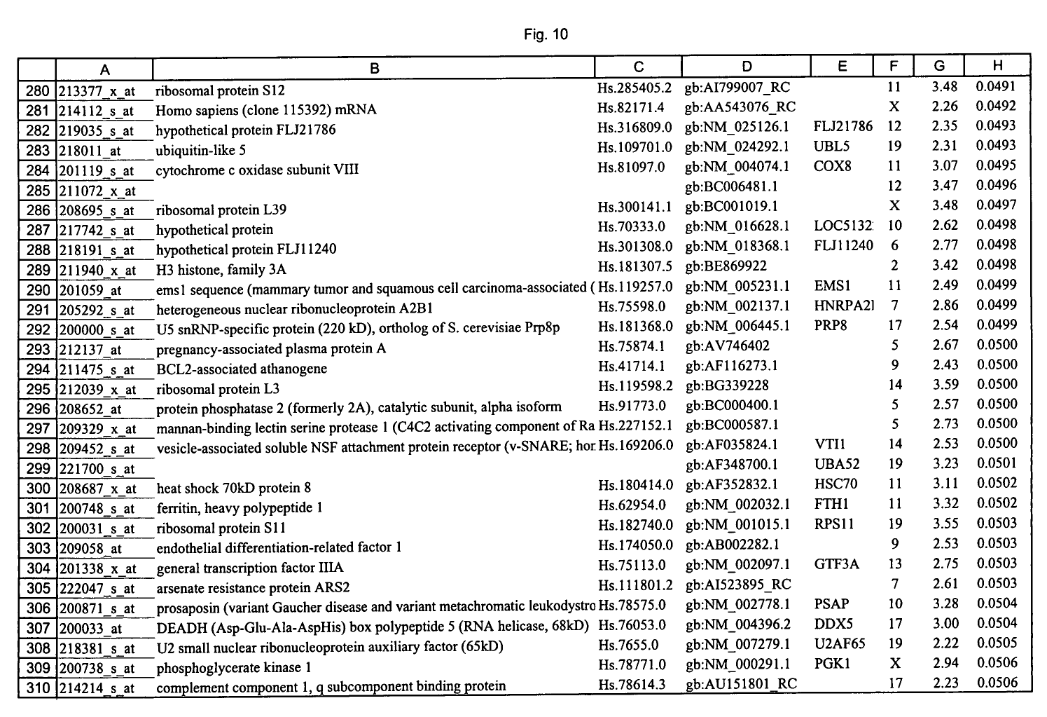

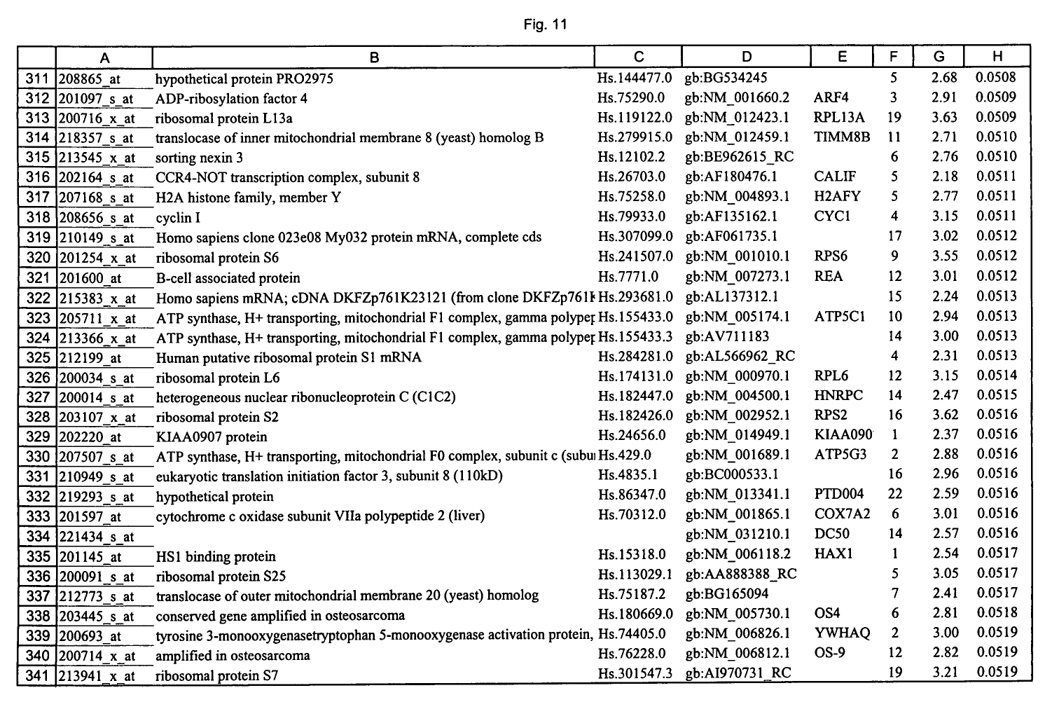

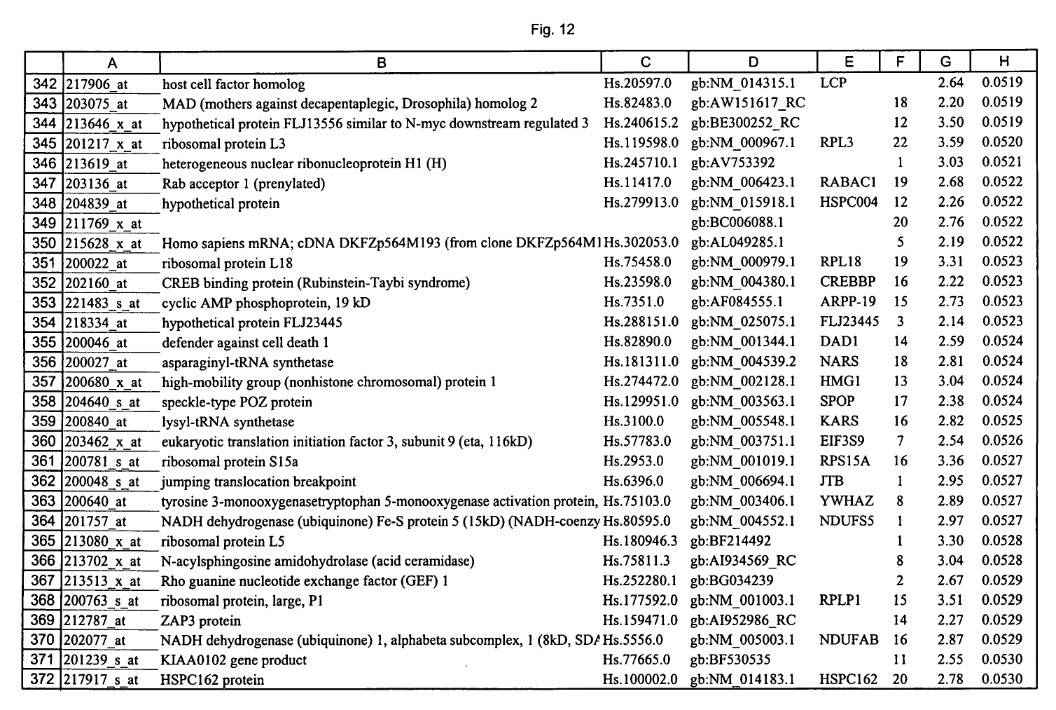

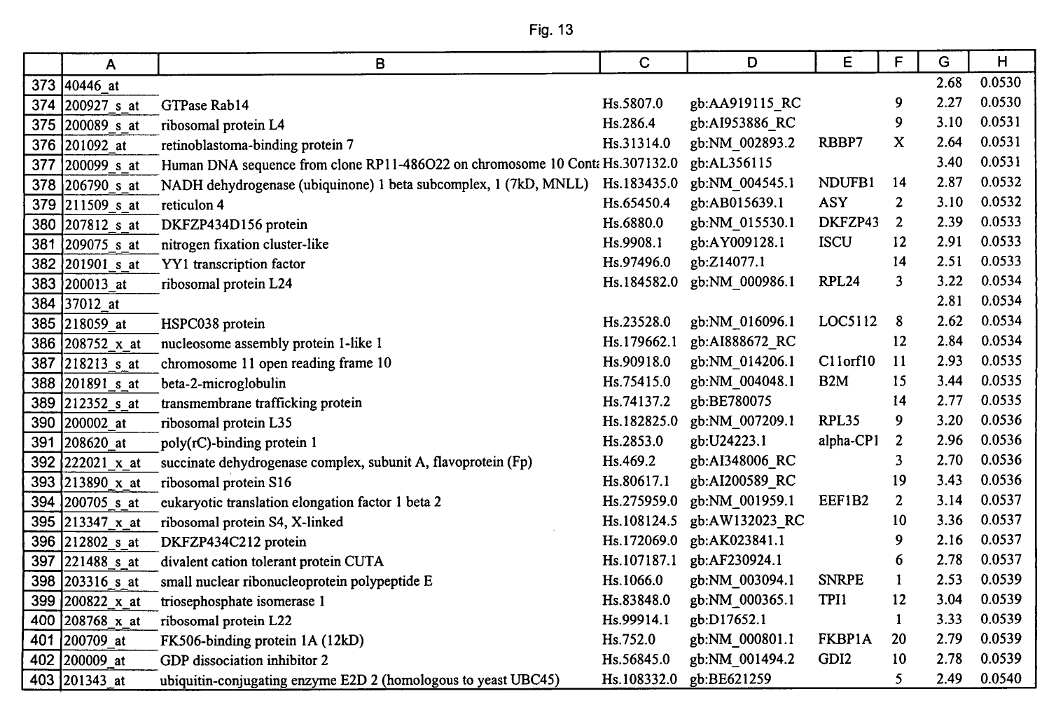

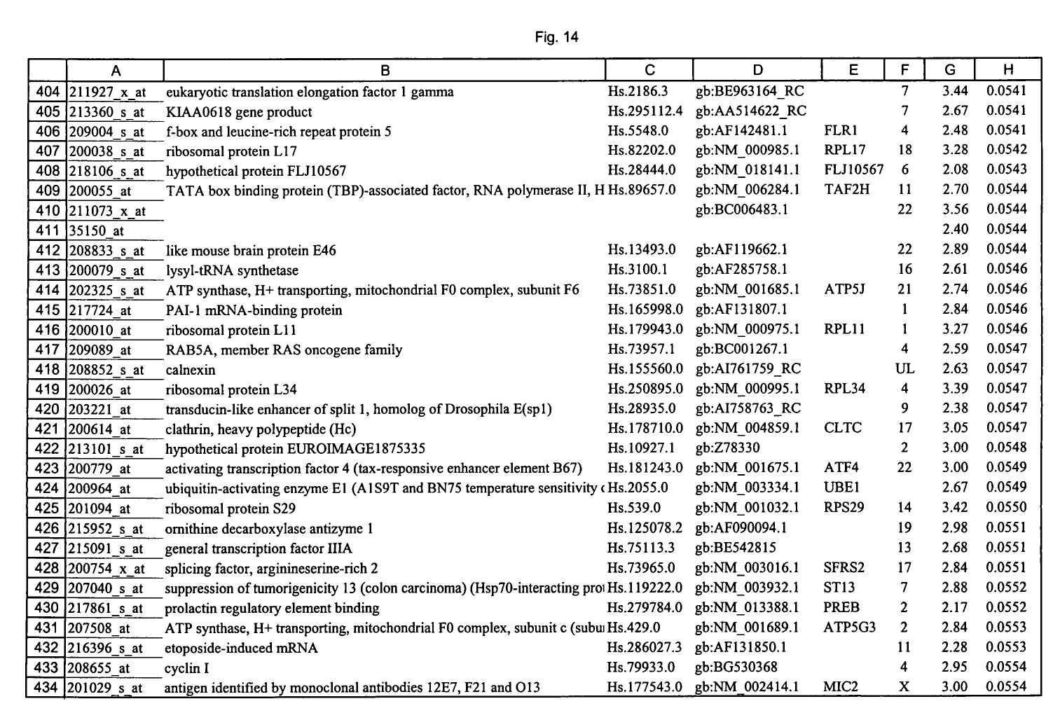

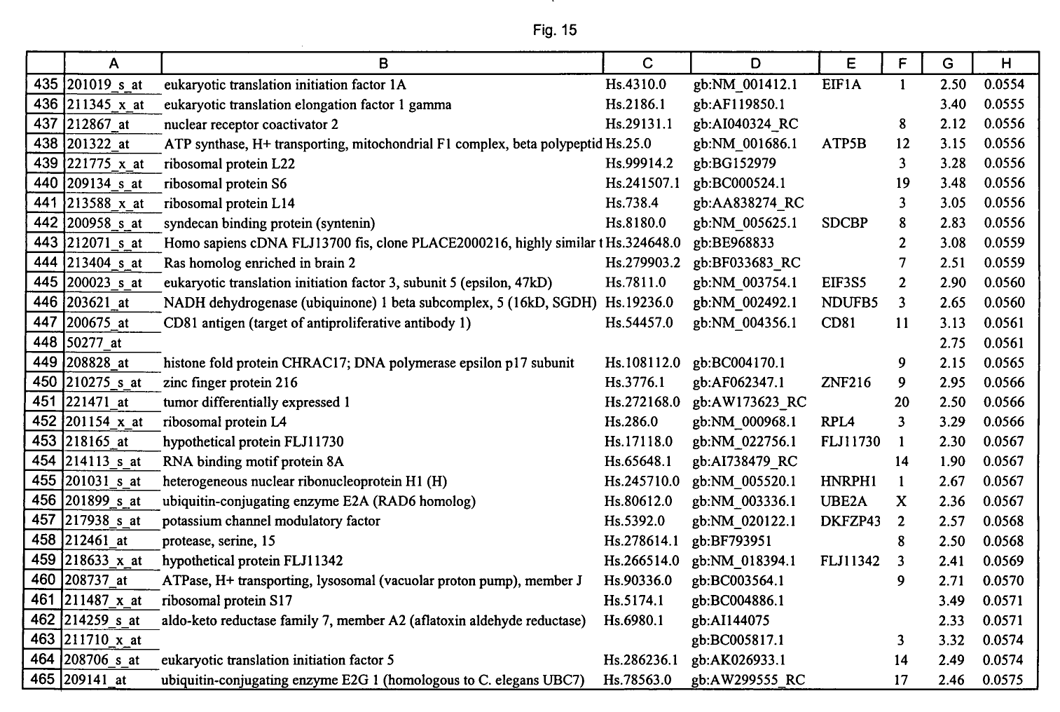

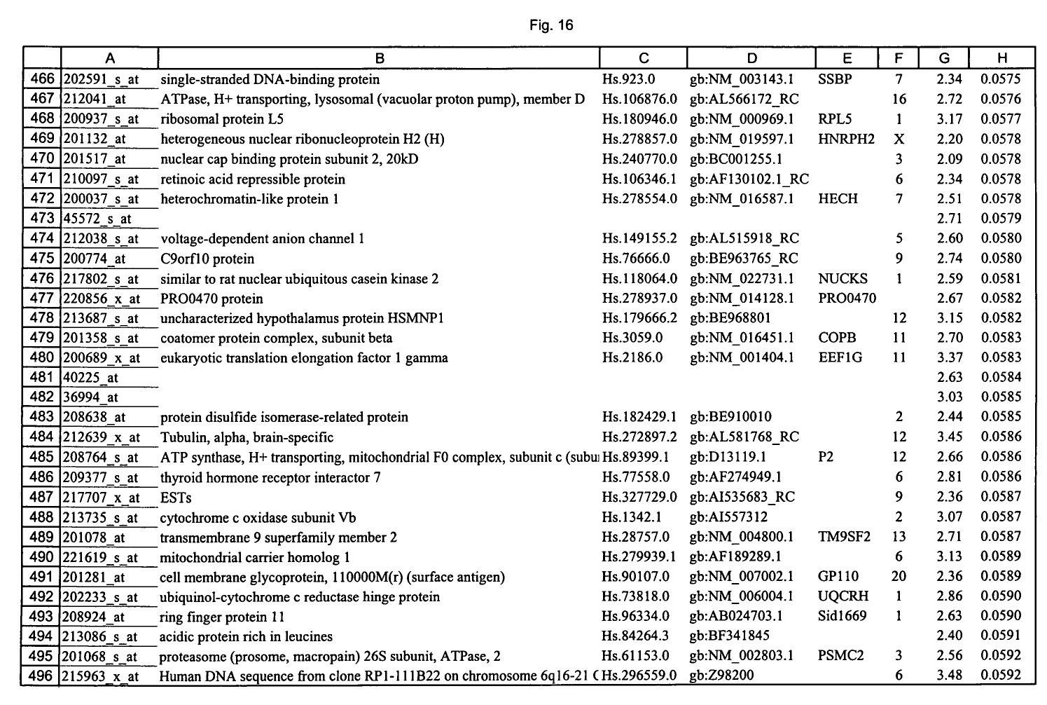

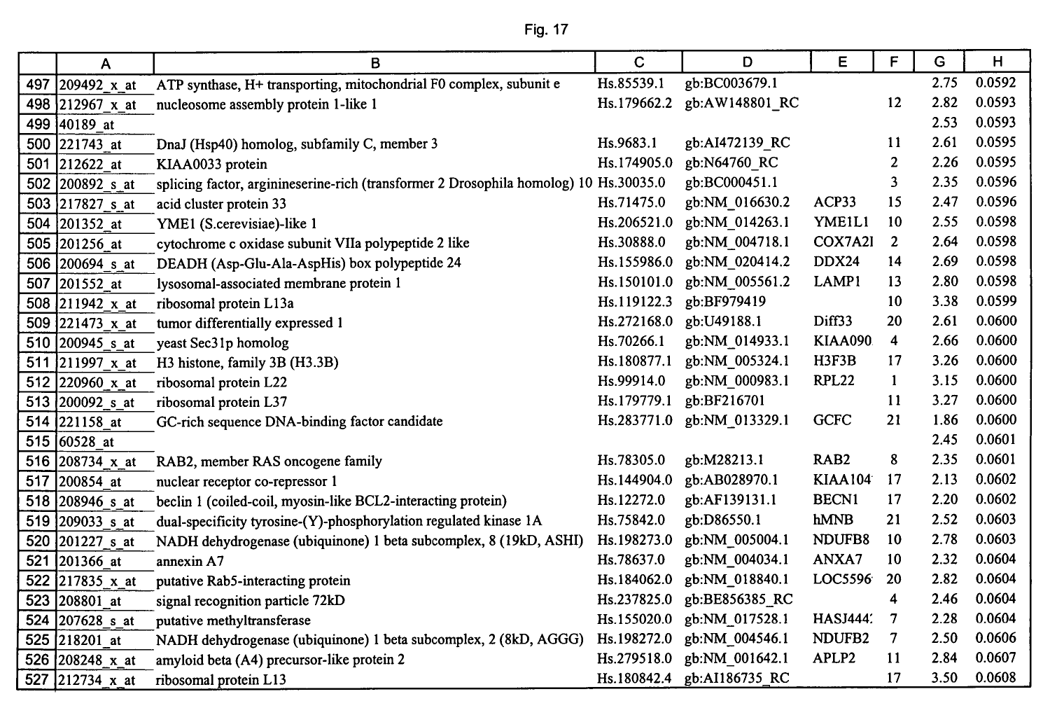

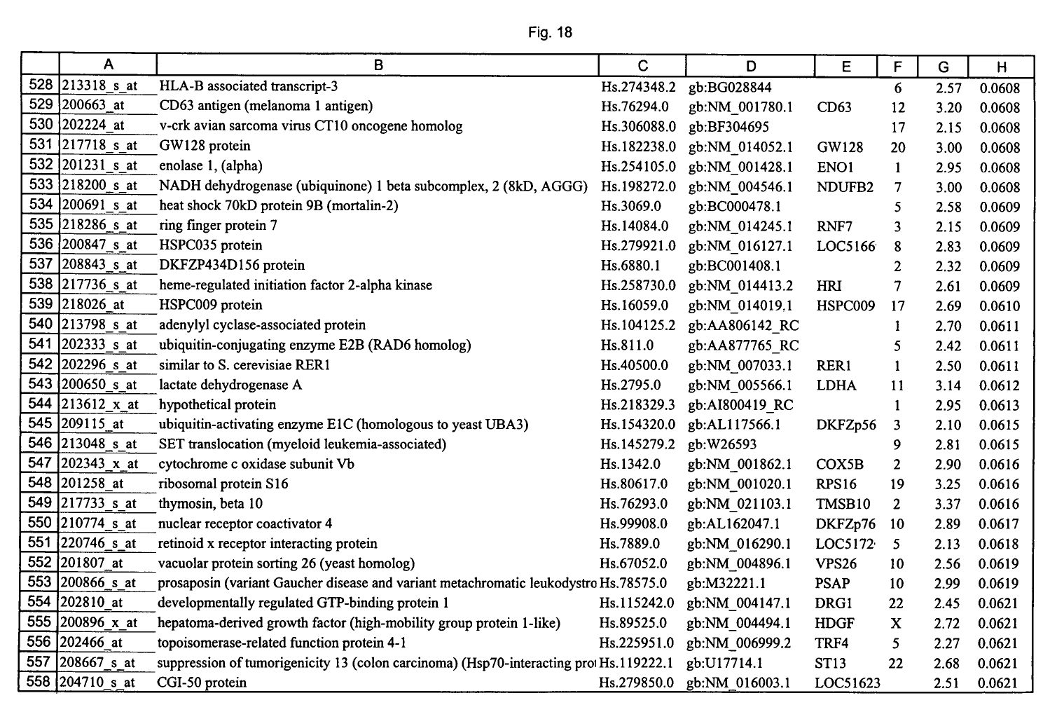

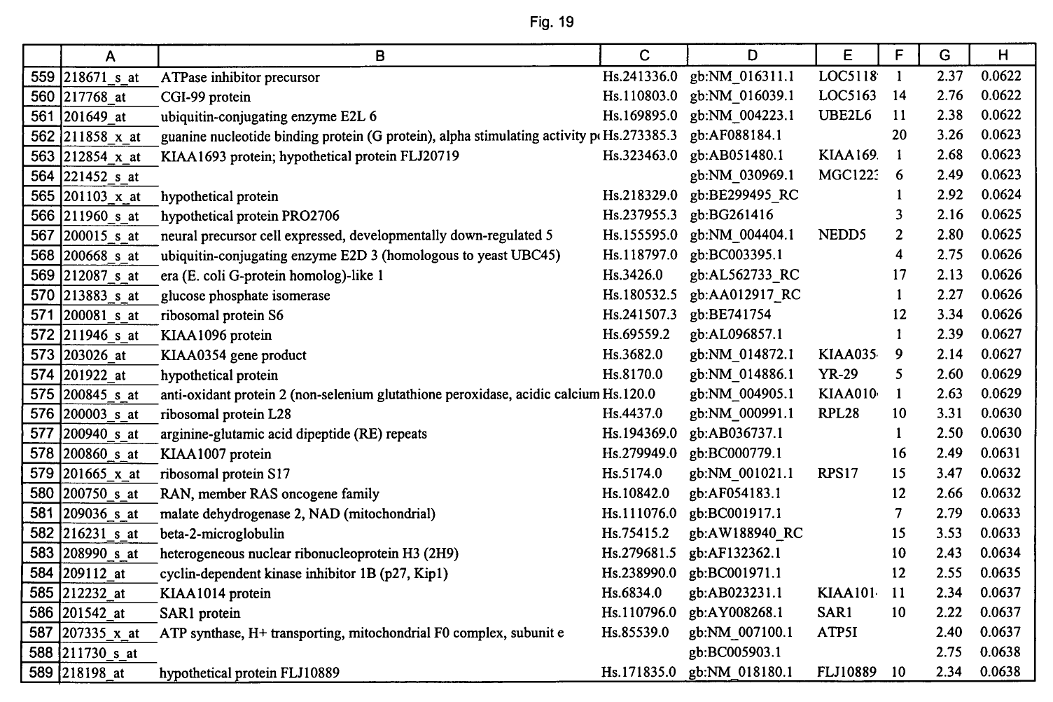

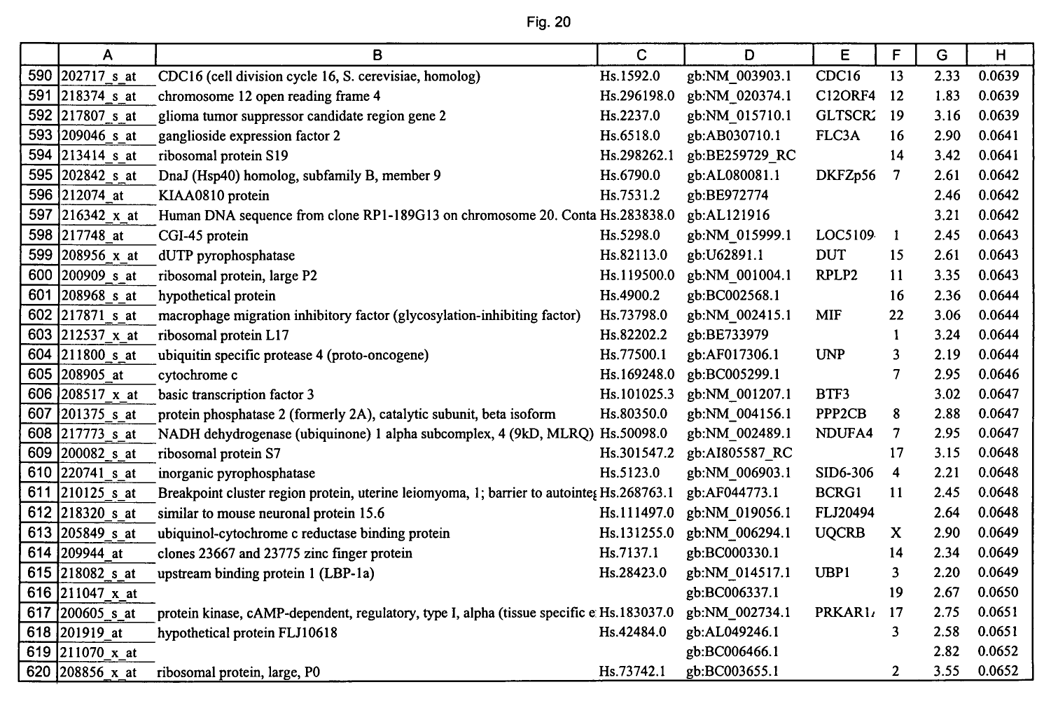

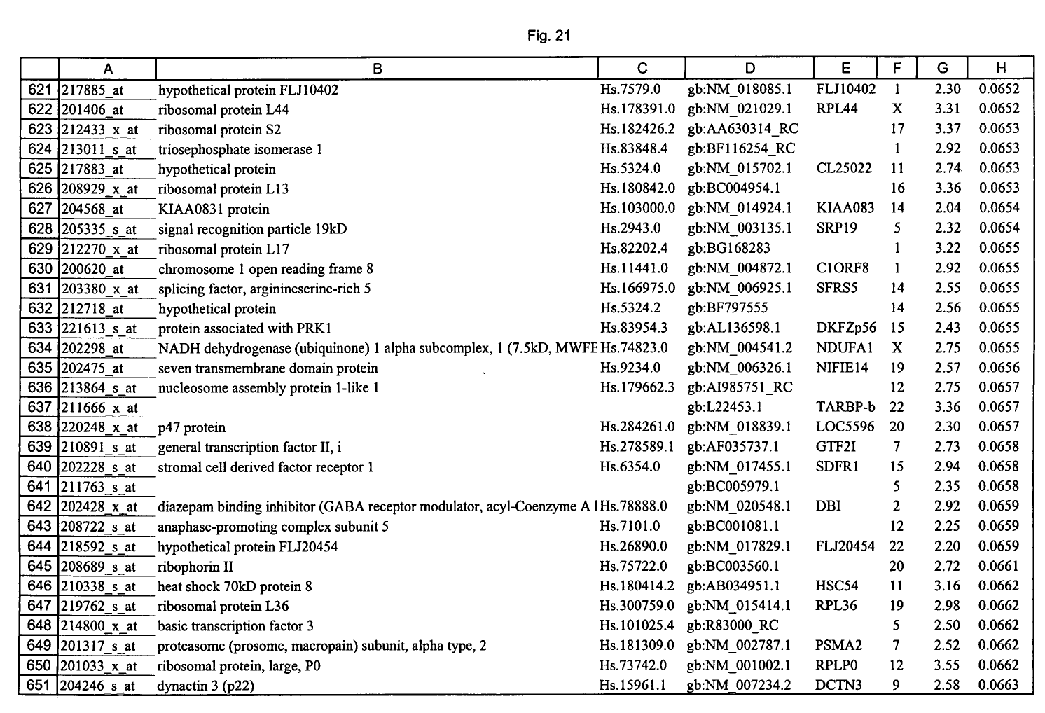

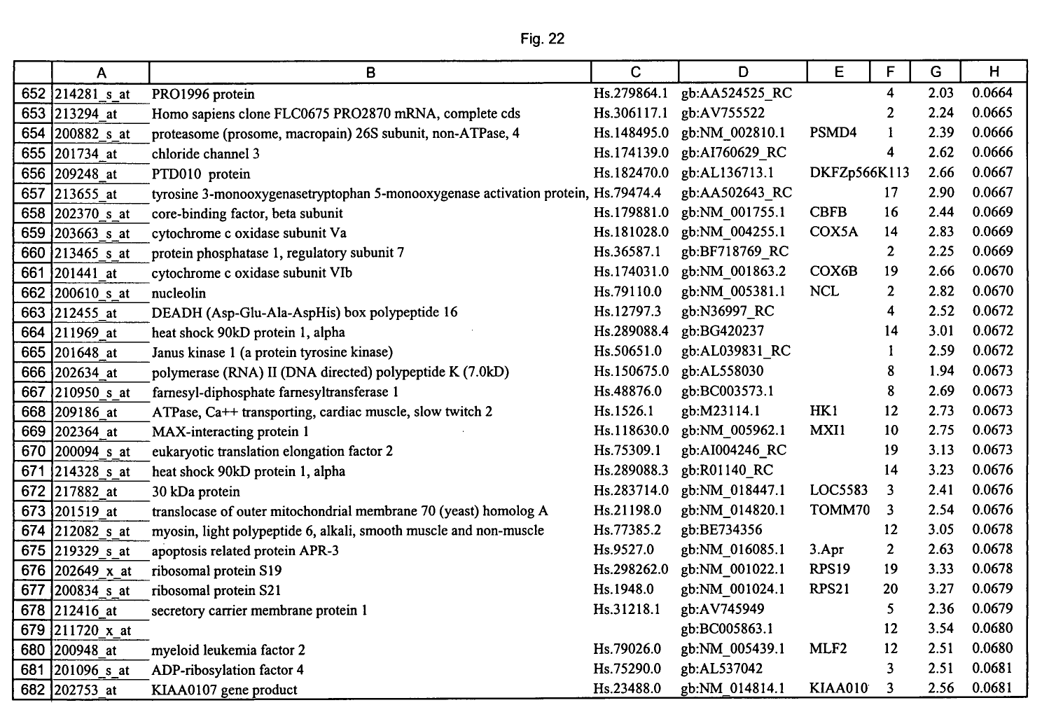

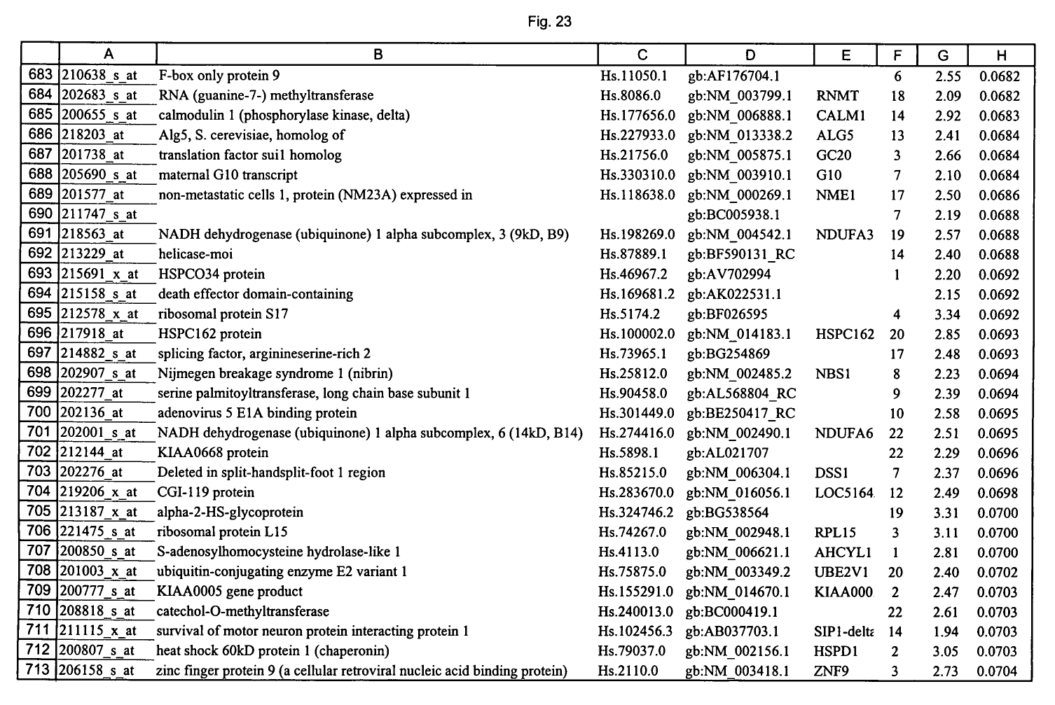

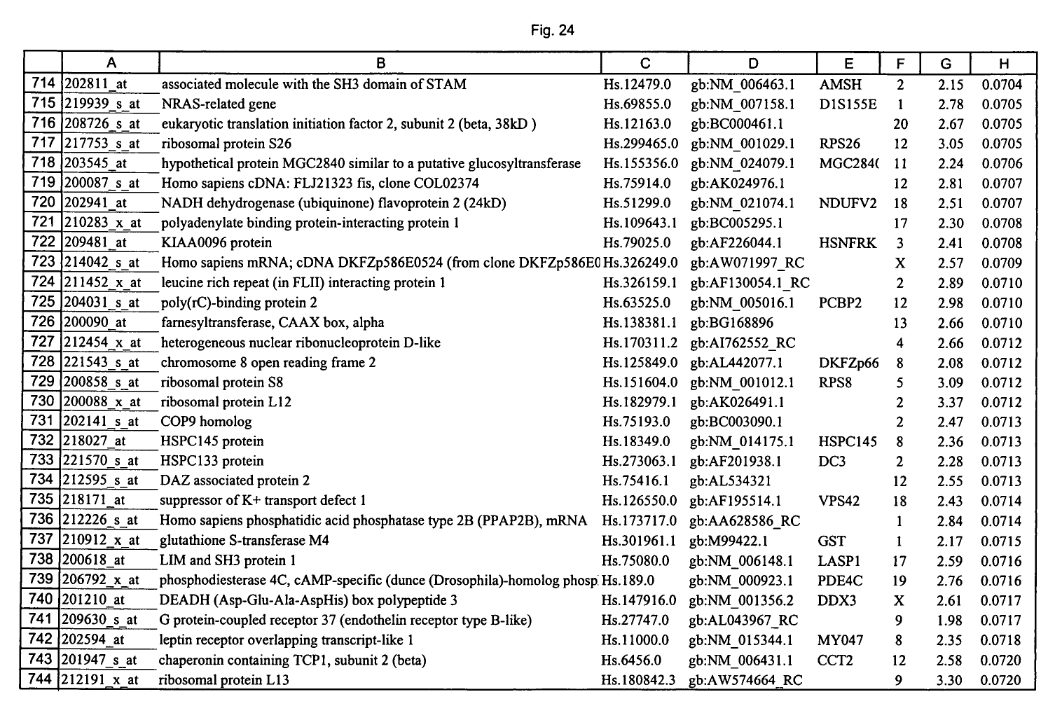

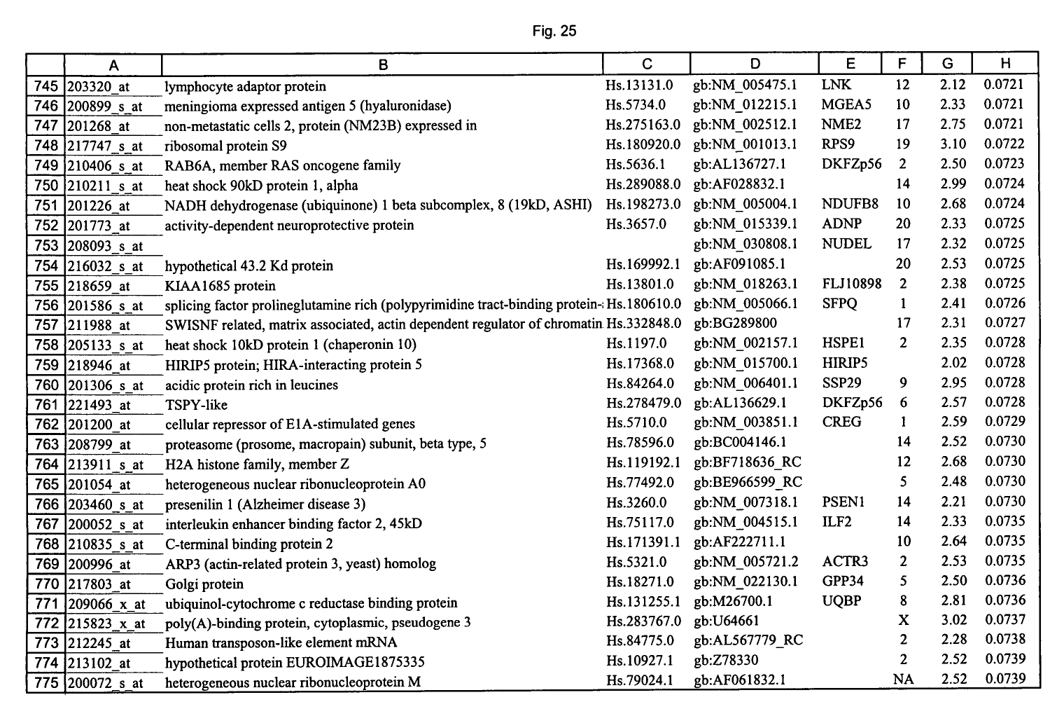

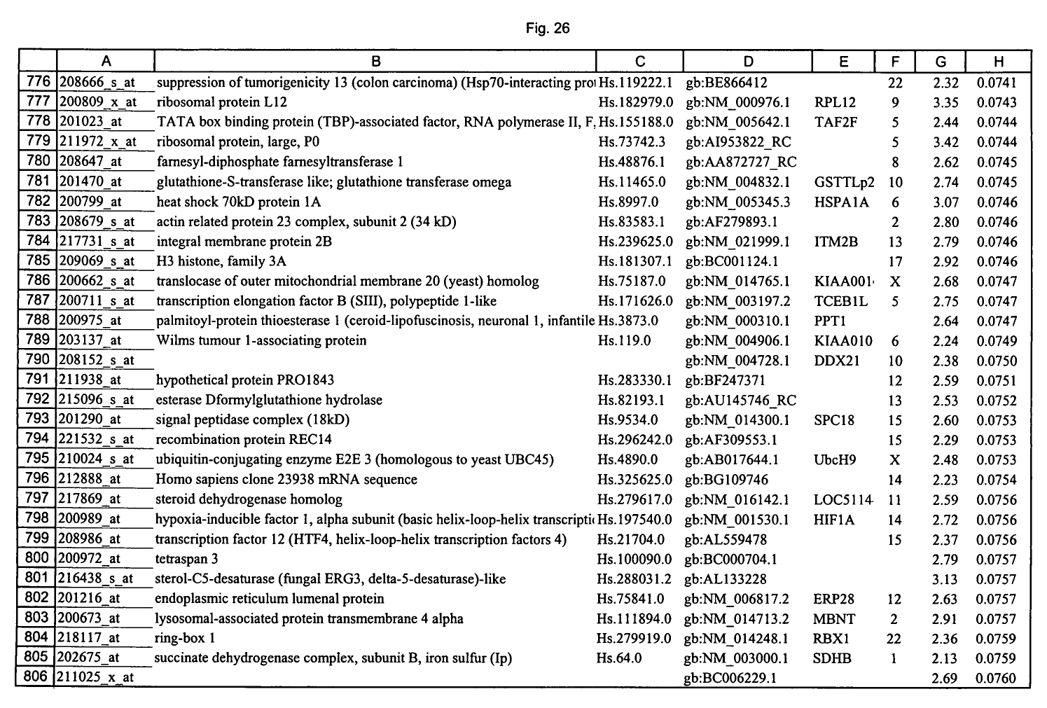

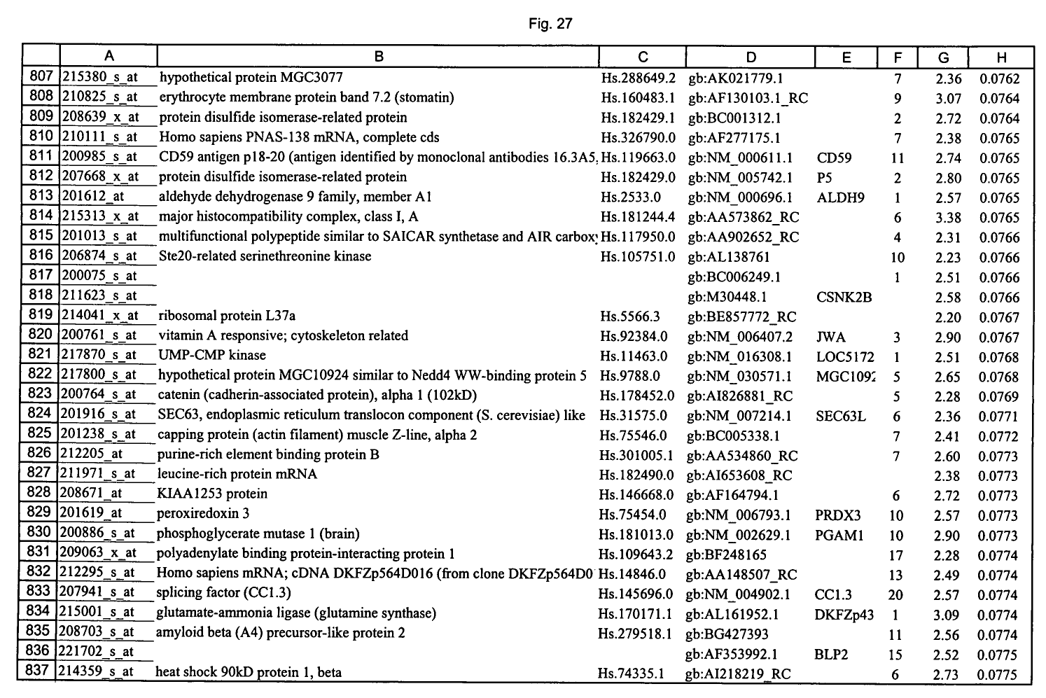

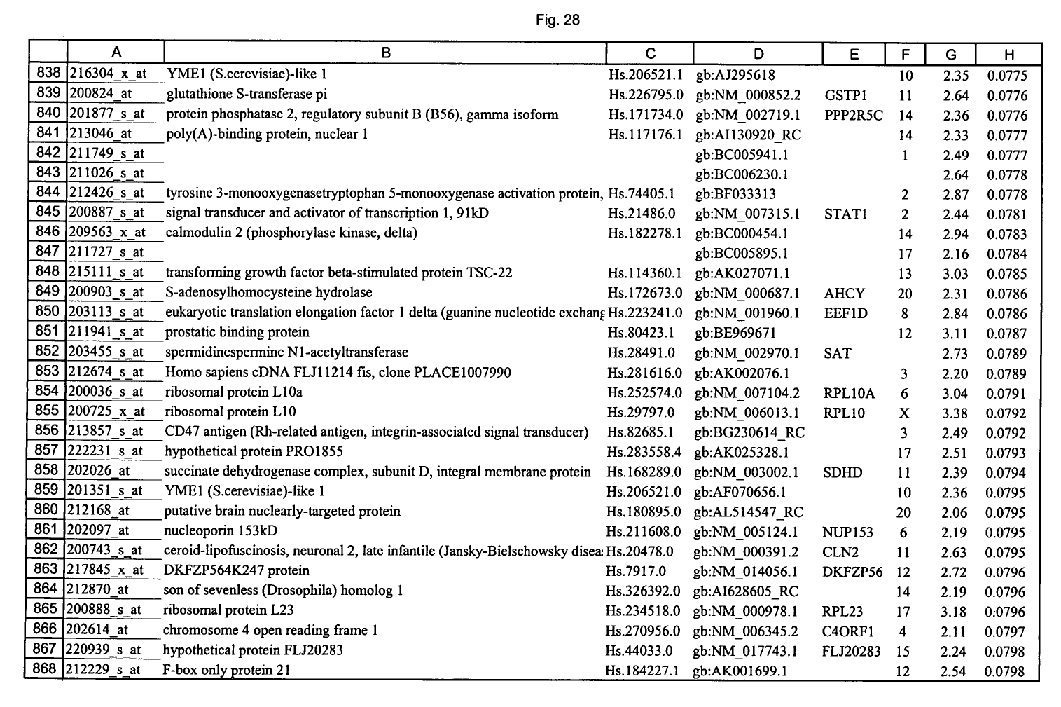

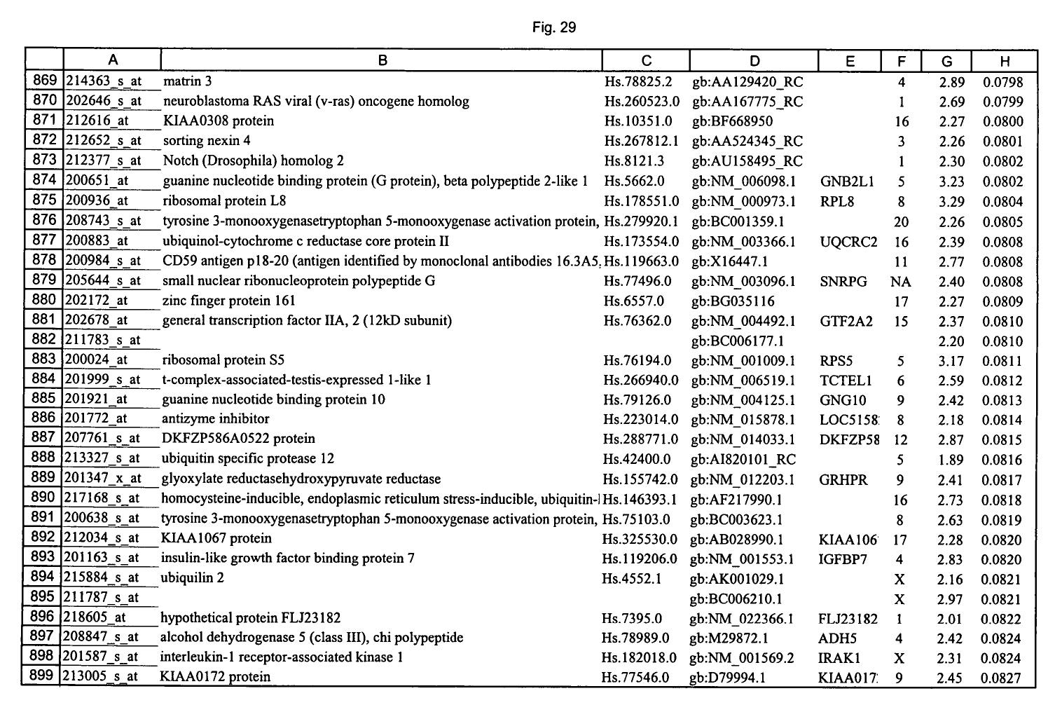

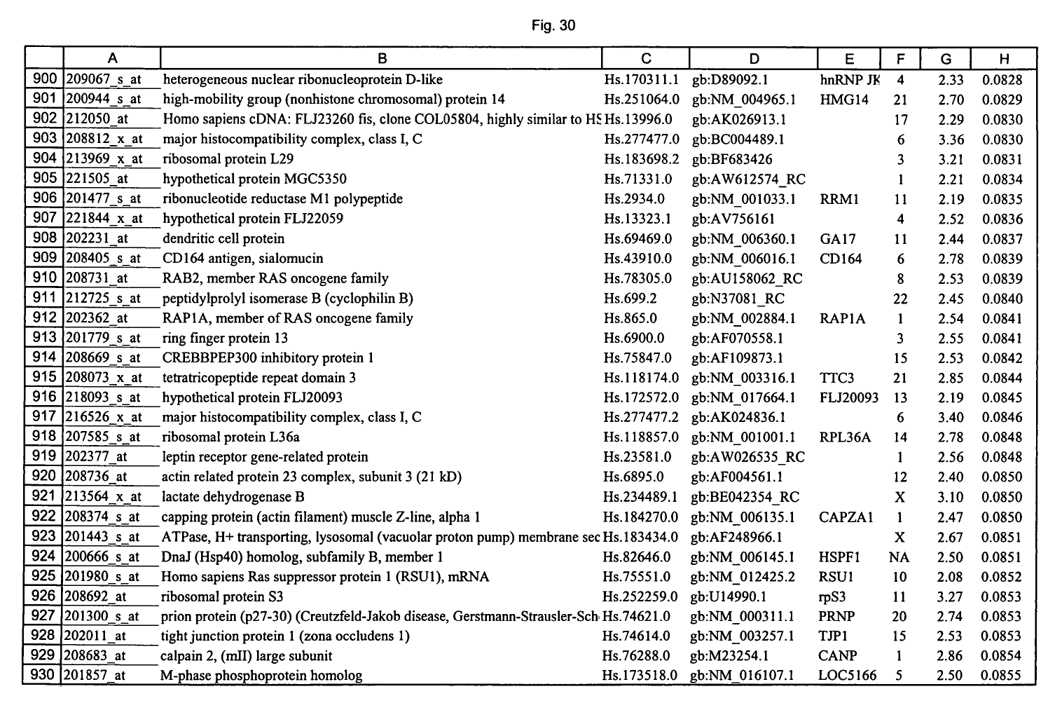

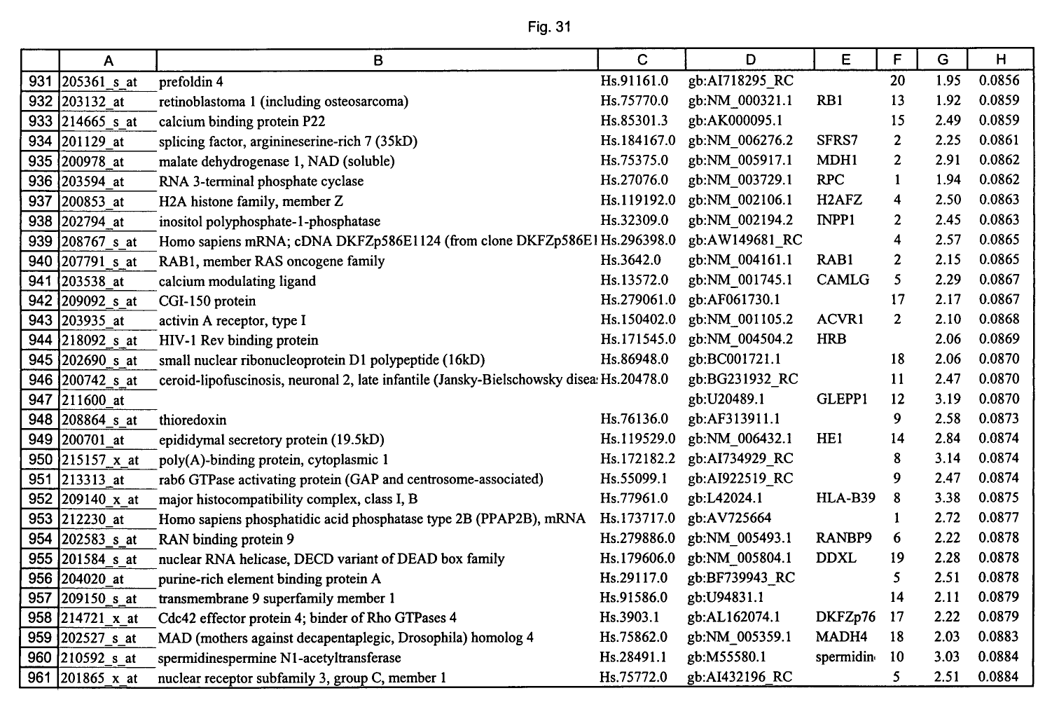

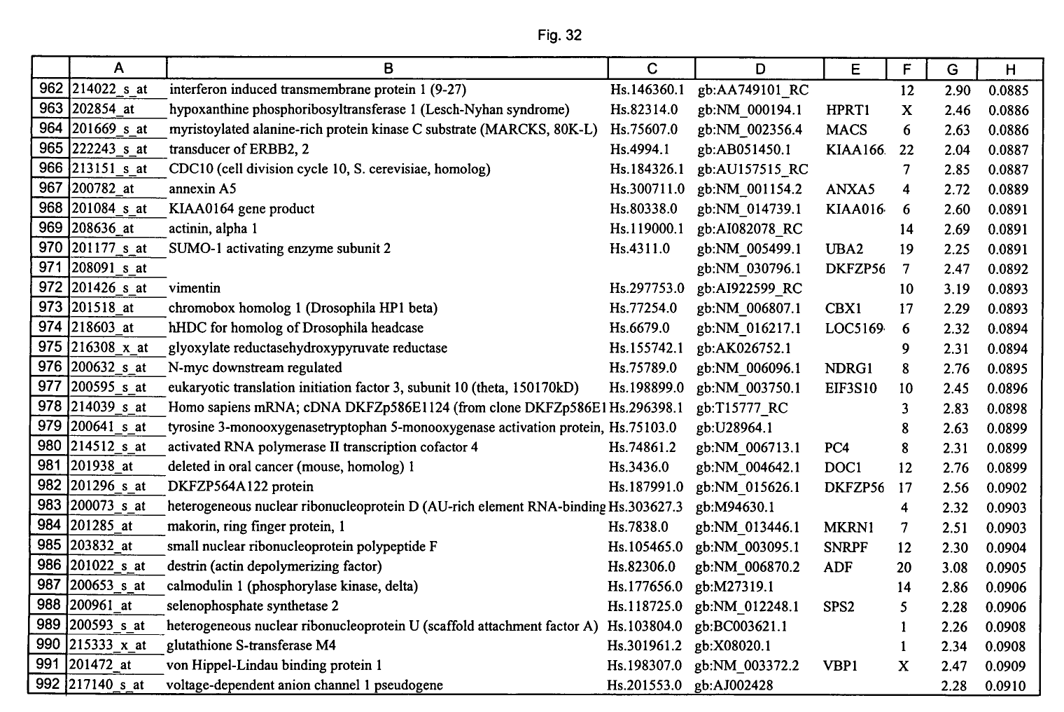

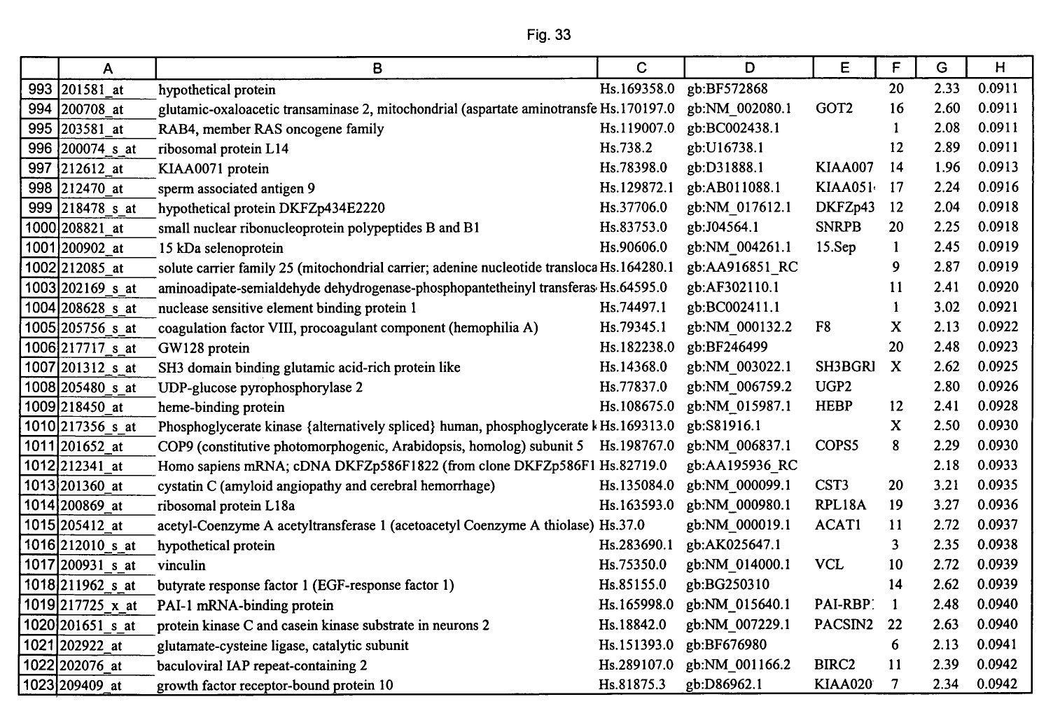

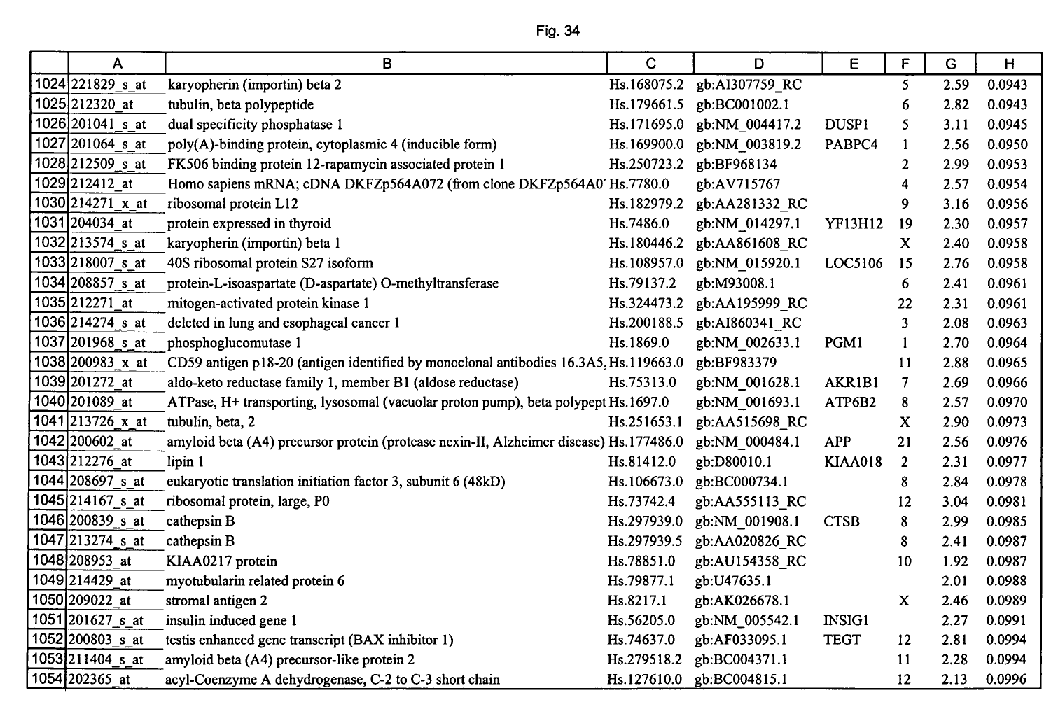

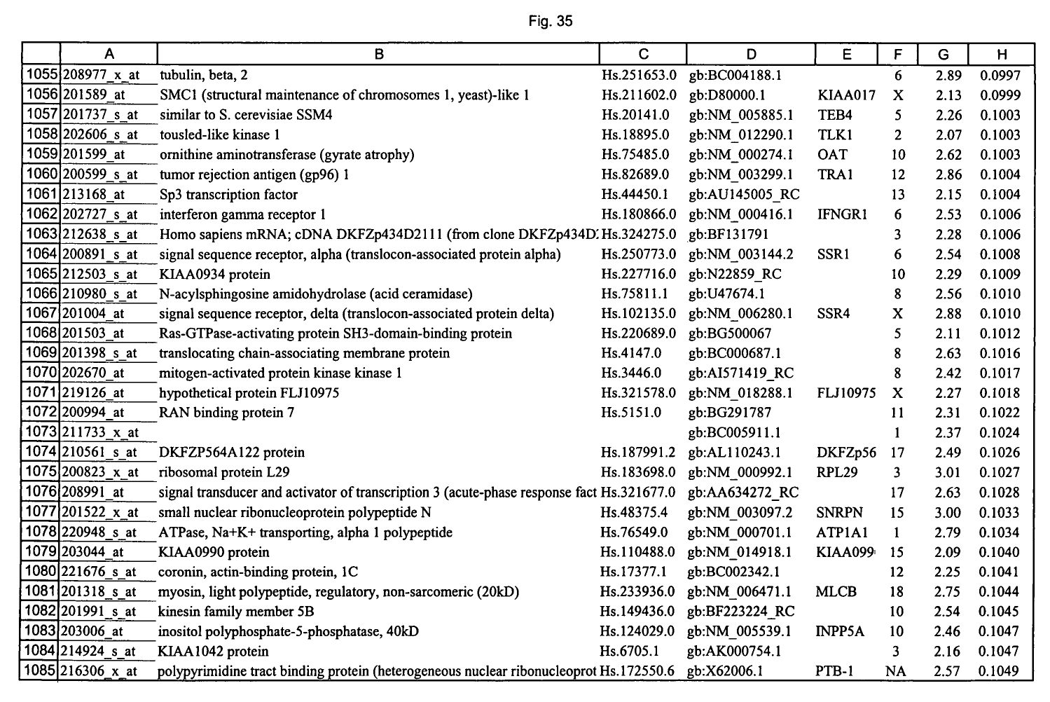

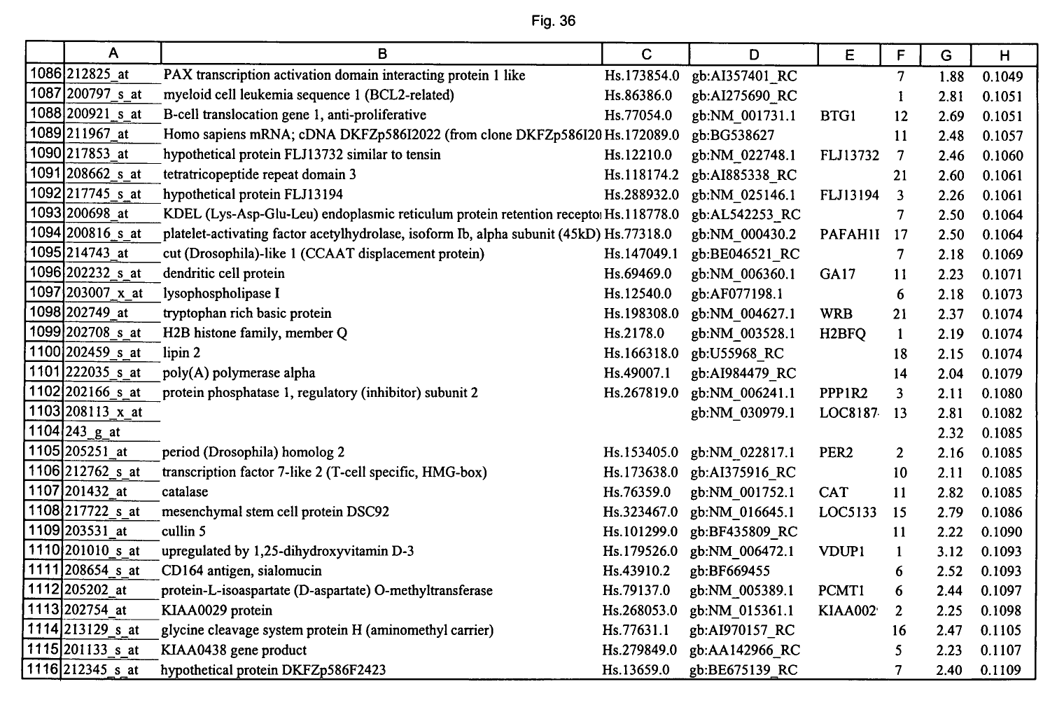

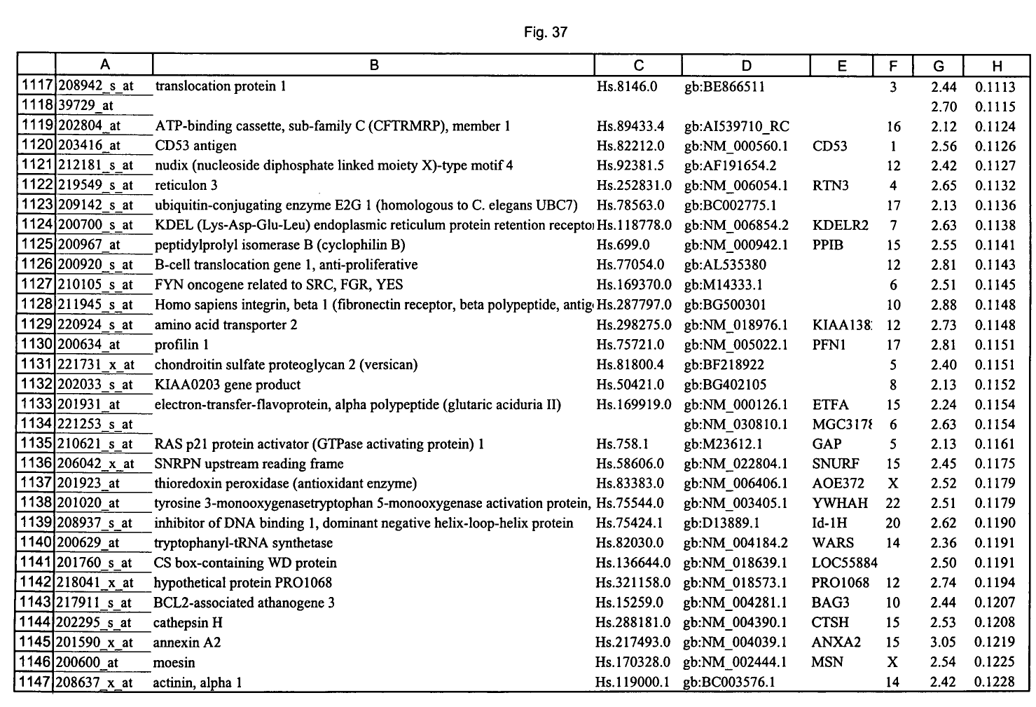

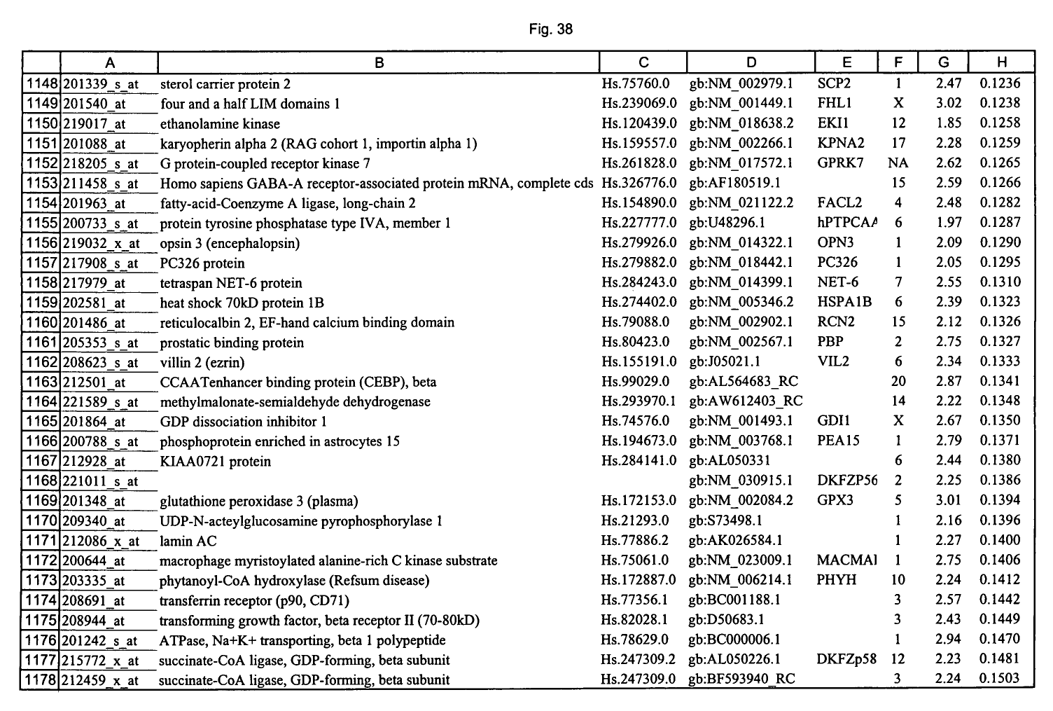

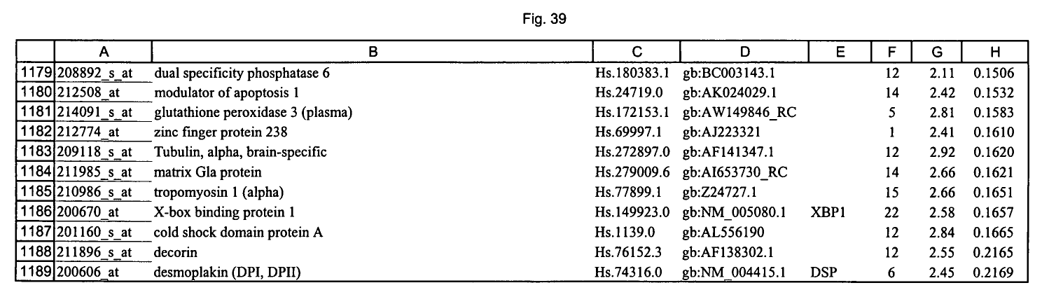

The sequences (genome sequences, mRNA sequences and the like) of the housekeeping genes of the invention are well-known, registered under the GeneBank database register numbers shown in Column D in FIGS. 1 to 39, and the genes of No. 1 to 200 shown in FIGS. 1 to 7 have the respective cDNA sequences represented by SEQ ID Nos.1 to 200. Accordingly, any of these housekeeping genes can be isolated by screening a human genome DNA library using a probe synthesized based on the sequences disclosed in the databases or the sequences represented by SEQ ID Nos.1 to 200. Also by method of PCR using a human genome library as a template, individual genes (DNA fragments) can be obtained. Even in the case of an unknown register number, the RNA of an intended gene can be identified by using a microarray GeneChip U133 probe set shown in Column A, and a library screening using this RNA or cDNA as a probe may for example be employed for obtaining the intended gene.

The resultant gene can be amplified by an ordinary gene amplification method such as PCR, NASBN (nucleic acid sequence-based amplification), TMA (transcription-mediated amplification) and SDA (strand displacement amplification) to obtain purified DNA fragment.

According to an inventive housekeeping gene, its transcription product can be used as a reference in comparing respective data for example when measuring an expressed gene in a specific tissue under two or more different conditions. The transcription product of the housekeeping gene can be used also as a control in investigating the expression localization of any gene.

While a single housekeeping gene can be used, a set of two or more can also be used. As used herein, the term “set” means multiple genes arbitrarily selected from genes Nos.2 to 1189, and a population for example of 5, 10, 25, 50, 100, 150, 200, 500 or 1000 genes can be used. In this context, it is preferable to select the genes predominantly from the smaller number-designated genes of FIGS. 1 to 39. Thus, 1189 genes in FIGS. 1 to 39 are listed in an order from smaller CV values (smaller difference in expression frequency between respective tissues). Accordingly, the gene list in FIGS. 1 to 30 provides a number of genes having a higher expression similarity in multiple tissue comparison to β-actin (No.176) and glycerardehyde-3-phosphate dehydrogenase (No.182) that are employed frequently as controls in investigating the expression localization of any gene.

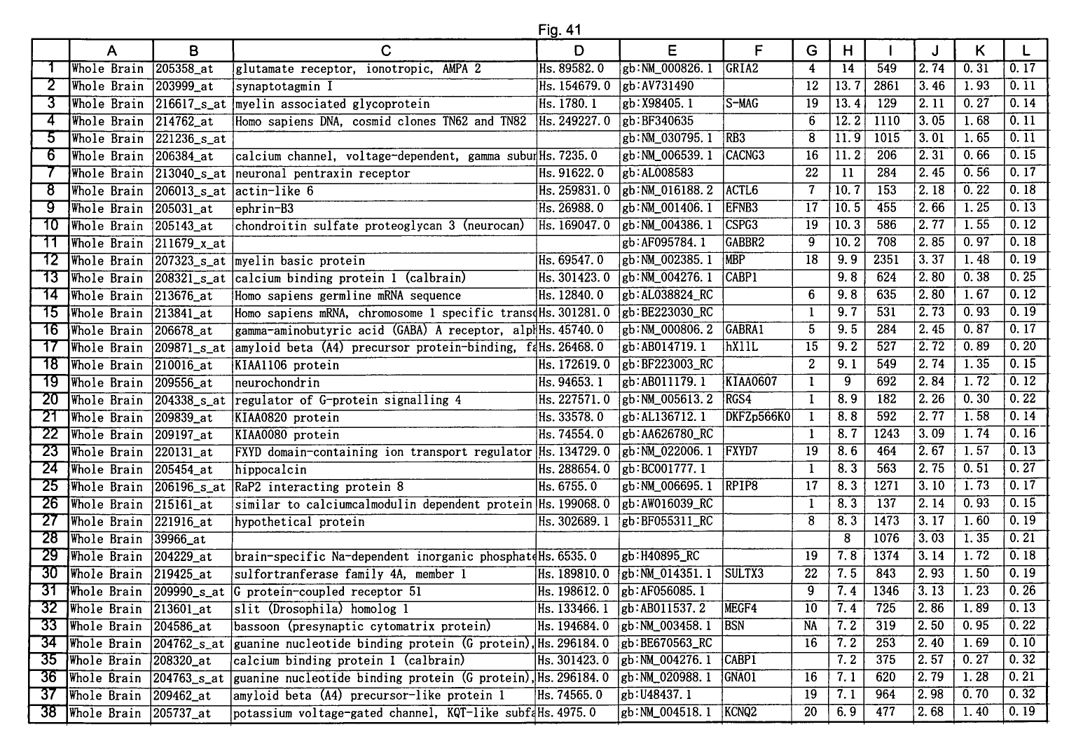

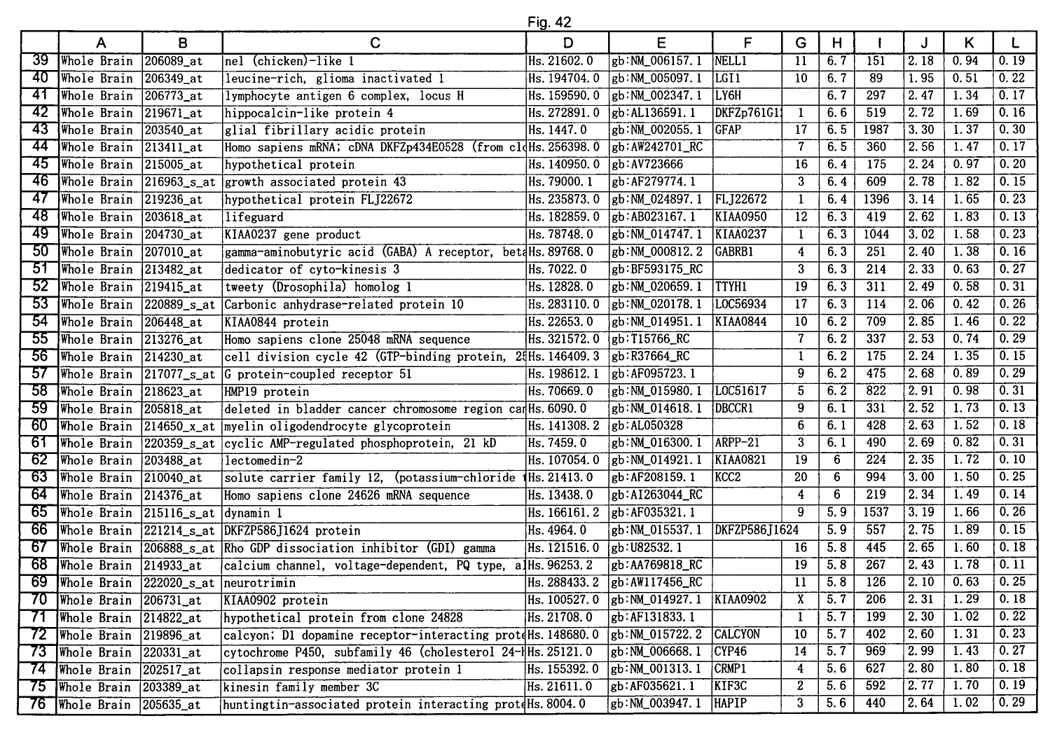

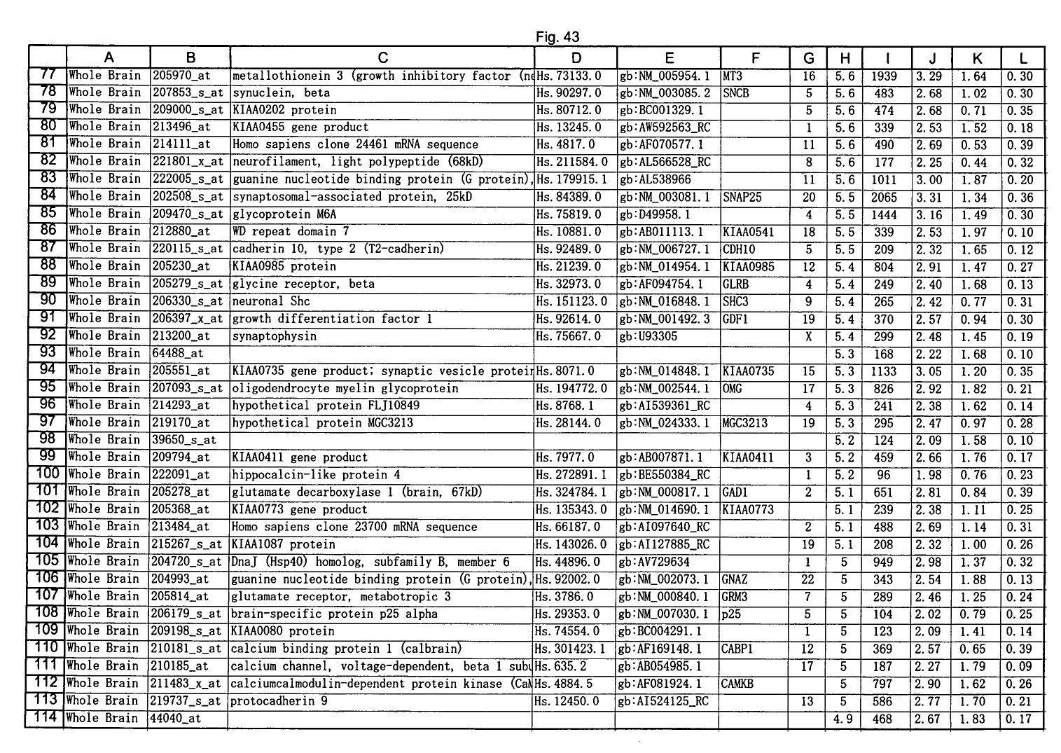

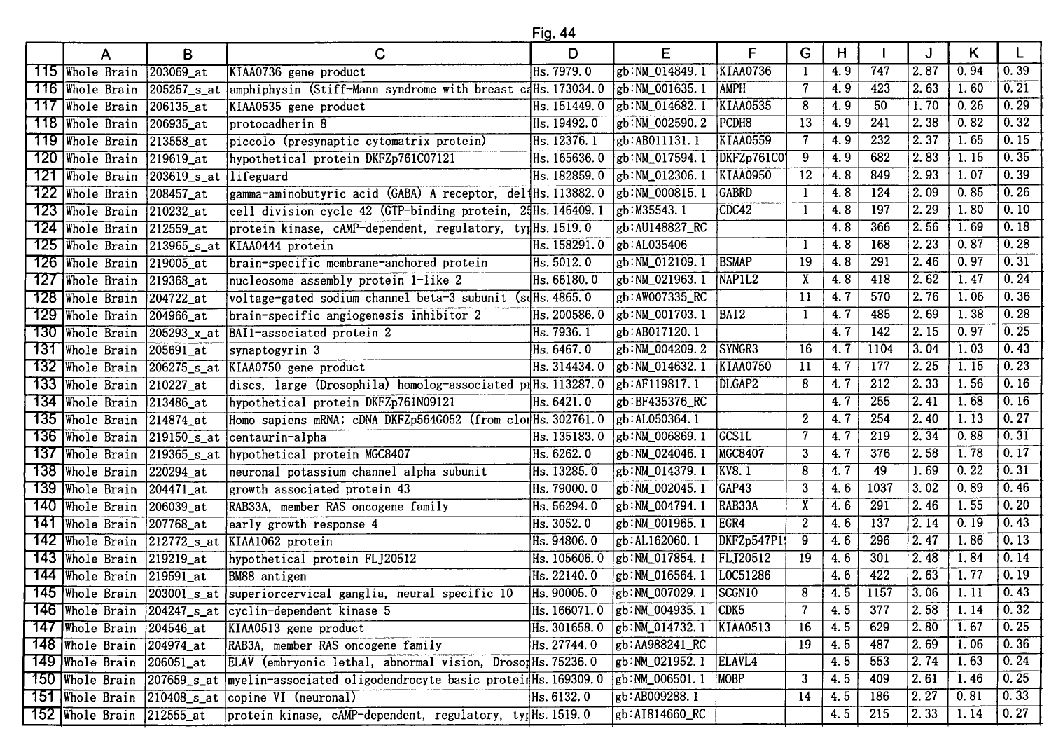

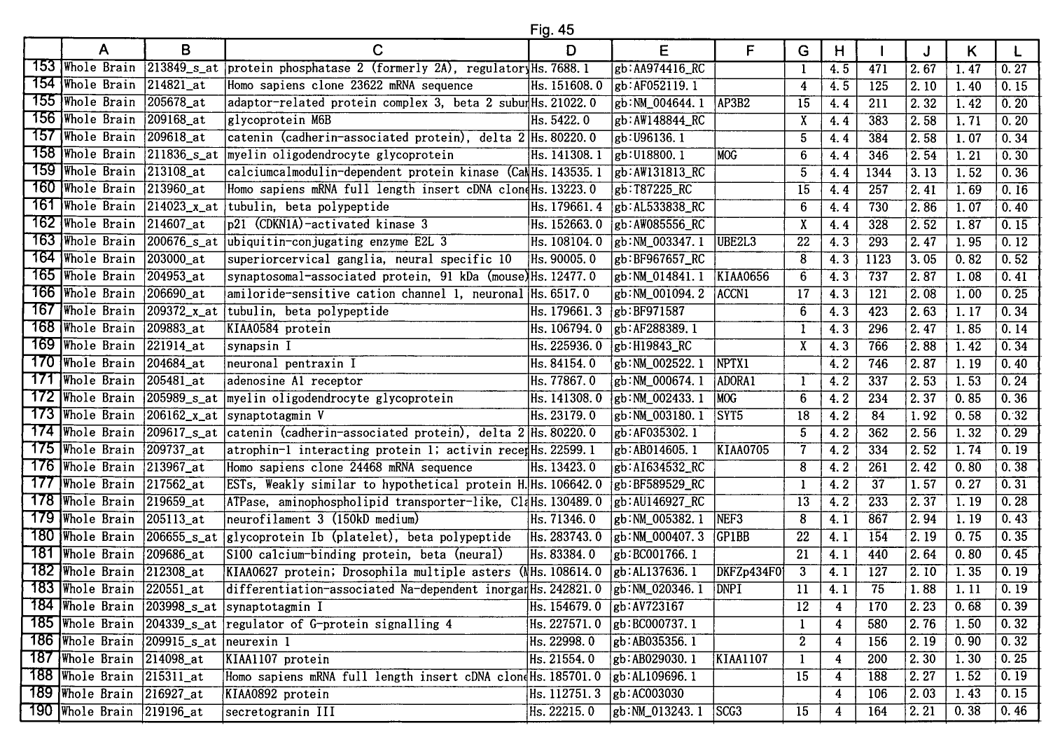

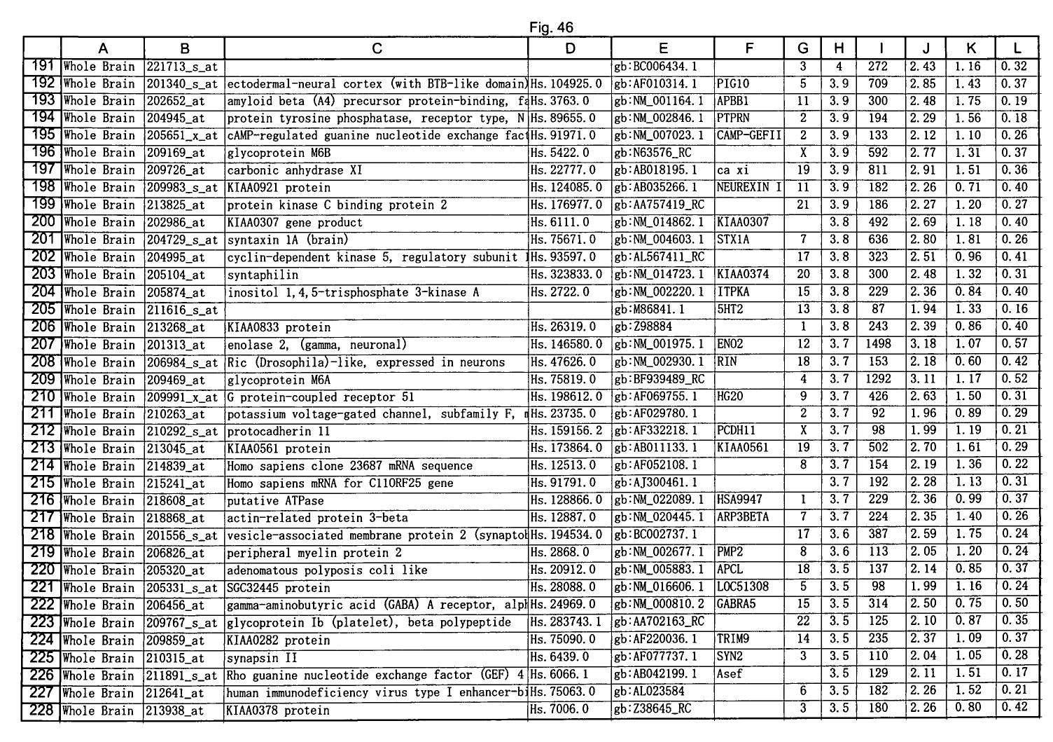

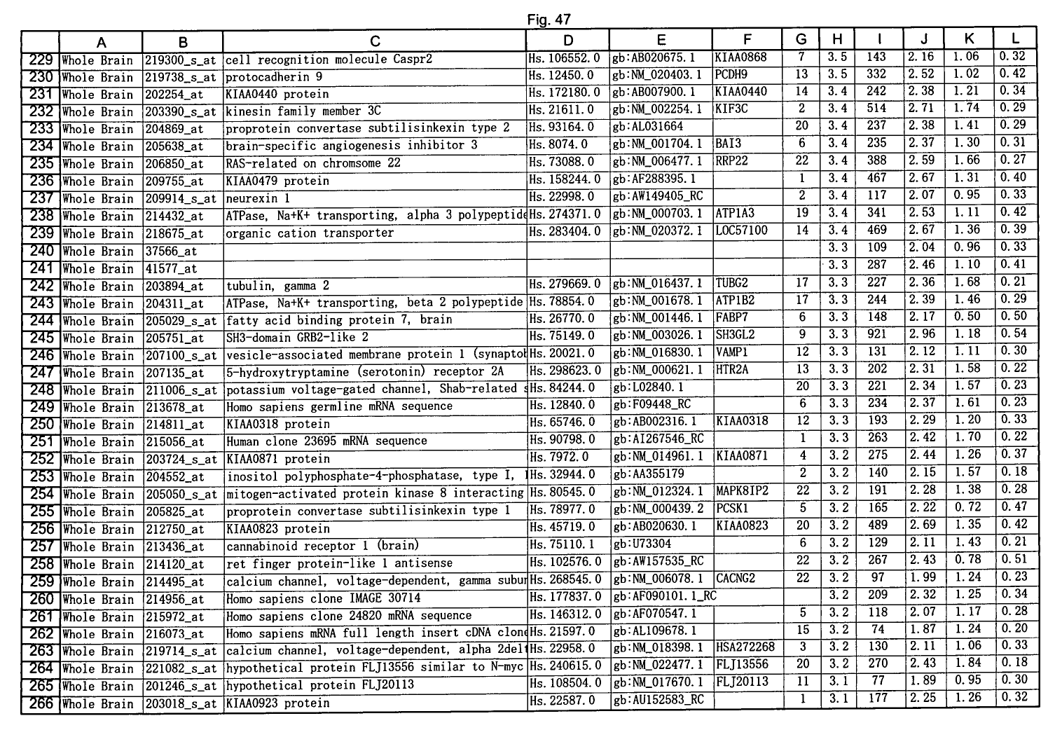

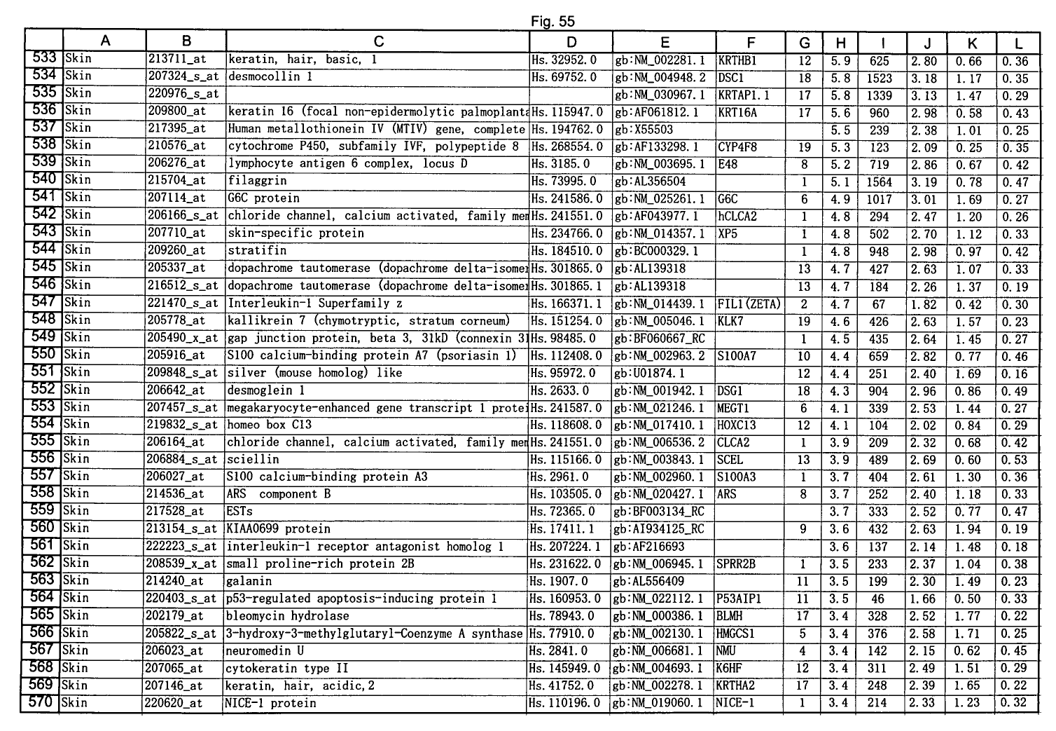

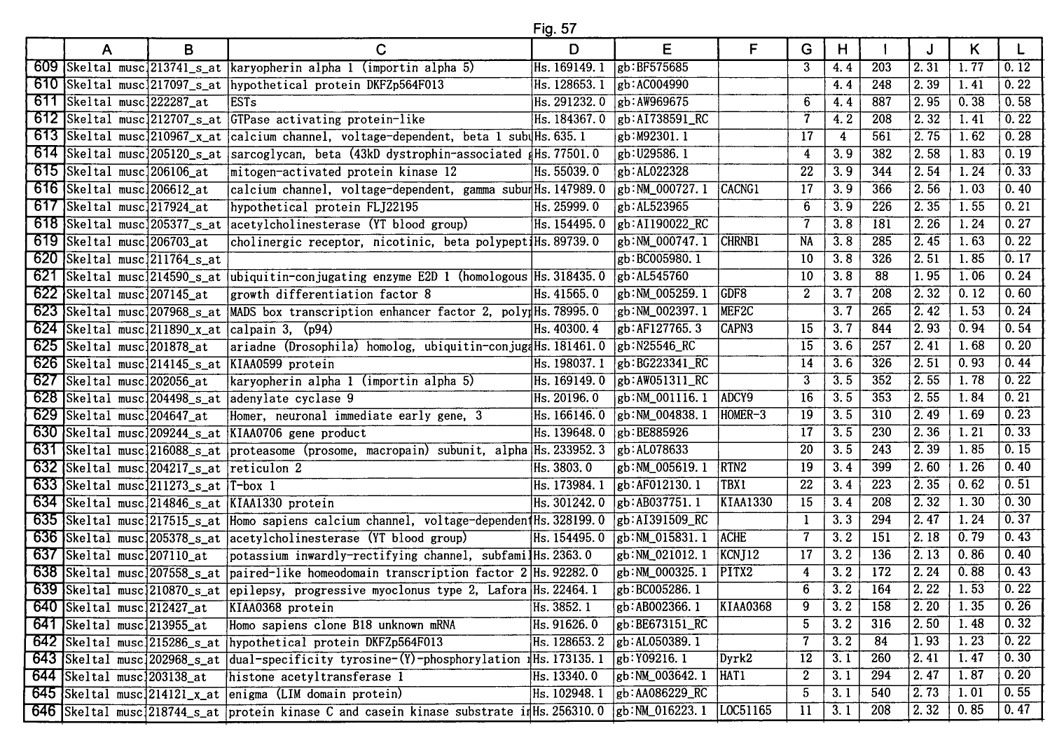

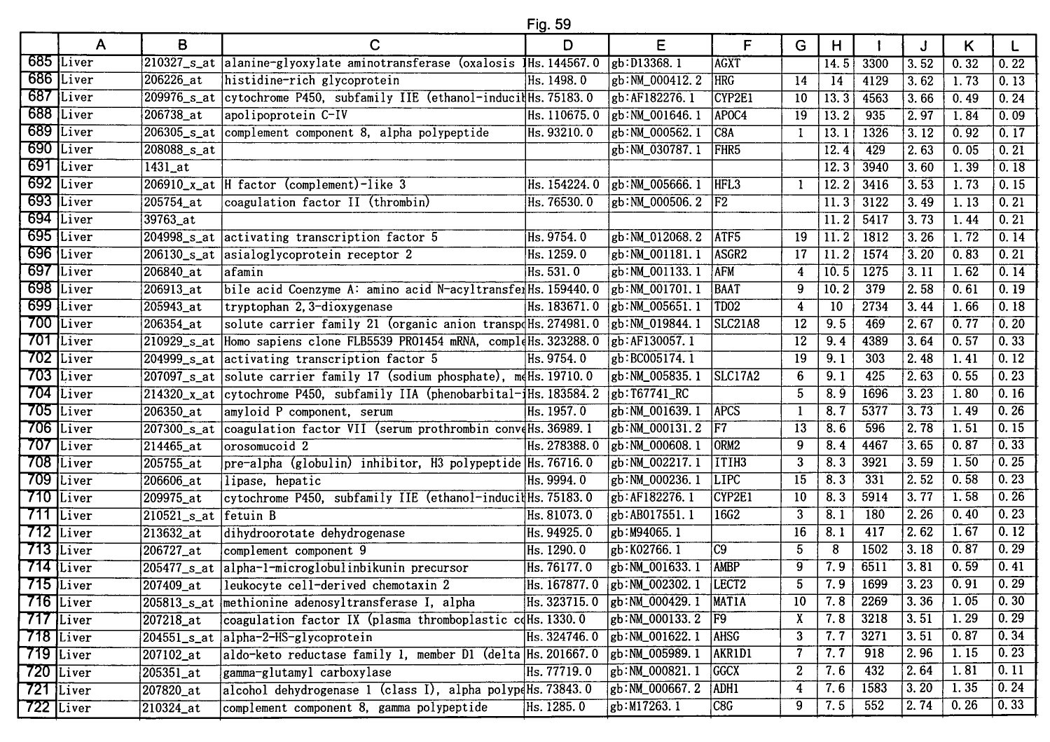

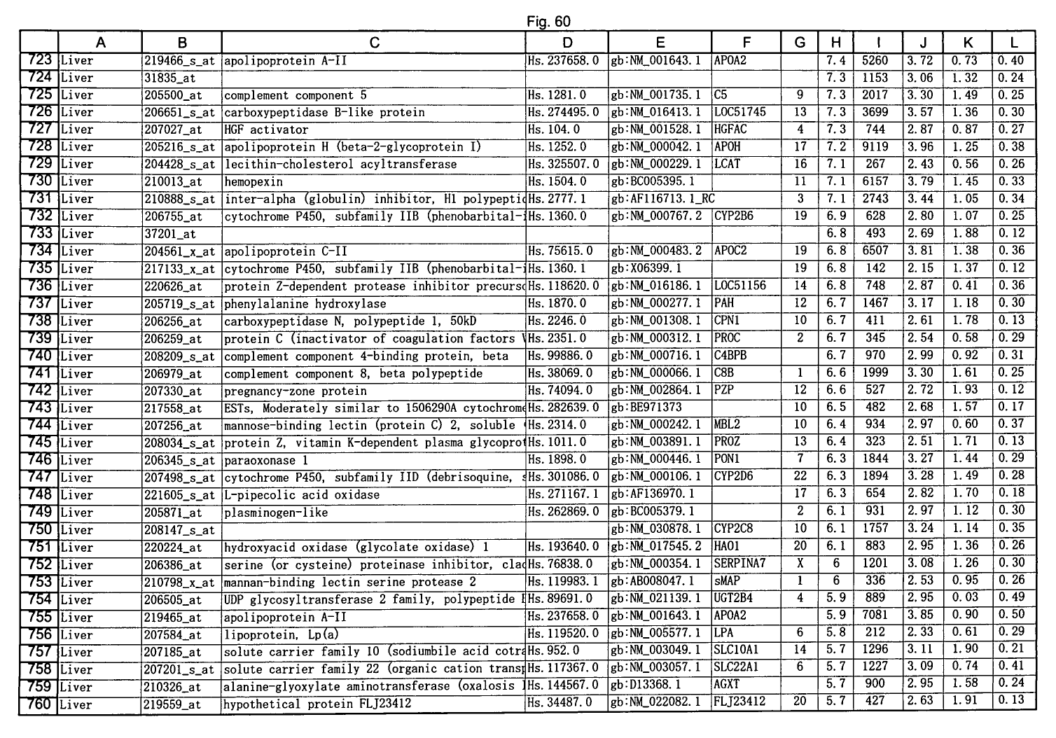

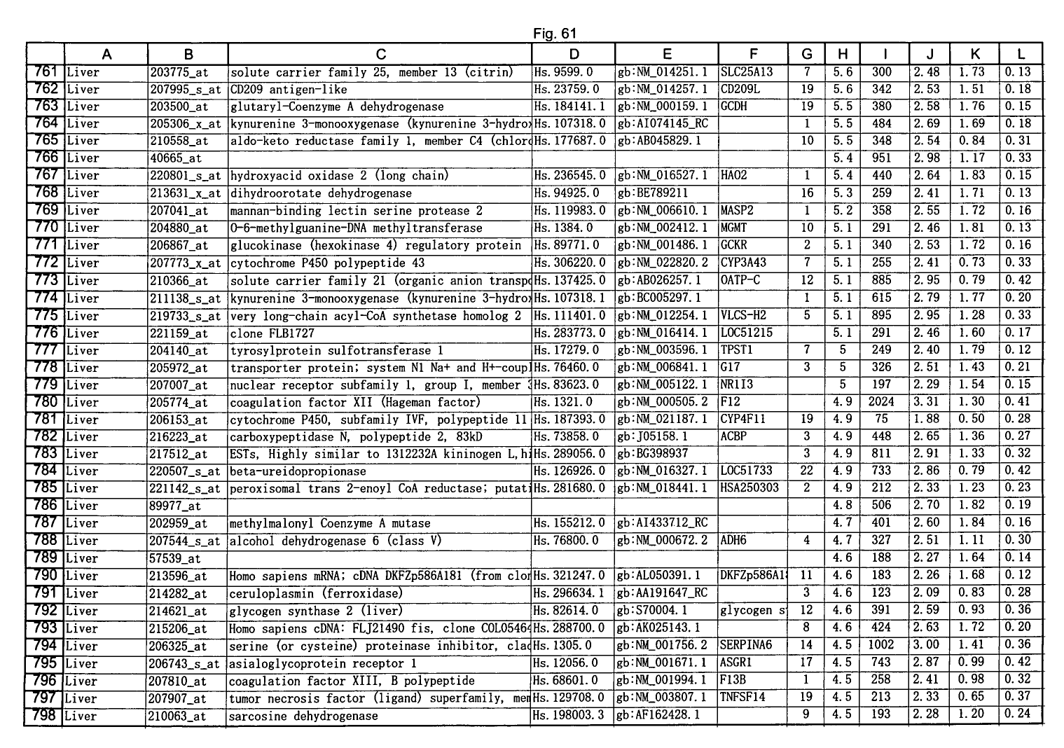

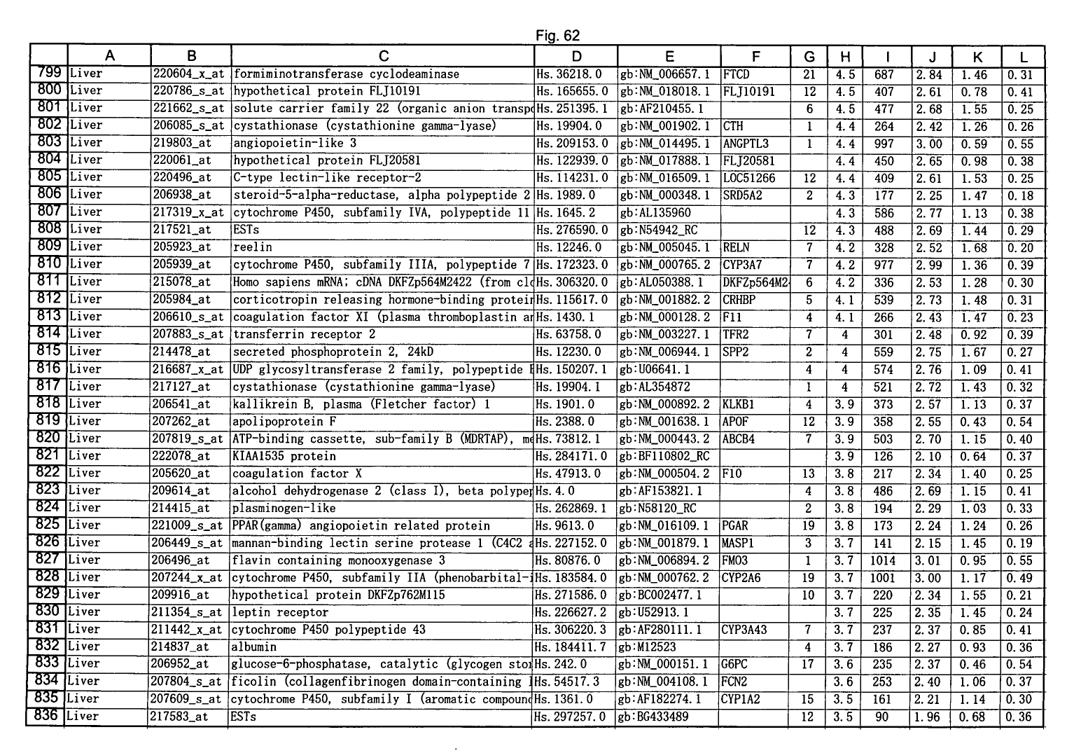

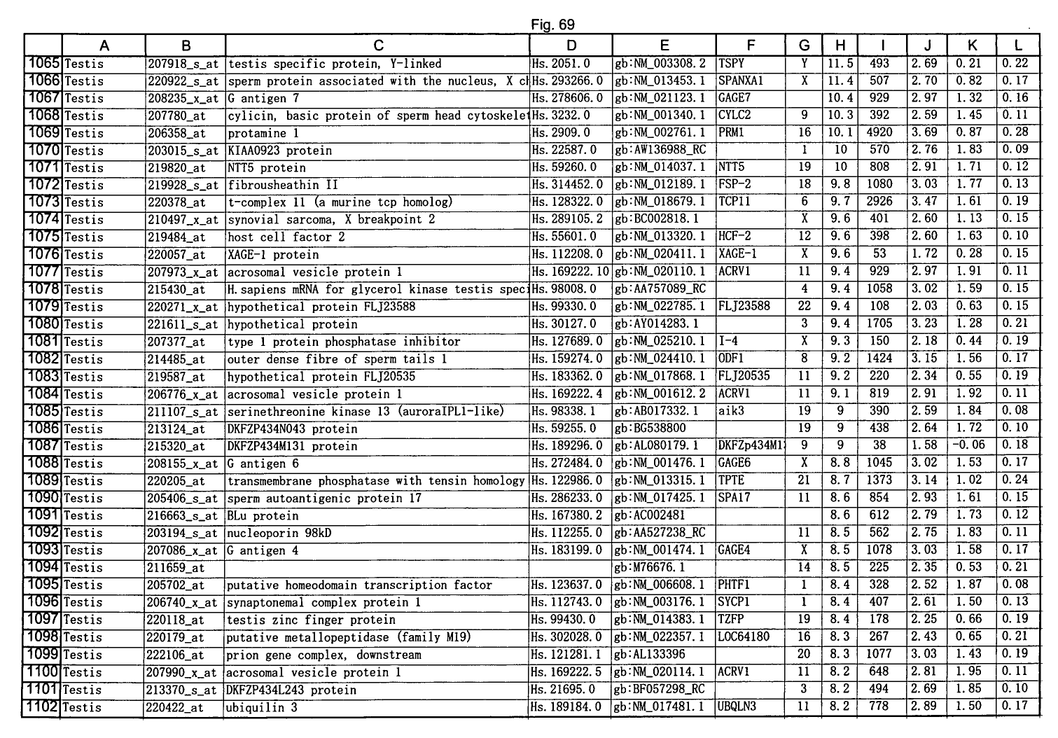

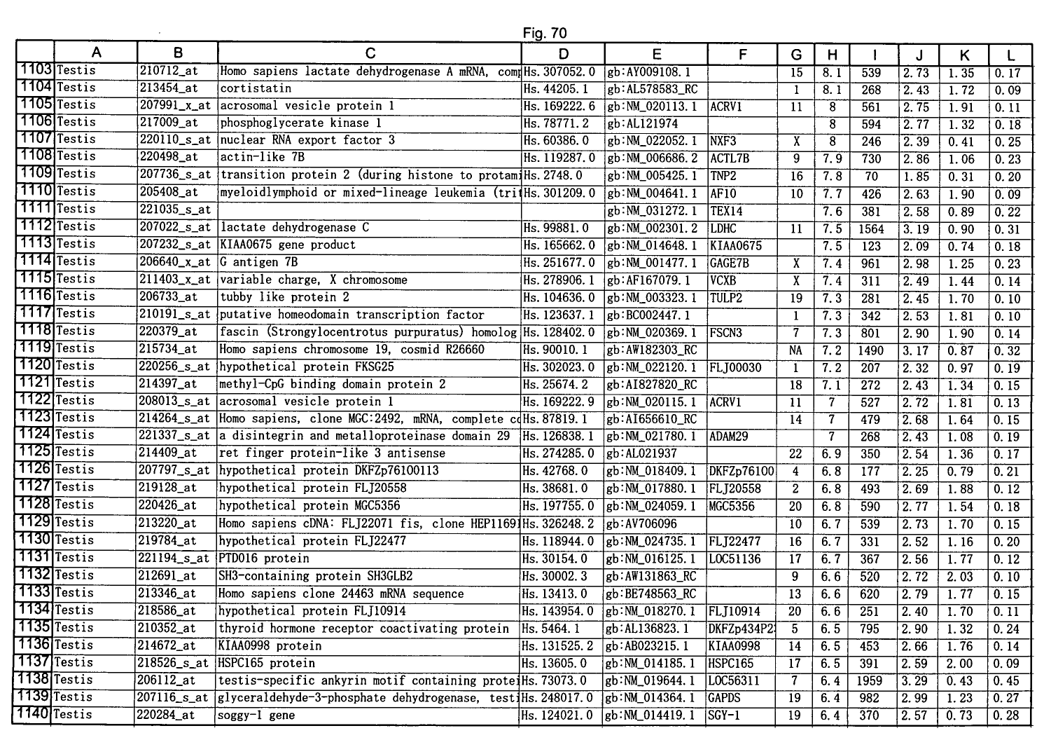

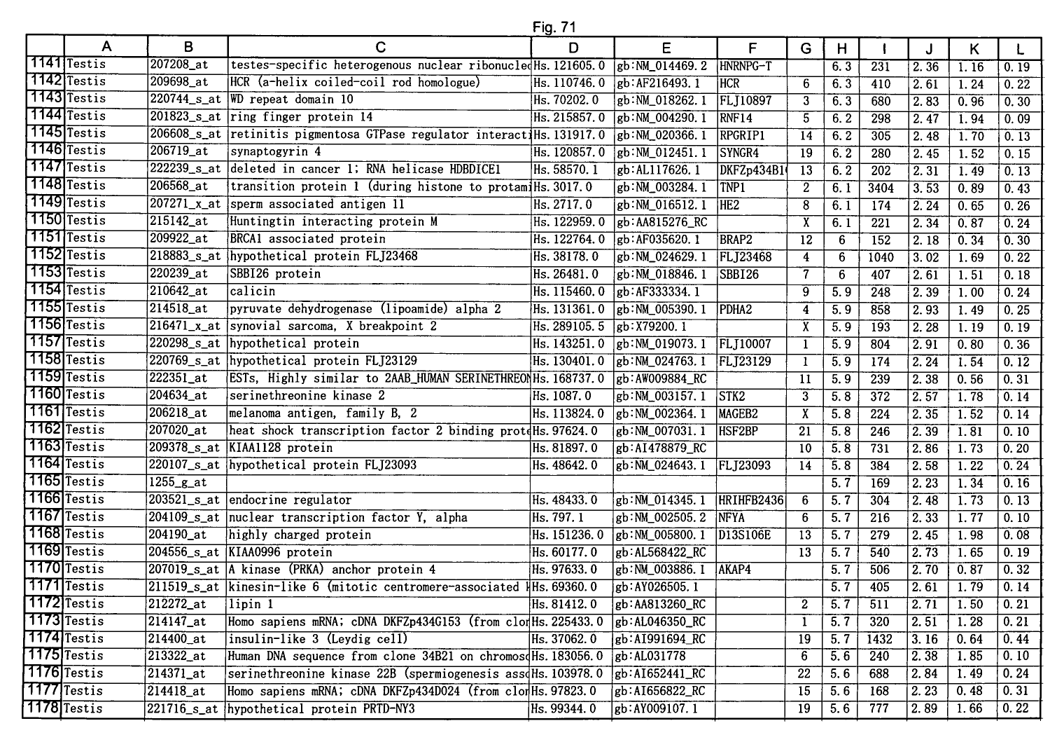

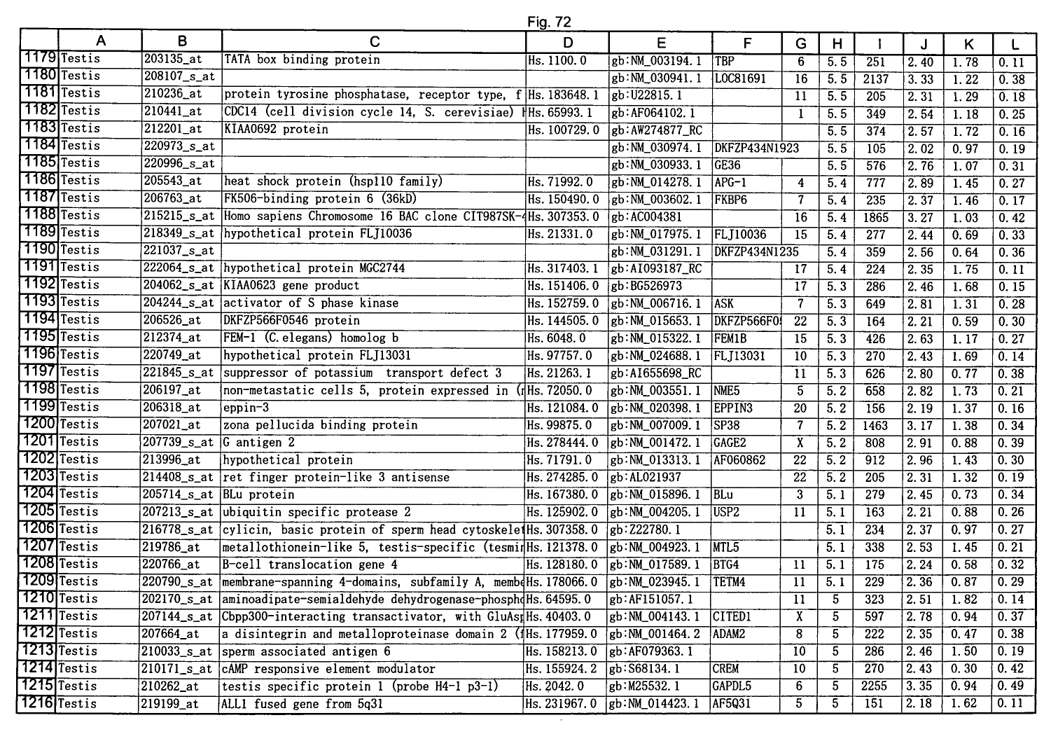

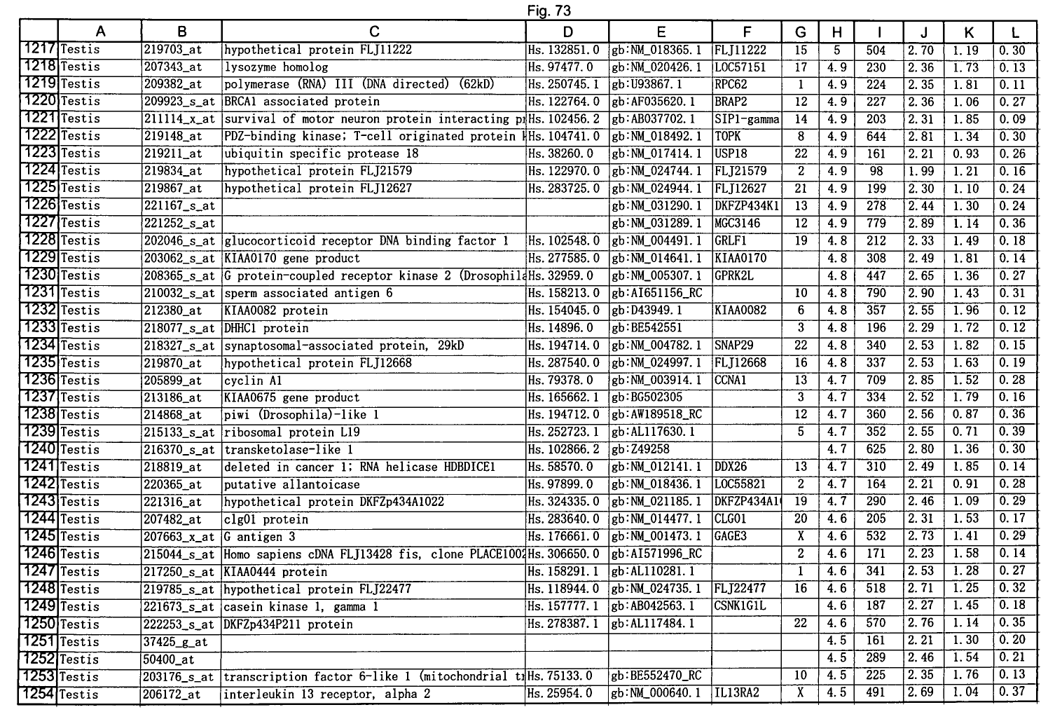

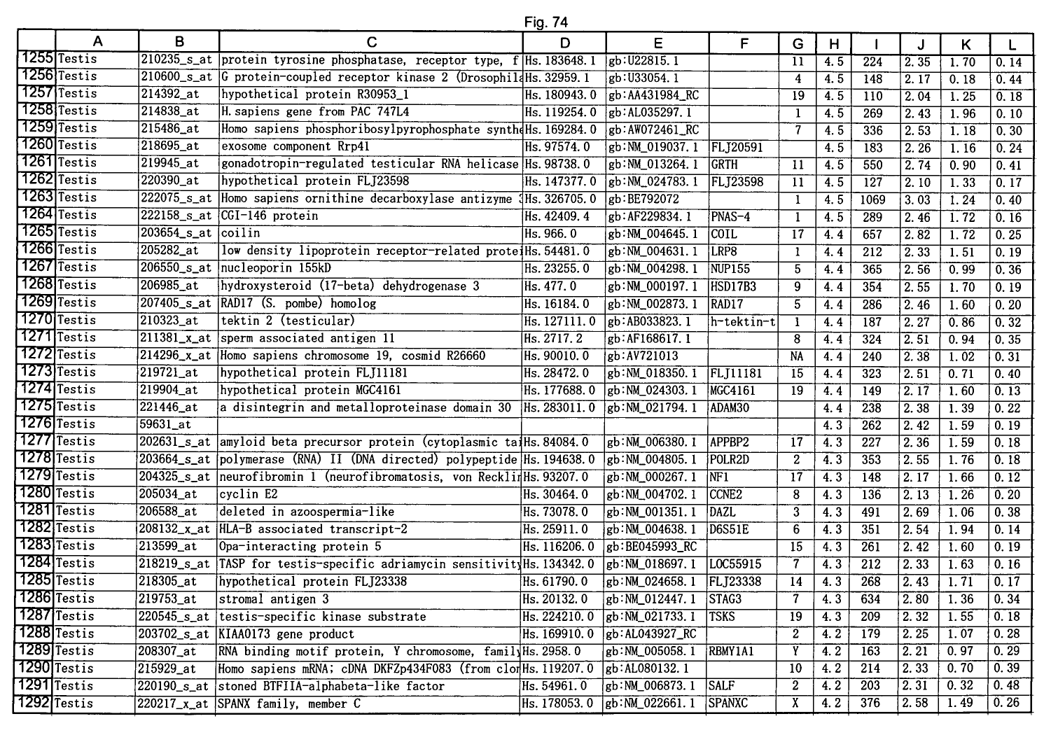

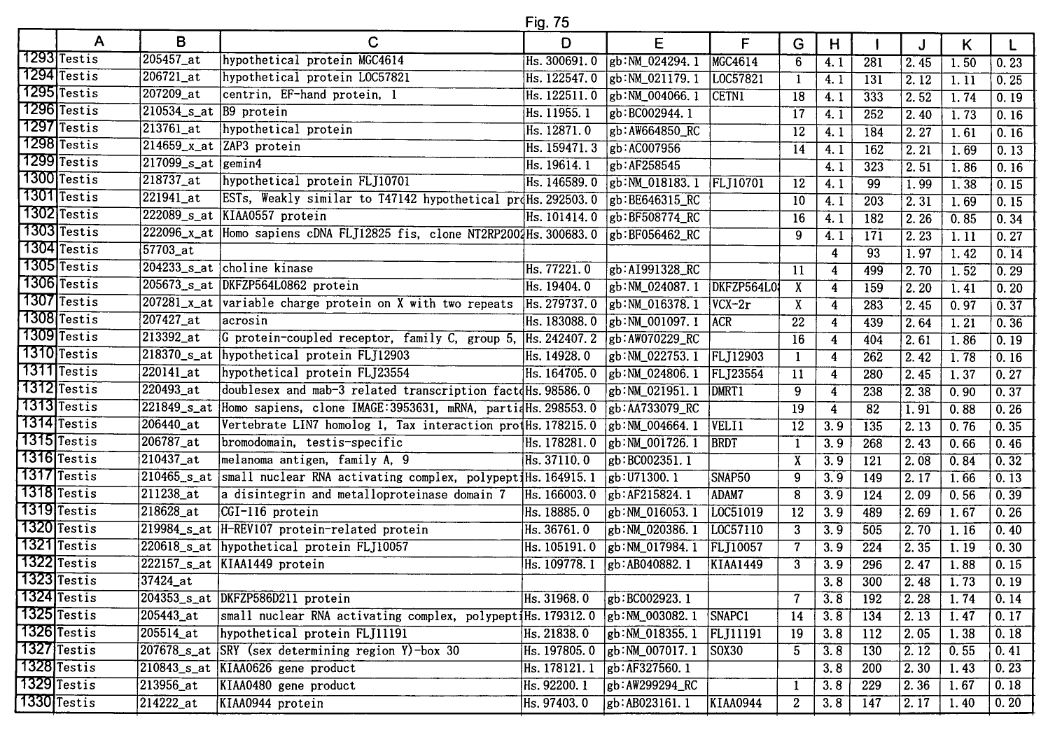

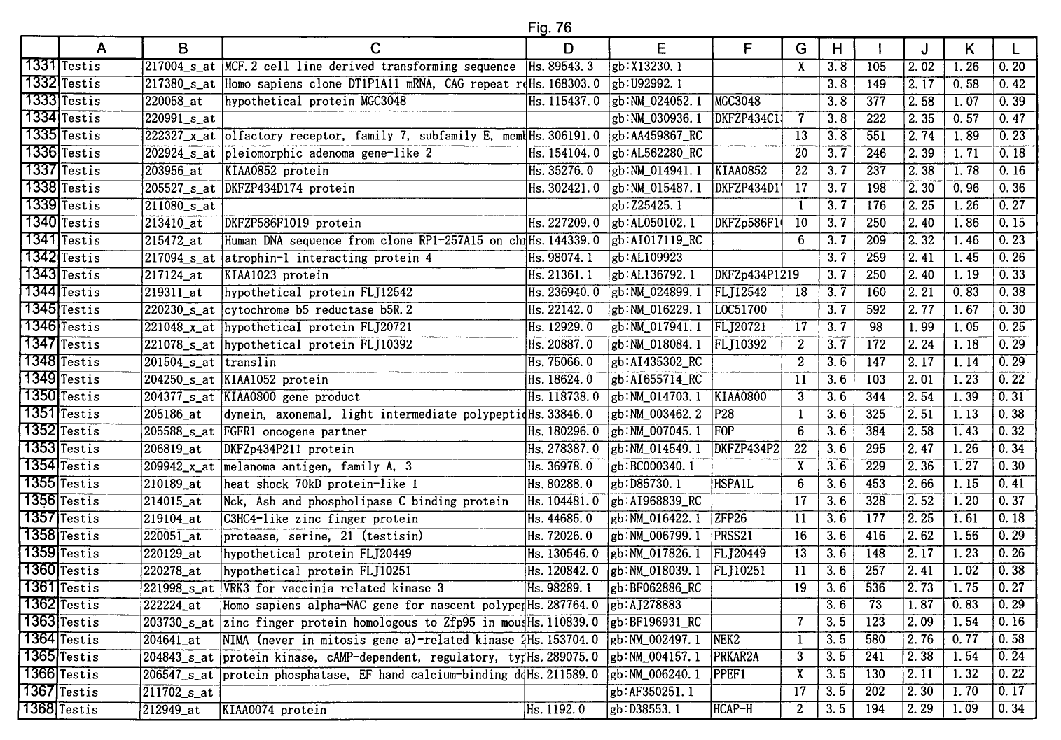

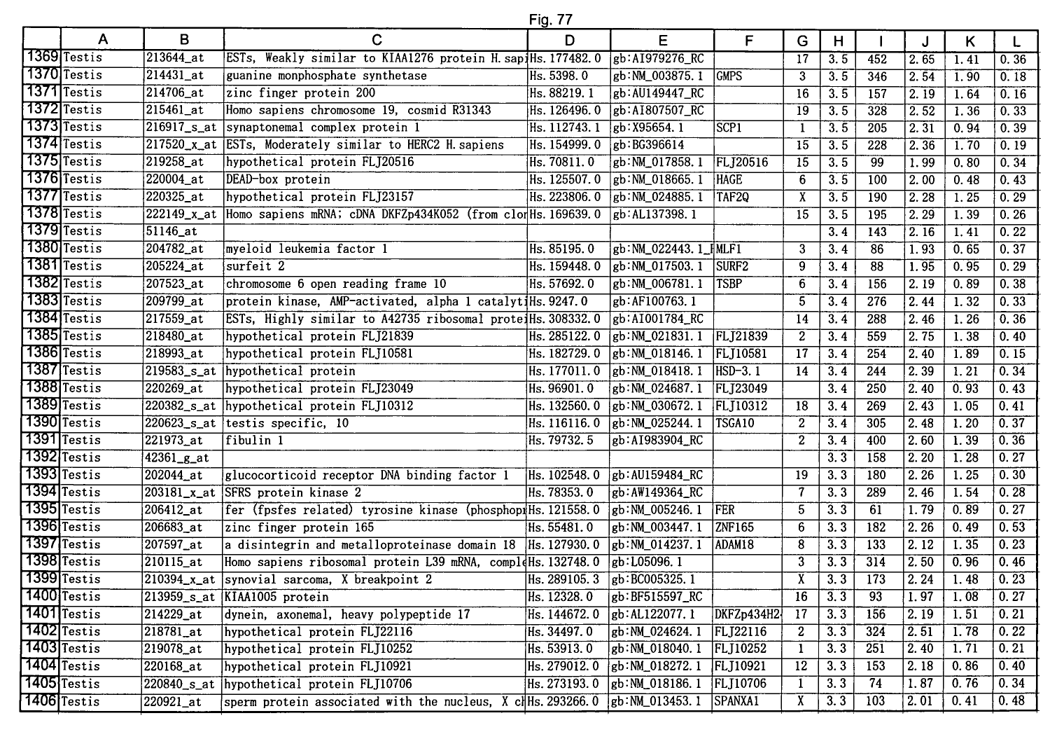

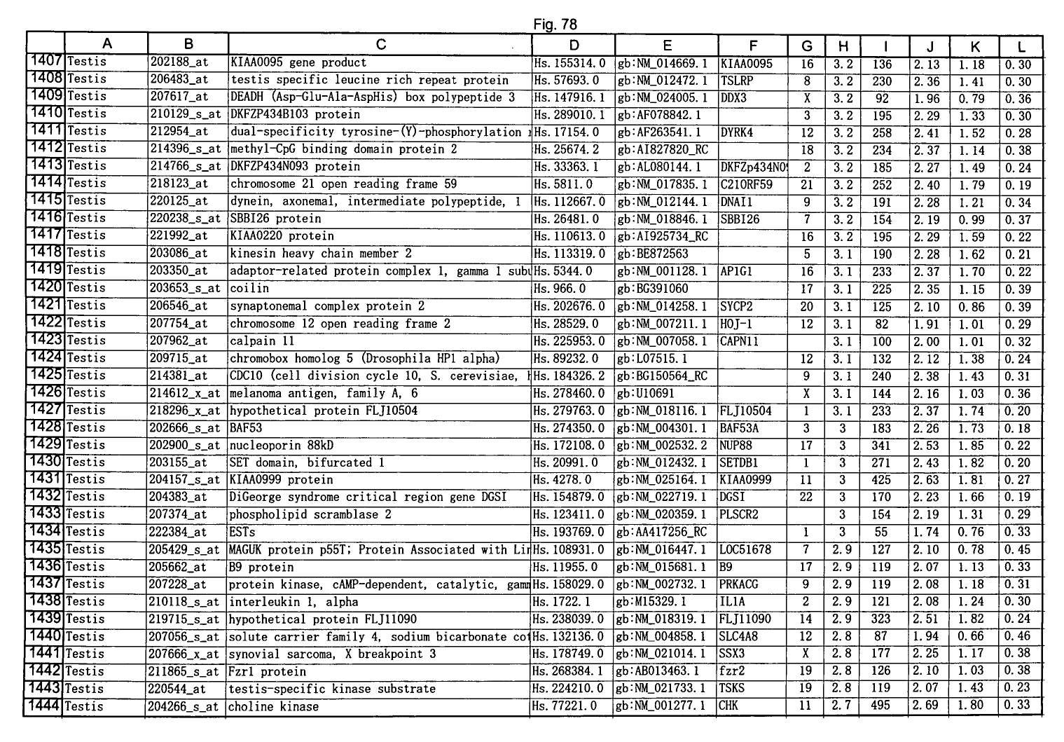

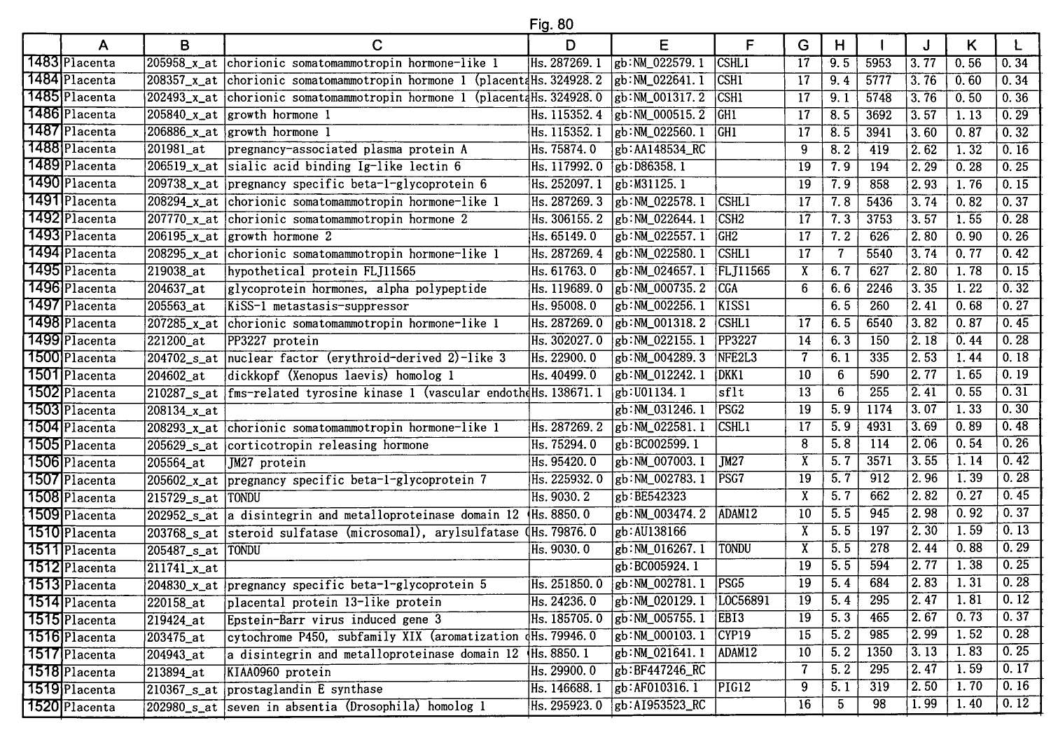

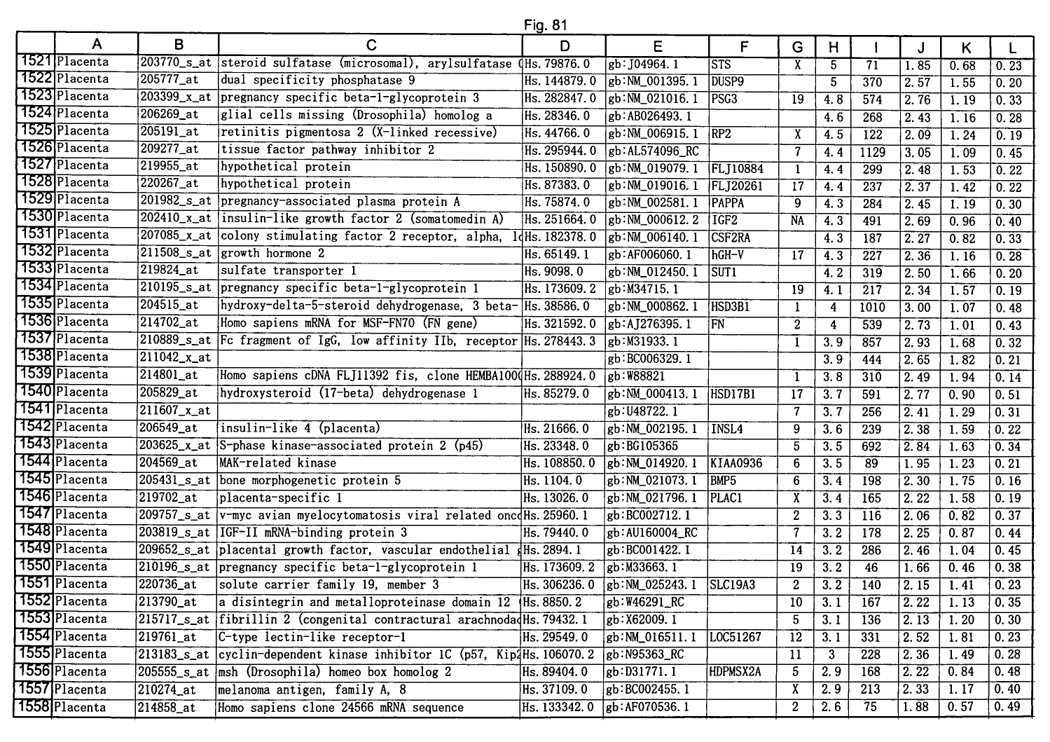

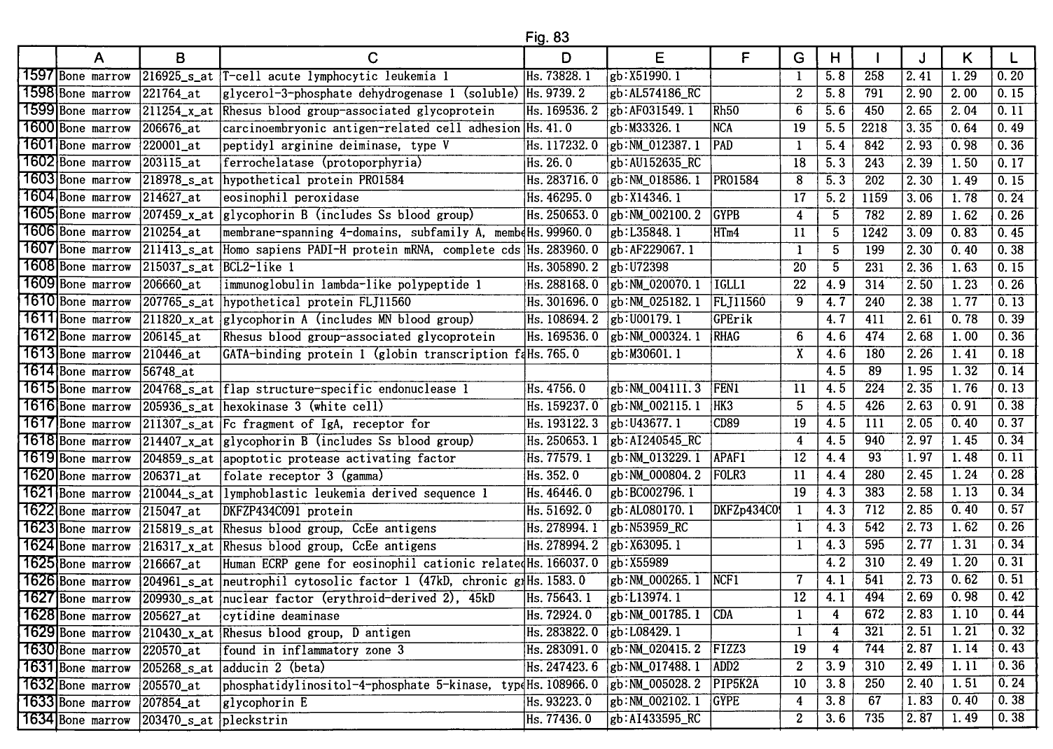

Also in the case of the tissue-specific genes of the invention, individual gene DNA fragments can be obtained based on the probe sets shown in Column B in FIGS. 41 to 85 and the GeneBank register numbers shown in Column E, and then amplified and purified.

These tissue-specific genes or a set thereof can be used in an examination for the differentiation from an undifferentiated cell (ES cell or stem cell) to a specific cell using the presence of a transcription product or the degree of gene expression as an index.

An inventive transcription product is an RNA molecule of each of the above-mentioned housekeeping genes and tissue-specific genes, a protein or peptide translated and expressed from this RNA molecule, or a cDNA fragment synthesized artificially from the RNA molecule. The RNA molecule or cDNA can be obtained based for example on a known method (for example see Mol. Cell Biol. 2, 161-170, 1982; J. Gene 25, 263-269, 1983: Gene, 150, 243-250, 1994). The protein or peptide can be obtained by a gene engineering method using the before-mentioned gene or cDNA fragment (for example, a method employing an in vitro transcription system or host-vector system). For example in a method employing a host/vector system, a transformant cell is cultured to obtain a cell culture, which is then subjected to an isolation and purification by means of a treatment with a denaturing agent such as urea, or with a surfactant, ultrasonic treatment, enzyme digestion, salting-out or solvent precipitation, dialysis, centrifugation, ultrafiltration, gel-filtration, SDS-PAGE, isoelectric electrophoresis, ion exchange chromatography, hydrophobic chromatography, affinity chromatography, reverse-phase chromatography and the like to obtain an intended protein and the like. A protein fused with glutathion-S-transferase (GST) or green fluorescent protein (GFP) or a protein having a His tag or FLAG tag added thereto may also be included in the inventive protein and the like.

Any of these transcription products can be used for a target or means for determining the expression level of the housekeeping gene or the tissue-specific gene described above. For example, RNA can be examined for its expression by means of a northern blotting, slot blotting, dot blotting, DNA microarray and the like. A protein can be measured by method of sandwich assay, ELISA, immunoprecipitation, western blotting and the like, in which a specific antibody for the protein and the like is employed. A cDNA fragment can be employed as a capture probe for DNA microarray.

An inventive oligonucleotide probe is a nucleotide (RNA or DNA) sequence which hybridizes with the above-mentioned housekeeping gene DNA or tissue-specific gene DNA, or transcription product thereof which is an RNA molecule or cDNA fragment. The probe may be deposited on a soluble or insoluble polymer, or may be deposited on or bound to a solid support such as a filter, sheet, chip, slide, bead and the like. Alternatively, it may be labeled with an enzyme, fluorescent dye, radioisotope and the like. Any of such probes can be used in a known hybridization assay using a DNA microarray and the like. The assay can be conducted as desired for its purpose under various stringent conditions in the steps of hybridization and washing (modification of salt concentration, organic solvent (formamide and the like) concentration, temperature) (for example, see U.S. Pat. No. 6,100,037 and the like).

An inventive DNA microarray is fitted with a set of the above-mentioned respective transcription products (cDNA fragments) or oligonucleotide probes as a capture probe. The capture probe may be in a mode in which its base is synthesized on a base-by-base basis on a support (Affymetrix type), or in a mode in which a probe DNA fragment is spotted on a support (Stamford type) (for example, see U.S. Pat. No. 5,474,796, Schena, M. et al., Proc. Natl. Acad. Sci. 93:10614-10619, 1996; PCT WO95/251116; PCT WO95/35505; Heller, R. A. et al., Proc. Natl. Acad. Sci. 94:2150-2155, 1997; U.S. Pat. No. 5,605,662 and the like).

The identification of the inventive housekeeping genes and tissue-specific genes are further detailed below.

1. Samples

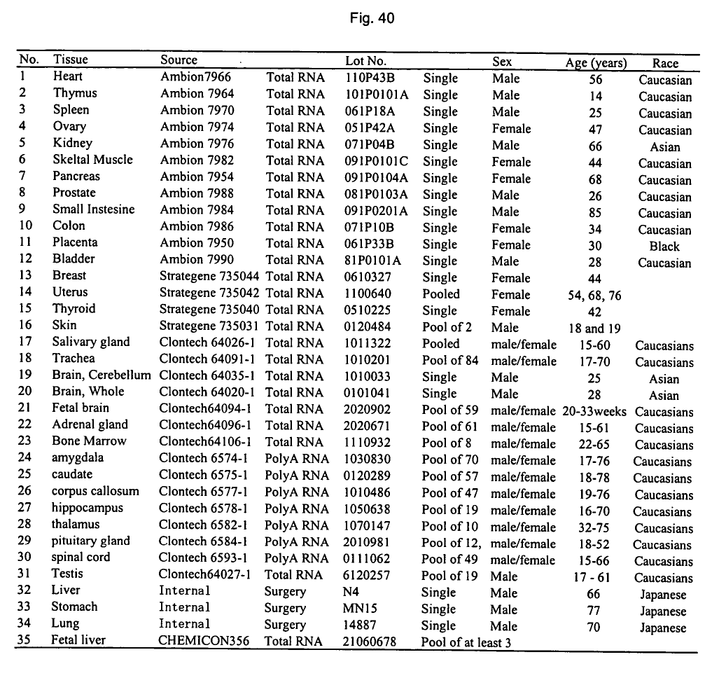

The RNA samples derived from 35 tissues shown in FIG. 40 were employed. A total RNA and a polyA RNA were purchased from Clontech (Palo Alto, Calif.), Ambion (Austin, Tex.) and Stratagene (La Jolla, Calif.). Each liver, stomach or lung was obtained from a surgical resection sample after obtaining an informed consent, and was subjected to conventional method to obtain RNA.

2. DNA Microarray Protocol

The microarray experiment procedure was in accordance with the GeneChip expression analysis technical manual by Affymetrix. Briefly, each 10 mg of the RNA was subjected to synthesize a biotinylated cDNA. This cDNA was hybridized with an oligonucleotide array (GeneChip Human U133 array: Affymetrix, Santa Crara, Calif.). After washing, the array was stained with a streptoavidin-phycoerythrin and its image data were accumulated using a Hewlett-Packard Scanner and then analyzed. The “average difference” of each gene probe on the array was calculated by an analysis program “GeneChip Analysis Suite software version 5.0”. The average difference was normalized so that the median of each array became 100. In addition to the average difference value, the detection value P of each gene was calculated in accordance with the density difference between the match-probe and the mismatch-probe of several ten combinations or more.

3. Statistic Analysis

As a standard for the gene expression, the detection value P less than 0.05 was employed, and a gene whose detection value P was less than 0.05 for all of the 35 tissues was regarded as a housekeeping gene. Similarly, a gene whose expression detection value P in a single tissue was less than 0.05 and whose expression detection values P in other 34 tissues were 0.05 or more was regarded as a tissue-specific gene.

A housekeeping gene was subjected to a calculation for a coefficient of variation (CV) as reported before (Hsiao L. L. et al., Physiol. Genomics 7:97-104, 2001).

In the identification of a tissue-specific gene, a clustering analysis was conducted for the purpose of obtaining an overall similarity among different tissues. A stratified clustering was conducted by an operation of the “Cluster” program and the “Treeview” program (Eisen. M. B. et al., Proc. Natl. Acad. Sci. USA 95:14863-14868, 1998). The distance metrics is of a piasson correlation. Prior to the clustering, genes whose standard deviation between samples was 50 or less or whose difference between the maximum expression and the minimum expression in each tissue was 200 or less was excluded. Thereafter, the data were converted logarithmically and standardized by means of making the mean value and deviation of a gene expression identical to each other. Finally, an average linkage clustering was obtained using the cluster programs.

### INDUSTRIAL APPLICABILITY

As detailed above, the present invention provides a novel population of housekeeping genes expressed commonly over 35 different human normal tissues, and a novel population of tissue-specific genes expressed specifically in a single tissue. A gene or a gene set selected from these gene populations is useful as a reference and the like for a normal gene expression or disease-associated gene expression measured under various conditions. A transcription product from the gene or a probe for the gene is also useful as a DNA microarray capture probe.

1. A human housekeeping expressed commonly over 35 different human tissues, which gene is selected from the group consisting of genes Nos.1 to 1189 on FIGS. 1 to 39.

2. The gene of claim 1, which is selected from genes Nos.1 to 200 on FIGS. 1 to 7.

3. A transcription product of the gene of claim 1.

4. The transcription product of claim 3, which is an RNA or cDNA.

5. The transcription product of claim 4, wherein the cDNA has the base sequence of any of SEQ ID NOs: 1, 3, 5, 6, 8, 10-12, 14, 16-19, 21, 23, 25, 27, 28, 30-35, 37-39, 41-43, 45, 47, 49, 51, 53, 54, 56, 57, 59, 60, 62, 64, 66, 67, 69, 71, 72, 74, 75, 77, 79, 80, 82, 84, 86, 87, 89, 91, 93, 95, 97, 98, 100, 102, 103, 105, 107, 109, 111, 113, 115, 117, 118, 120, 122, 123, 125, 127, 128, 130-132, 134, 136, 137, 139, 141-143, 145, 146, 148, 150, 152, 154, 156, 157, 159, 161, 163, 164, 166, 168-170, 172, 174, 176, 178, 180, 181, 183, 185, 186, 188, 189, 191, 193, 195, 196, 198, 199, 201, 202, 204, 206-208, 210, 212, 214, 216, 218, 219, 221, 222, 224, 225, 227, 229, 231, 233, 234, 236, 237, 239, 241, 243, 245, 247, 248, 250, 252, 254, 256, 258, 260, 262, 263, 265, 267, 269, 270, 272, 274, 276-278, 280, 281, 283, 285-289, 291, 293, 295, 297, 299, 300, 302, 304, 306, 307, 309, 311-313, 315, 317, 319, 320, 322, 324, 326, 328, 330 and 331.

6. An oligonucleotide probe, which hybridizes with the gene of claim 1.

7. The probe of claim 6, wherein the transcription product is a cDNA having the base sequence of any of SEQ ID NOs: 1, 3, 5, 6, 8, 10-12, 14, 16-19, 21, 23, 25, 27, 28, 30-35, 37-39, 41-43, 45, 47, 49, 51, 53, 54, 56, 57, 59, 60, 62, 64, 66, 67, 69, 71, 72, 74, 75, 77, 79, 80, 82, 84, 86, 87, 89, 91, 93, 95, 97, 98, 100, 102, 103, 105, 107, 109, 111, 113, 115, 117, 118, 120, 122, 123, 125, 127, 128, 130-132, 134, 136, 137, 139, 141-143, 145, 146, 148, 150, 152, 154, 156, 157, 159, 161, 163, 164, 166, 168-170, 172, 174, 176, 178, 180, 181, 183, 185, 186, 188, 189, 191, 193, 195, 196, 198, 199, 201, 202, 204, 206-208, 210, 212, 214, 216, 218, 219, 221, 222, 224, 225, 227, 229, 231, 233, 234, 236, 237, 239, 241, 243, 245, 247, 248, 250, 252, 254, 256, 258, 260, 262, 263, 265, 267, 269, 270, 272, 274, 276-278, 280, 281, 283, 285-289, 291, 293, 295, 297, 299, 300, 302, 304, 306, 307, 309, 311-313, 315, 317, 319, 320, 322, 324, 326, 328, 330 and 331.

8. A set of at least two housekeeping genes expressed commonly over 35 different human tissues, wherein each gene of the set is selected from the group consisting of genes Nos.1 to 1189 on FIGS. 1 to 39.

9. The set of genes of claim 8, wherein each gene is selected predominantly from the gene No.1 on FIGS. 1 to 39.

10. The set of genes of claim 8, wherein each gene is selected from genes Nos.1 to 200 on FIGS. 1 to 7.

11. A set of transcription products from each gene of the set of the genes of claim 8.

12. The set of transcription products of claim 11, wherein the product is an RNA or cDNA.

13. The set of transcription products of claim 12, wherein the cDNA has the base sequence of any of SEQ ID NOs:1, 3, 5, 6, 8, 10-12, 14, 16-19, 21, 23, 25, 27, 28, 30-35, 37-39, 41-43, 45, 47, 49, 51, 53, 54, 56, 57, 59, 60, 62, 64, 66, 67, 69, 71, 72, 74, 75, 77, 79, 80, 82, 84, 86, 87, 89, 91, 93, 95, 97, 98, 100, 102, 103, 105, 107, 109, 111, 113, 115, 117, 118, 120, 122, 123, 125, 127, 128, 130-132, 134, 136, 137, 139, 141-143, 145, 146, 148, 150, 152, 154, 156, 157, 159, 161, 163, 164, 166, 168-170, 172, 174, 176, 178, 180, 181, 183, 185, 186, 188, 189, 191, 193, 195, 196, 198, 199, 201, 202, 204, 206-208, 210, 212, 214, 216, 218, 219, 221, 222, 224, 225, 227, 229, 231, 233, 234, 236, 237, 239, 241, 243, 245, 247, 248, 250, 252, 254, 256, 258, 260, 262, 263, 265, 267, 269, 270, 272, 274, 276-278, 280, 281, 283, 285-289, 291, 293, 295, 297, 299, 300, 302, 304, 306, 307, 309, 311-313, 315, 317, 319, 320, 322, 324, 326, 328, 330 and 331.

14. A set of oligonucleotide probes, each of which hybridizes with each gene of the set of genes of claim 8.

15. The set probes of claim 14, wherein the transcription product is a cDNA having the base sequence of any of SEQ ID NOs:1, 3, 5, 6, 8, 10-12, 14, 16-19, 21, 23, 25, 27, 28, 30-35, 37-39, 41-43, 45, 47, 49, 51, 53, 54, 56, 57, 59, 60, 62, 64, 66, 67, 69, 71, 72, 74, 75, 77, 79, 80, 82, 84, 86, 87, 89, 91, 93, 95, 97, 98, 100, 102, 103, 105, 107, 109, 111, 113, 115, 117, 118, 120, 122, 123, 125, 127, 128, 130-132, 134, 136, 137, 139, 141-143, 145, 146, 148, 150, 152, 154, 156, 157, 159, 161, 163, 164, 166, 168-170, 172, 174, 176, 178, 180, 181, 183, 185, 186, 188, 189, 191, 193, 195, 196, 198, 199, 201, 202, 204, 206-208, 210, 212, 214, 216, 218, 219, 221, 222, 224, 225, 227, 229, 231, 233, 234, 236, 237, 239, 241, 243, 245, 247, 248, 250, 252, 254, 256, 258, 260, 262, 263, 265, 267, 269, 270, 272, 274, 276-278, 280, 281, 283, 285-289, 291, 293, 295, 297, 299, 300, 302, 304, 306, 307, 309, 311-313, 315, 317, 319, 320, 322, 324, 326, 328, 330 and 331.

16. A DNA microarray carrying the set of transcription products of claim 12.

17. A human whole brain-specific gene selected from the group consisting of genes Nos.1 to 294 on FIGS. 41 to 48, or a set of genes consisting of two or more of human whole brain-specific genes selected from said group.

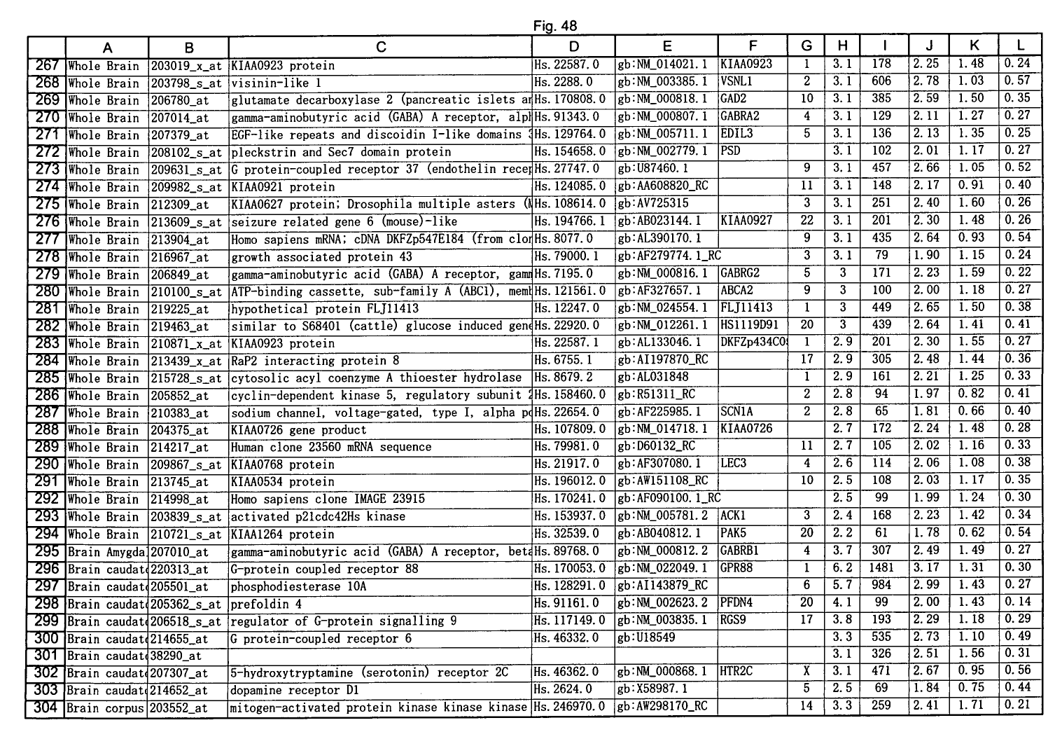

18. A human amygdaloid body-specific gene, which is the gene No.295 on FIG. 48.

19. A human caudate nucleus-specific gene selected from the group consisting of genes Nos.296 to 303 on FIG. 48, or a set of genes consisting of two or more of human caudate nucleus-specific genes selected from said group.

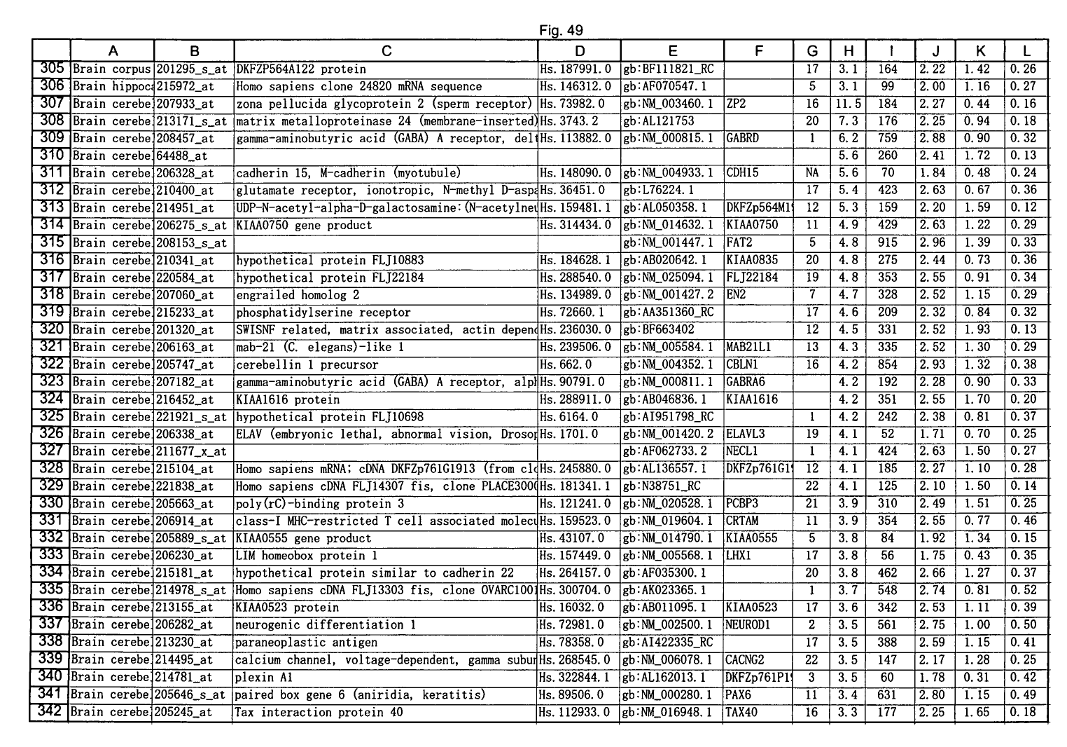

20. A human callosum-specific gene selected from the group consisting of genes Nos.304 to 305 shown on FIGS. 48 to 49, or a set of genes consisting of two or more of human callosum-specific genes selected from said group.

21. A human hippocampus-specific gene, which is the gene No.306 on FIG. 49.

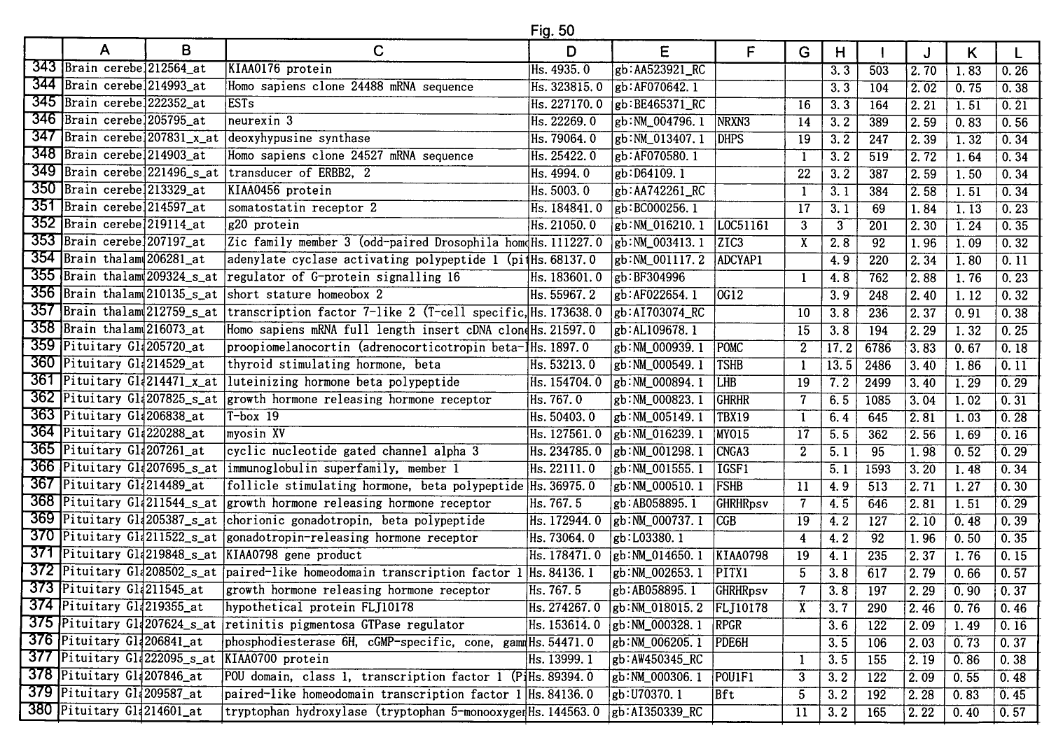

22. A human cerebellum-specific gene selected from the group consisting of genes Nos.307 to 353 on FIGS. 49 to 50, or a set of genes consisting of two or more of human cerebellum-specific genes selected from said group.

23. A human thalamus-specific gene selected from the group consisting of genes Nos.354 to 358 on FIG. 50, or a set of genes consisting of two or more of human thalamus-specific genes selected from said group.

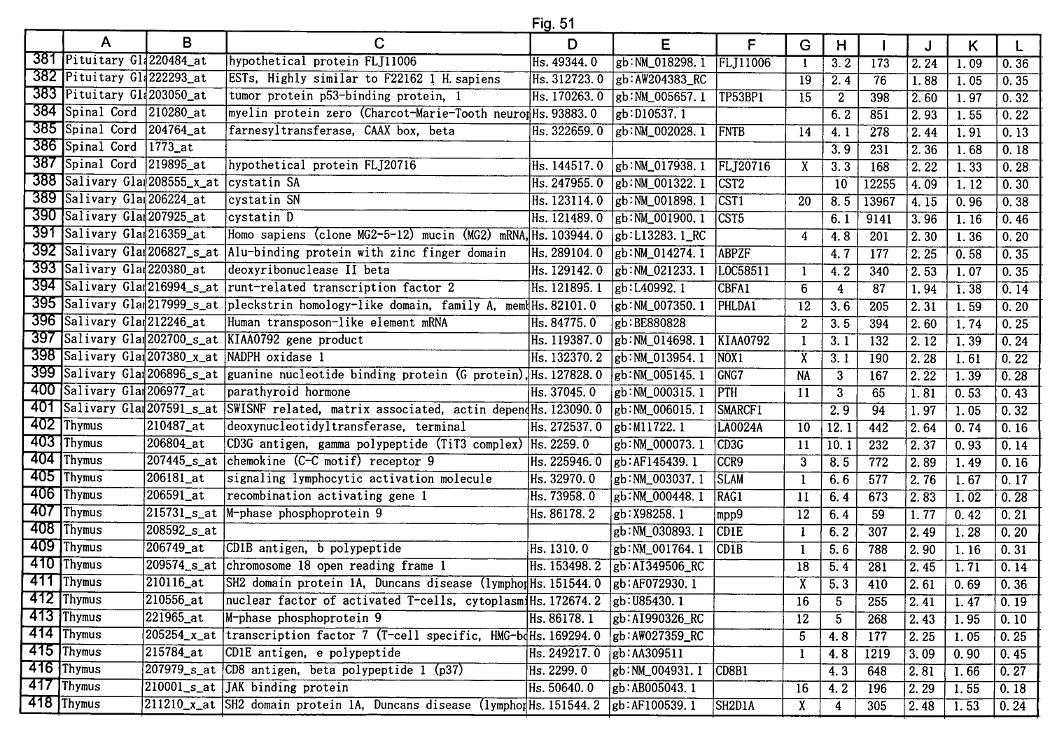

24. A human pituitary gland-specific gene selected from the group consisting of genes Nos.359 to 383 on FIGS. 50 to 51, or a set of genes consisting of two or more of human pituitary gland-specific genes selected from said group.

25. A human spinal cord-specific gene selected from the group consisting of genes Nos.384 to 387 on FIG. 51, or a set of genes consisting of two or more of human spinal cord-specific genes selected from said group.

26. A human salivary gland-specific gene selected from the group consisting of genes Nos.388 to 401 on FIG. 51, or a set of genes consisting of two or more of human salivary gland-specific genes selected from said group.

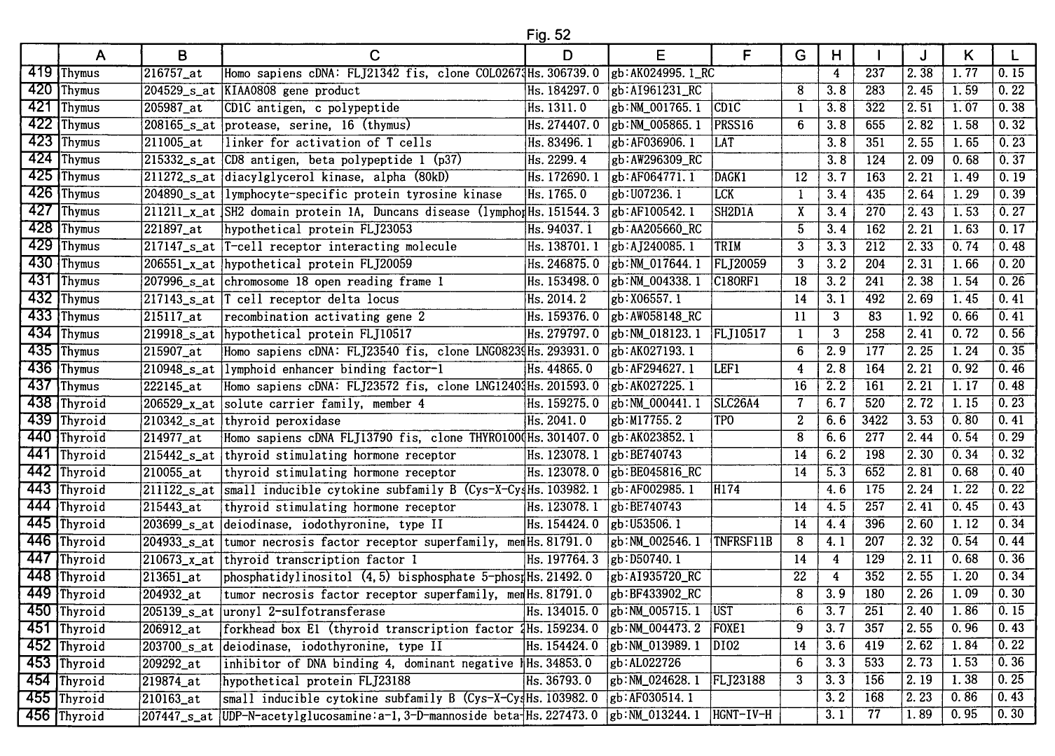

27. A human thymus-specific gene selected from the group consisting of genes Nos.402 to 437 on FIGS. 51 to 52, or a set of genes consisting of two or more of human thymus-specific genes selected from said group.

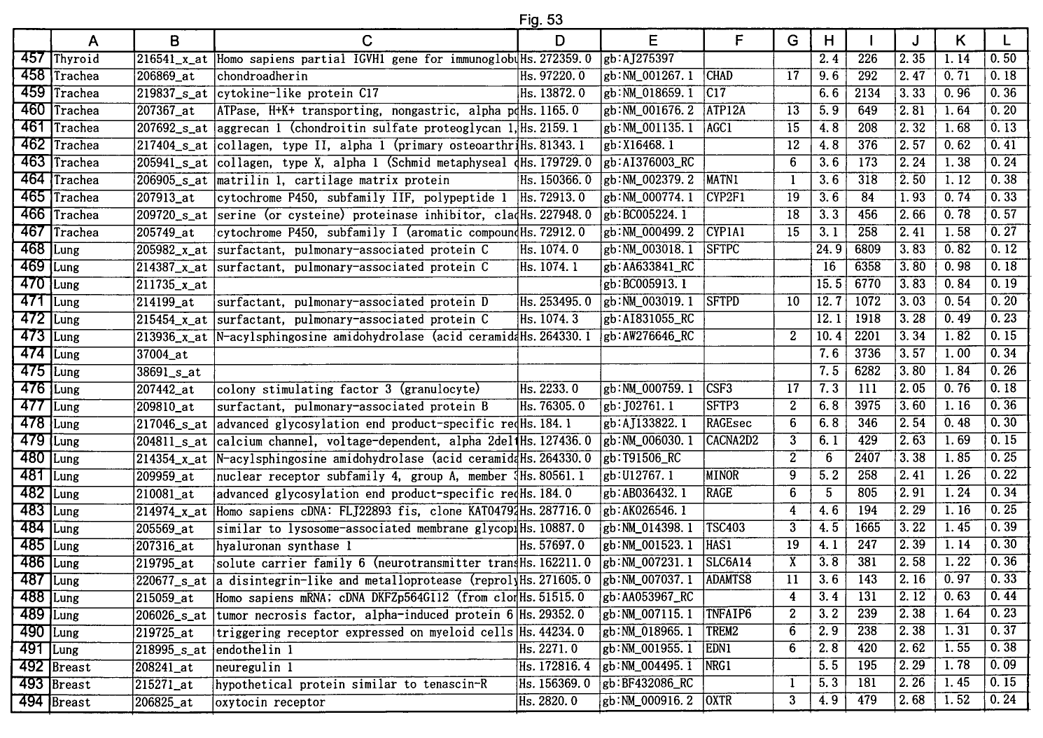

28. A human thyroid gland-specific gene selected from the group consisting of genes Nos.438 to 457 on FIGS. 52 to 53, or a set of genes consisting of two or more of human thyroid gland-specific genes selected from said group.

29. A human trachea-specific gene selected from the group consisting of genes Nos.458 to 467 on FIG. 53, or a set of genes consisting of two or more of human trachea-specific genes selected from said group.

30. A human lung-specific gene selected from the group consisting of genes Nos.468 to 491 on FIG. 53, or a set of genes consisting of two or more of human lung-specific genes selected from said group.

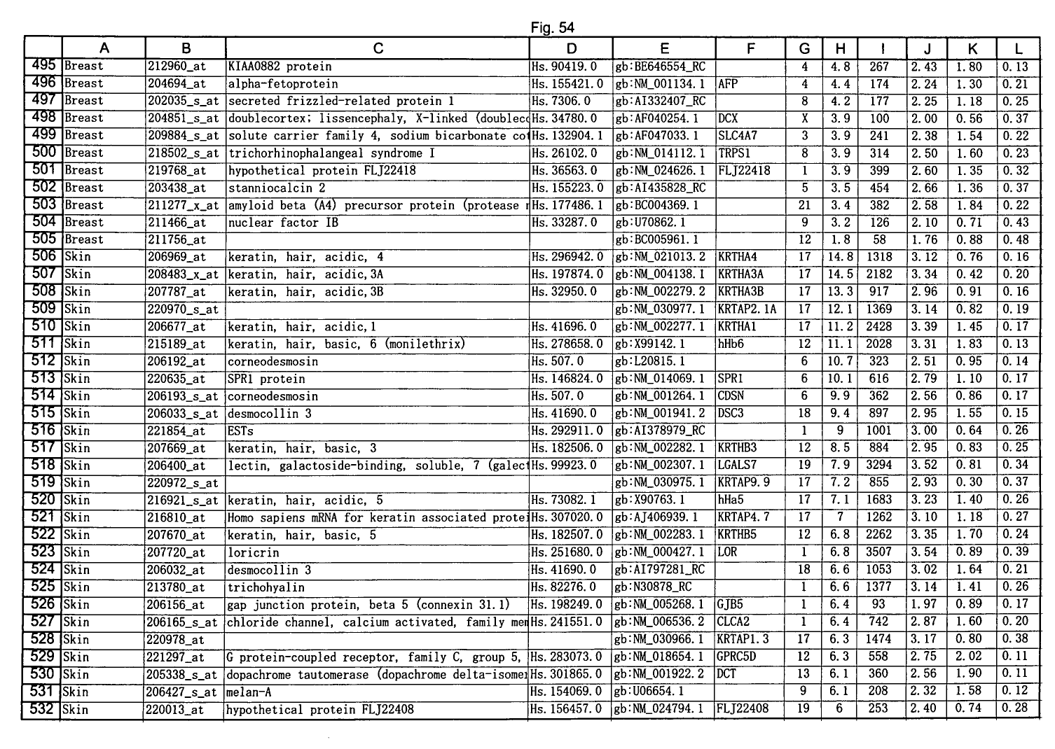

31. A human chest-specific gene selected from the group consisting of genes Nos.492 to 505 on FIGS. 53 to 54, or asset of genes consisting of two or more of human chest-specific genes selected from said group.

32. A human skin-specific gene selected from the group consisting of genes Nos.506 to 577 on FIGS. 54 to 56, or a set of genes consisting of two or more of human skin-specific genes selected from said group.

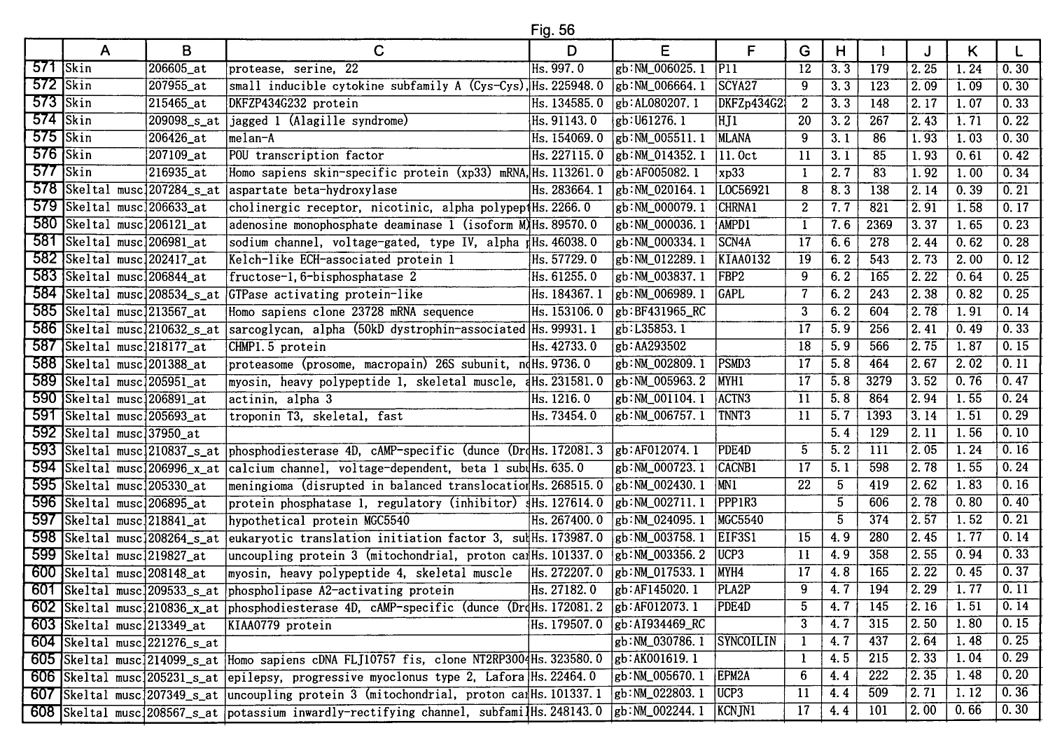

33. A human skeletal muscle-specific gene selected from the group consisting of genes Nos.578 to 650 on FIGS. 56 to 58, or asset of genes consisting of two or more of human skeletal muscle-specific genes selected from said group.

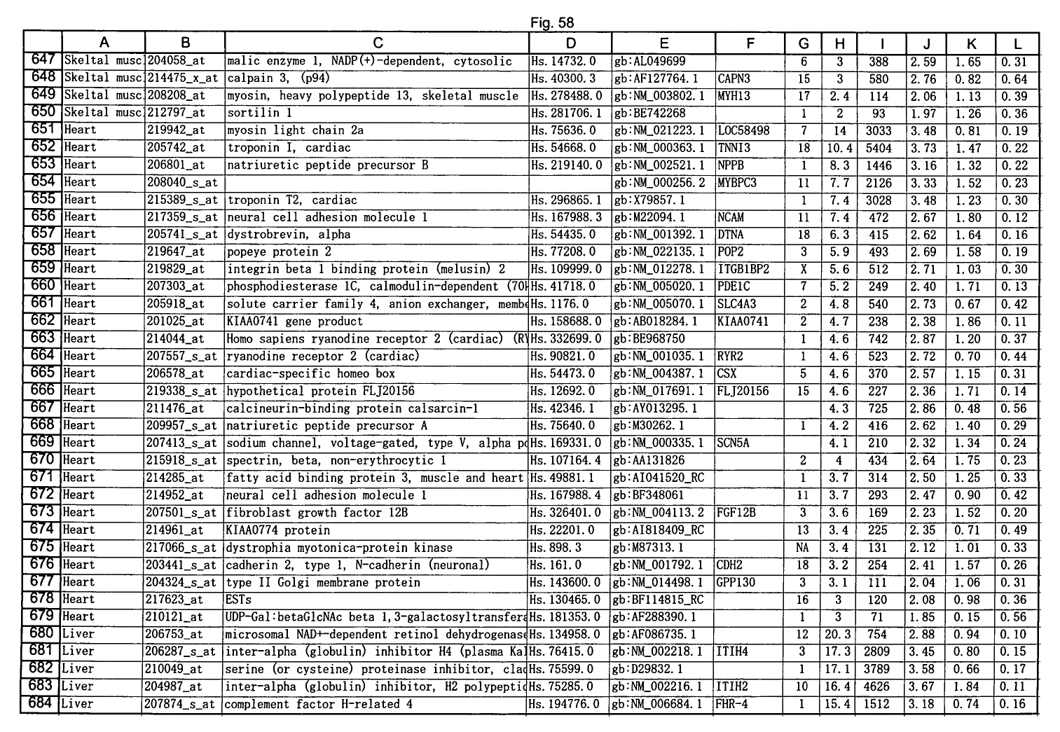

34. A human heart-specific gene selected from the group consisting of genes Nos.651 to 679 on FIG. 58, or a set of genes consisting of two or more of human heart-specific genes selected from said group.

35. A human liver-specific gene selected from the group consisting of genes Nos.680 to 852 on FIGS. 58 to 63, or a set of genes consisting of two or more of human liver-specific genes selected from said group.

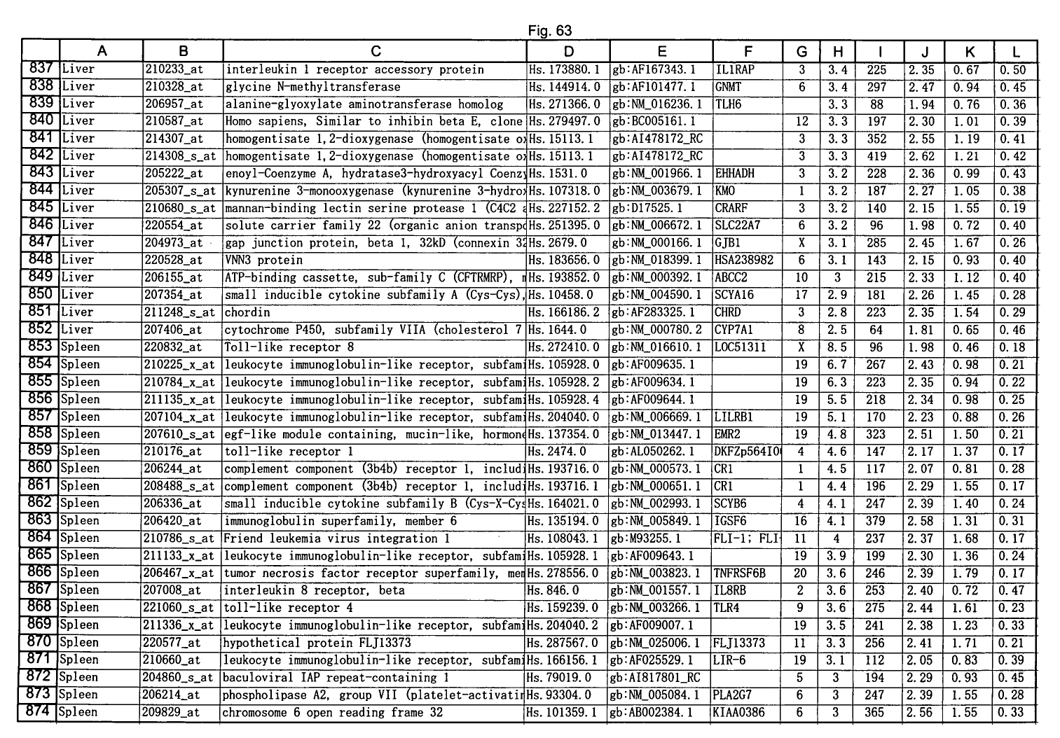

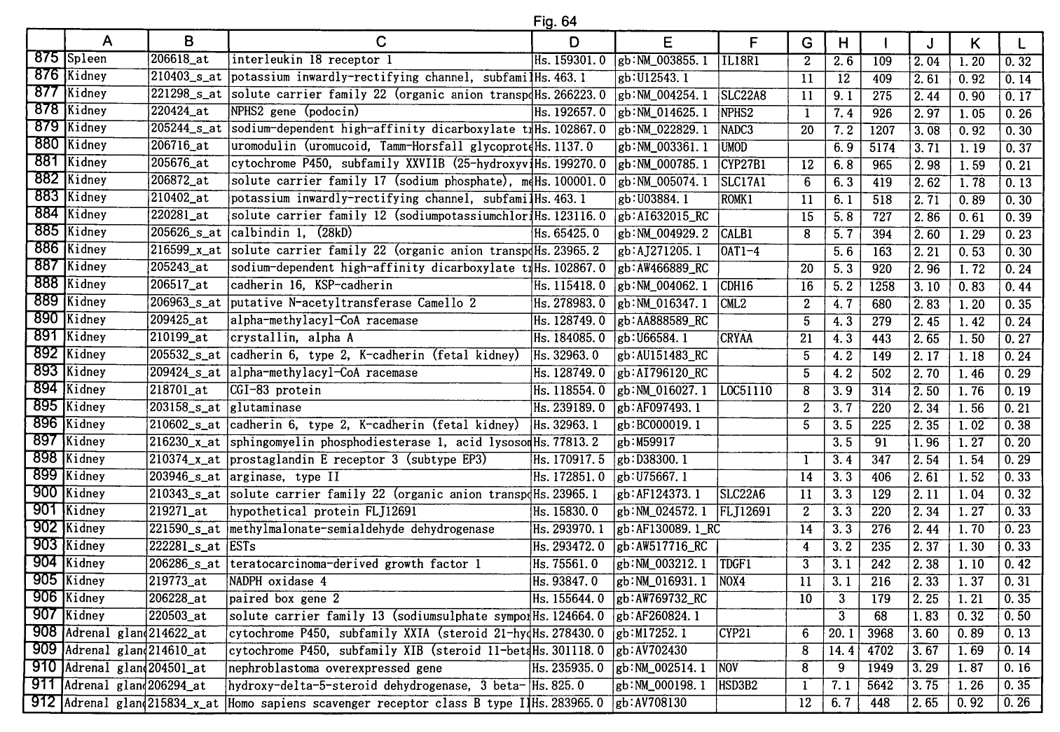

36. A human spleen-specific gene selected from the group consisting of genes Nos.853 to 875 on FIGS. 63 to 64, or a set of genes consisting of two or more of human spleen-specific genes selected from said group.

37. A human kidney-specific gene selected from the group consisting of genes Nos.876 to 907 on FIG. 64, or a set of genes consisting of two or more of human kidney-specific genes selected from said group.

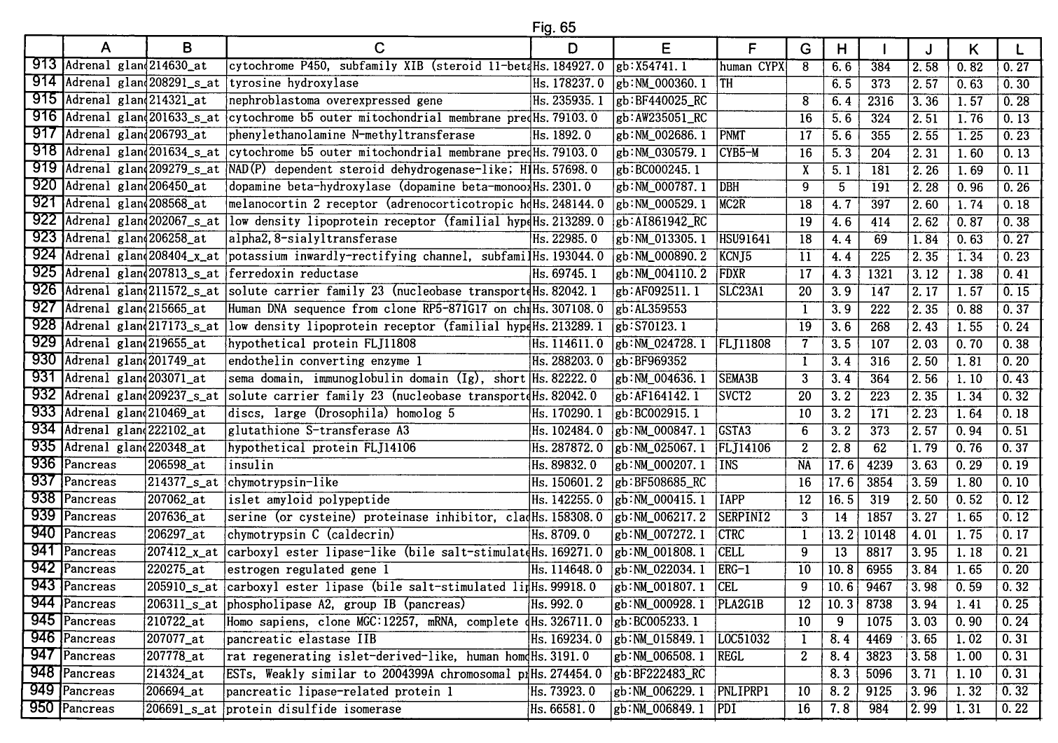

38. A human adrenal gland-specific gene selected from the group consisting of genes Nos.908 to 935 on FIGS. 64 to 65, or a set of genes consisting of two or more of human adrenal gland-specific genes selected from said group.

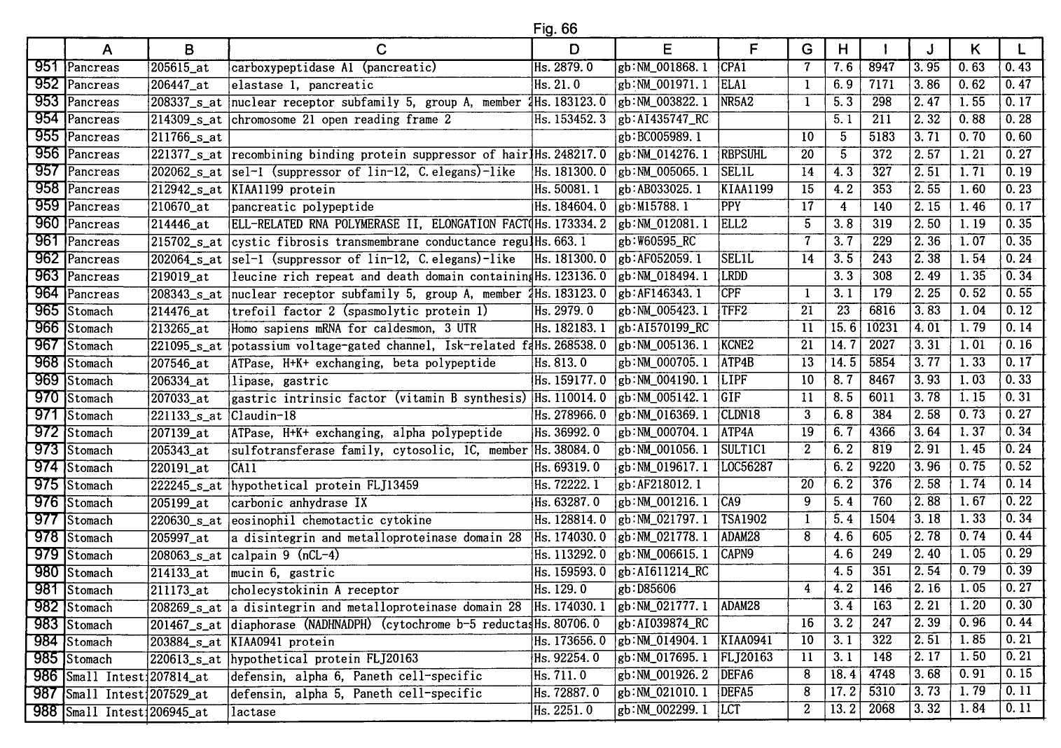

39. A human pancreas-specific gene selected from the group consisting of genes Nos.936 to 964 on FIGS. 65 to 66, or a set of genes consisting of two or more of human pancreas-specific genes selected from said group.

40. A human stomach-specific gene selected from the group consisting of genes Nos.965 to 985 on FIG. 66, or a set of genes consisting of two or more of human stomach-specific genes selected from said group.

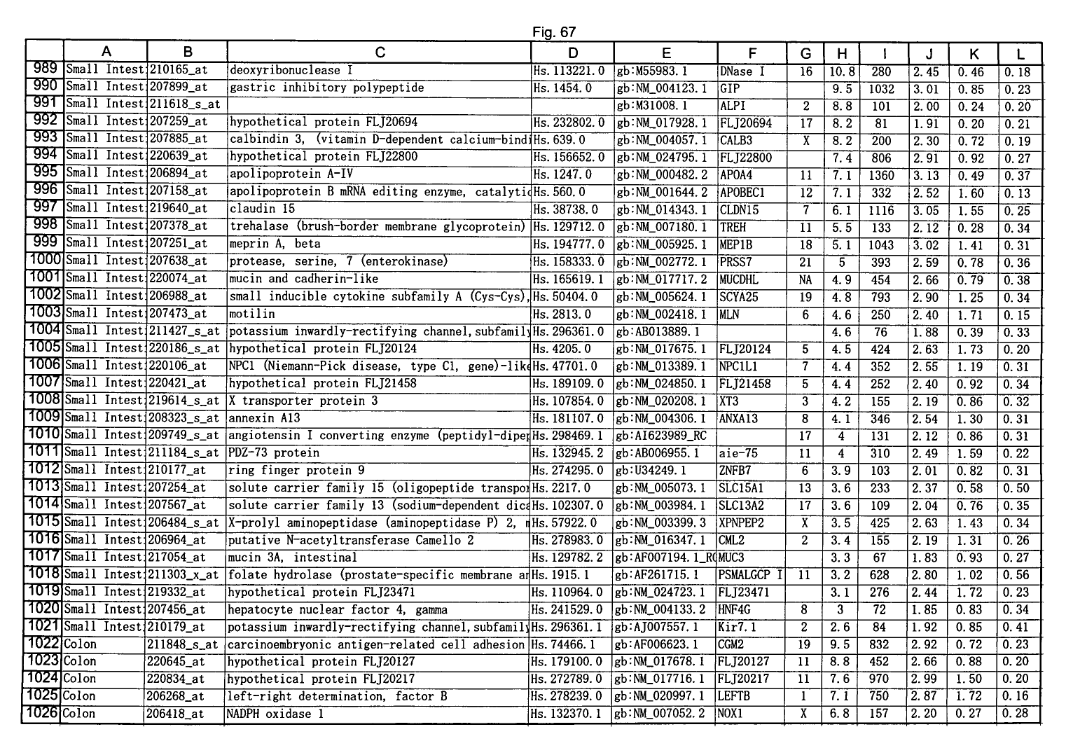

41. A human small intestine-specific gene selected from the group consisting of genes Nos.986 to 1021 on FIGS. 66 to 67, or a set of genes consisting of two or more of human small intestine-specific genes selected from said group.

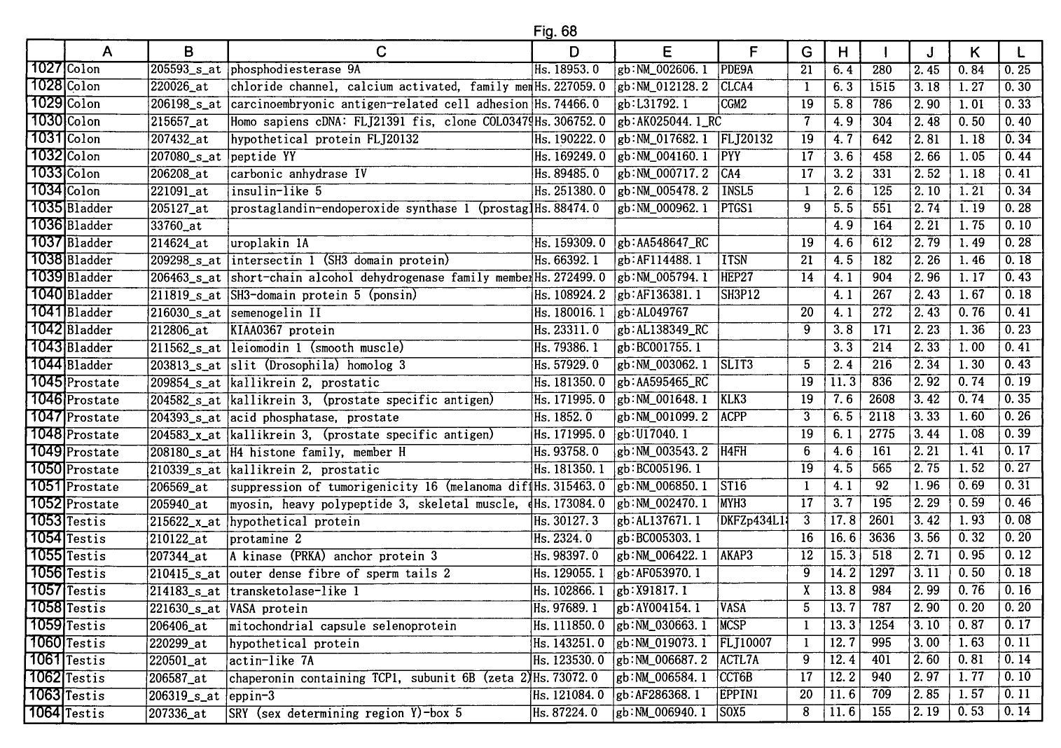

42. A human large intestine-specific gene selected from the group consisting of genes Nos.1022 to 1034 on FIGS. 67 to 68, or a set of genes consisting of two or more of human large intestine-specific genes selected from said group.

43. A human urinary bladder-specific gene selected from the group consisting of genes Nos.1035 to 1044 on FIG. 68, or a set of genes consisting of two or more of human urinary bladder-specific genes selected from said group.

44. A human prostate-specific gene selected from the group consisting of genes Nos.1045 to 1052 on FIG. 68, or a set of genes consisting of two or more of human prostate-specific genes selected from said group.

45. A human testis-specific gene selected from the group consisting of genes Nos.1053 to 1459 on FIGS. 68 to 79, or a set of genes consisting of two or more of human testis-specific genes selected from said group.

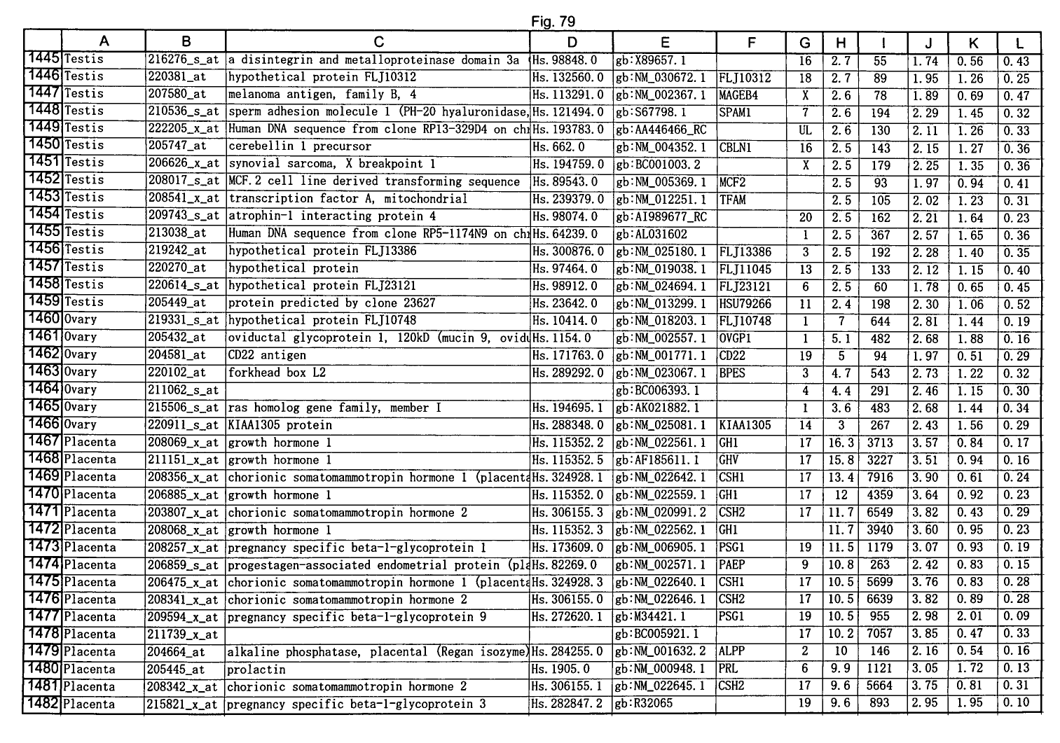

46. A human ovary-specific gene selected from the group consisting of genes Nos.1460 to 1466 on FIG. 79, or a set of genes consisting of two or more of human ovary-specific genes selected from said group.

47. A human placenta-specific gene selected from the group consisting of genes Nos.1467 to 1561 on FIGS. 79 to 82, or a set of genes consisting of two or more of human placenta-specific genes selected from said group.

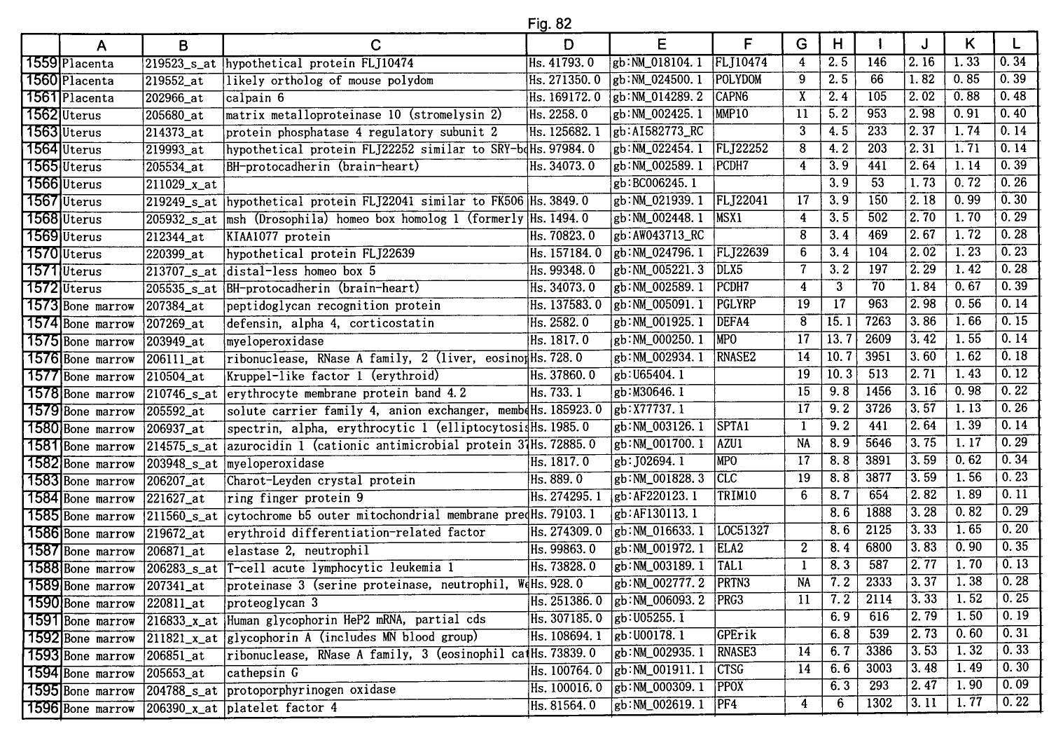

48. A human uterus-specific gene selected from the group consisting of genes Nos.1562 to 1572 on FIG. 82, or a set of genes consisting of two or more of human uterus-specific genes selected from said group.

49. A human bone marrow-specific gene selected from the group consisting of genes Nos.1573 to 1647 on FIGS. 82 to 84, or a set of genes consisting of two or more of human bone marrow-specific genes selected from said group.

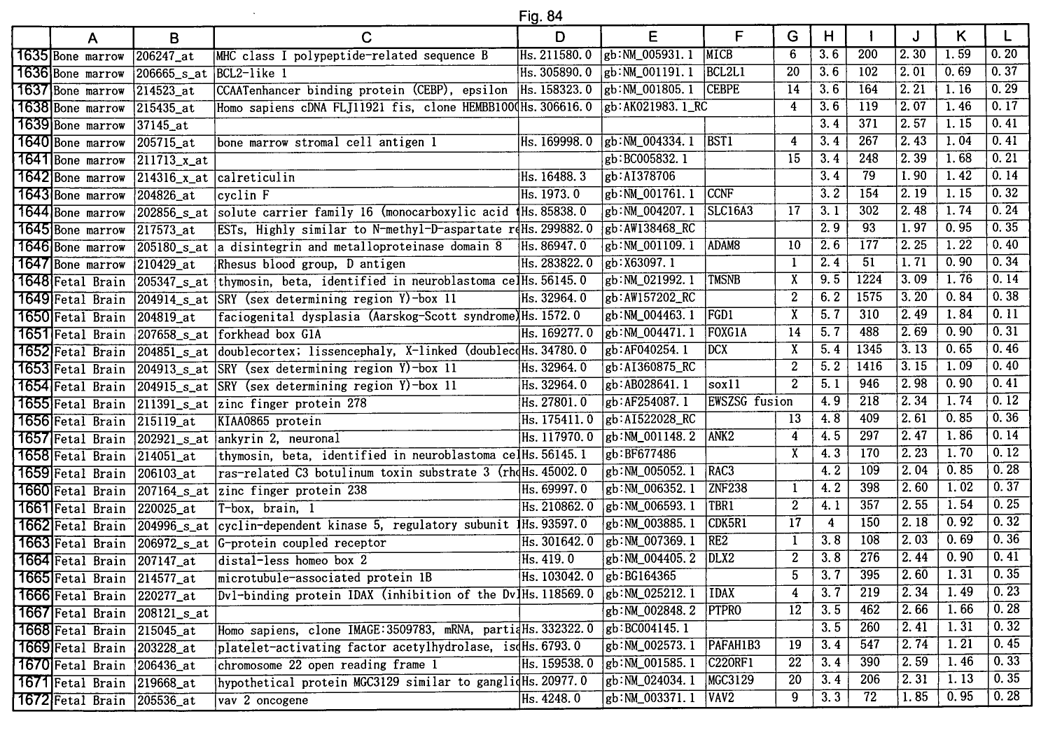

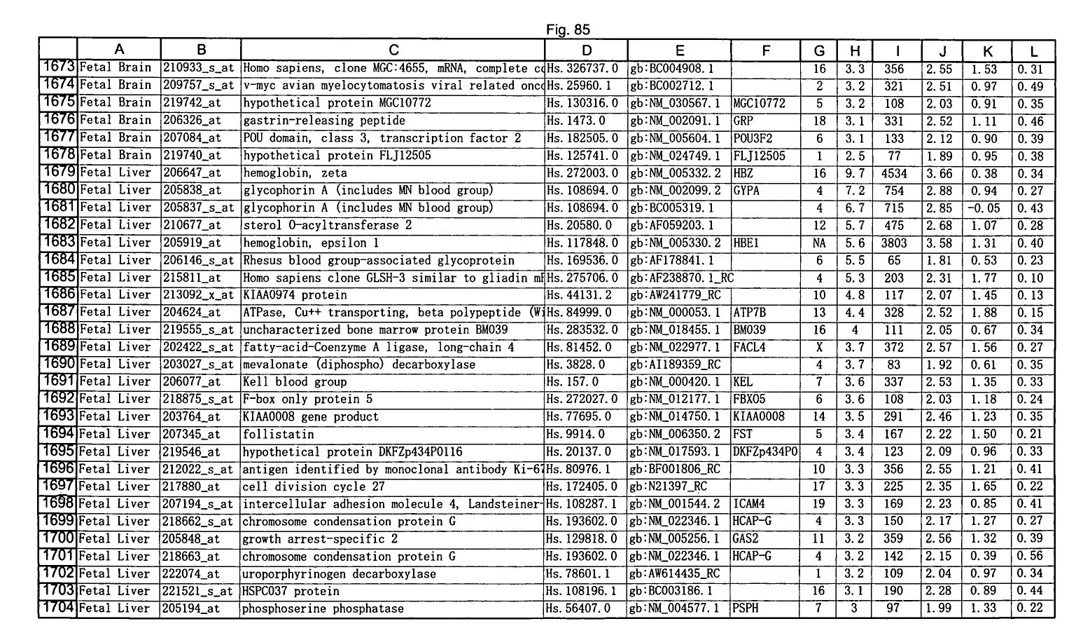

50. A human fetal brain-specific gene selected from the group consisting of genes Nos.1648 to 1678 on FIGS. 84 to 85, or a set of genes consisting of two or more of human fetal brain-specific genes selected from said group.

51. A human fetal liver-specific gene selected from the group consisting of genes Nos.1679 to 1704 on FIG. 85, or a set of genes consisting of two or more of human fetal liver-specific genes selected from said group.

52. A transcription product of any of the genes of claim 17.

53. The transcription product of claim 52, which is an RNA or cDNA.

54. An oligonucleotide probe, which hybridizes with any of the gene of claim 17.

55. A set of transcription products from each gene of any set of genes of claim 17.

56. The set of transcription products of claim 55, which is a set of an RNA or cDNA.

57. A set of oligonucleotide probes, wherein each probe hybridizes with each gene of any set of genes of claim 17.

58. A DNA microarray carrying the set of transcription products of claim 56.
